(19) United States
(12) Patent Application Publication
(10) Pub. No.: US 2024/0261302 A1
(43) Pub. Date: Aug. 8, 2024

(54) METHODS OF TREATING NEURODEGENERATIVE DISEASES OR CONDITIONS

(71) Applicants: FloraScience Inc., Milwaukie, OR (US); The Salk Institute for Biological Studies, San Diego, CA (US)

(72) Inventors: Robert Paul Jensen, Portland, OR (US); Pamela Maher, San Diego, CA (US); Zhibin Liang, San Diego, CA (US)

(21) Appl. No.: 18/403,664

(22) Filed: Jan. 3, 2024

Related U.S. Application Data
(60) Provisional application No. 63/437,100, filed on Jan. 4, 2023.

(51) Int. Cl. A61K 31/00; A61P 25/28

(52) U.S. Cl. CPC A61K 31/658; A61P 25/28

(57) ABSTRACT

Provided are methods of treating a neurodegenerative disease or condition in a subject in need thereof including administering a cannabinoid to the subject.

### CLAIM OF PRIORITY

This application claims priority to U.S. Provisional Application No. 63/437,100 filed on Jan. 4, 2023; the entire contents of which are hereby incorporated by reference.

### STATEMENT REGARDING FEDERALLY SPONSORED RESEARCH

This invention was made with government support under Contract Nos. R01AG069206, RF1AG054714 and R21AG064287 awarded by the National Institutes of Health. The government has certain rights in the invention.

### TECHNICAL FIELD

This disclosure relates to methods of treating a neurodegenerative disease or condition in a subject that include administering a cannabinoid to the subject.

### BACKGROUND

A challenge in modern medicine is to enhance healthy aging and prevent or cure the diseases associated with aging. Aging leads to progressive and detrimental changes in the brain, and old age is the greatest risk factor and likely a driving force for most neurodegenerative diseases including Alzheimer's disease (AD), Parkinson's disease (PD), and Huntington's disease (HD).

Energy production from glucose metabolism supports the majority of brain activity but declines with age and to a greater extent in neurodegenerative diseases. Most of the cerebral energy derived from glucose oxidation is produced in mitochondria. It is believed that mitochondrial inefficiency and/or dysfunction in the brain with aging appears to be one of the pathophysiological commonalities contributing to neurodegeneration. Oxytosis/ferroptosis is a novel non-apoptotic, regulated cell death pathway that recapitulates many features of mitochondrial dysfunction associated with neuronal cell death and has been implicated in age-associated neurodegenerative diseases. However, the fundamental mechanisms by which mitochondrial signaling and phenotypes are altered in the context of oxytosis/ferroptosis and whether pharmacological maintenance of mitochondrial homeostasis can protect against oxytosis/ferroptosis are not fully understood.

### SUMMARY

Provided in the present disclosure are methods of treating a neurodegenerative disease or condition in a subject, the method including administering a therapeutically effective amount of a cannabinoid to the subject. In some embodiments, the cannabinoid is an oxytosis/ferroptosis inhibitor. In some embodiments, the cannabinoid is a neuroprotector. In some embodiments, the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

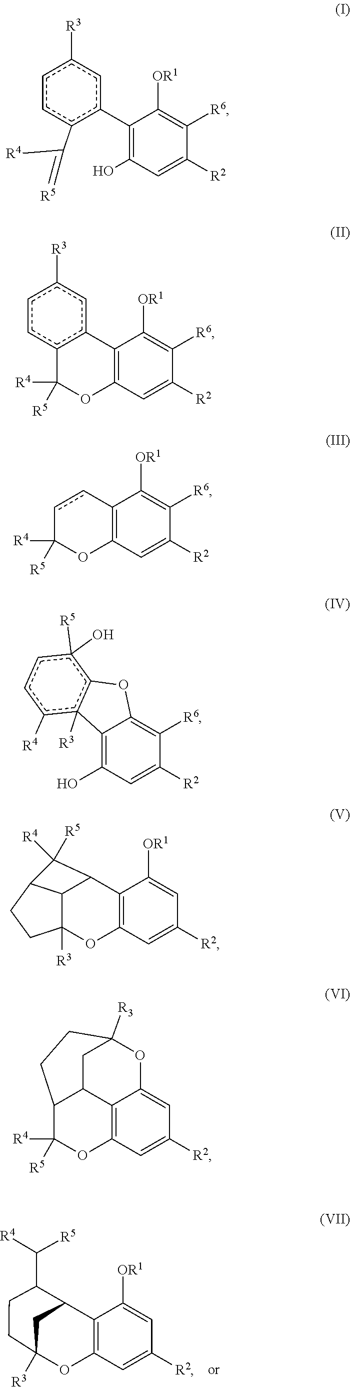

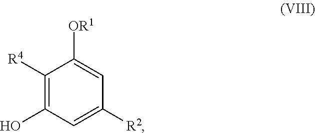

or a pharmaceutically acceptable salt thereof, wherein: each [symbol] is independently a single bond or a double bond; R¹ is hydrogen, C₁-C₁₀ alkyl, C(═O)—(C₁-C₁₀ alkyl), or Si(C₁-C₁₀ alkyl)₃; R² is C₁-C₁₀ alkyl; R³ is C₁-C₆ alkyl optionally substituted with OH; R⁴ is C₁-C₁₀ alkyl or C₂-C₁₀ alkenyl; R⁵ is absent, C₁-C₁₀ alkyl or C₂-C₁₀ alkenyl; and R⁶ is hydrogen, C₁-C₆ alkyl, C(═O)(OH), or C(═O)—(C₁-C₆ alkyl).

In some embodiments, the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN-OMc), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof. In some embodiments, the cannabinoid is selected from the group consisting of cannabinol, acetylcannabinol, dihydrocannabinodiol, cannabichromene, cannabigerol, cannabidiol, and combinations thereof. In some embodiments, the cannabinoid is selected from the group consisting of cannabinol, acetylcannabinol, dihydrocannabinodiol, and combinations thereof. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

In some embodiments, the cannabinoid has a purity of at least about 95%, at least about 96%, at least about 97%, at least about 98%, at least about 99%, at least about 99.5%, or at least about 99.9%. In some embodiments, the cannabinoid is cannabinol and has a purity of at least about 95%, at least about 96%, at least about 97%, at least about 98%, at least about 99%, at least about 99.5%, or at least about 99.9%. In some embodiments, the cannabinoid contains about 0.5 wt % THC or less. In some embodiments, the cannabinoid contains about 0.3 wt % THC or less. In some embodiments, the cannabinoid is substantially free of THC. In some embodiments, the THC is Δ⁹⁽¹⁰⁾-THC. In some embodiments, the cannabinoid is substantially free of Δ⁹⁽¹⁰⁾-THC. In some embodiments, the cannabinoid is cannabinol and contains about 0.5 wt % THC or less. In some embodiments, the cannabinoid is cannabinol and contains about 0.3 wt % THC or less. In some embodiments, the cannabinoid is cannabinol and is substantially free of THC. In some embodiments, the cannabinoid is cannabinol and is substantially free of A⁹⁽¹⁰⁾-THC.

In some embodiments, the cannabinoid is cannabifuran and contains about 0.5 wt % THC or less. In some embodiments, the cannabinoid is cannabifuran and contains about 0.3 wt % THC or less. In some embodiments, the cannabinoid is cannabifuran and is substantially free of THC. In some embodiments, the cannabinoid is cannabifuran and is substantially free of Δ⁹⁽¹⁰⁾-THC.

In some embodiments, the cannabinoid is tetrahydrocannabidiol and contains about 0.5 wt % THC or less. In some embodiments, the cannabinoid is tetrahydrocannabidiol and contains about 0.3 wt % THC or less. In some embodiments, the cannabinoid is tetrahydrocannabidiol and is substantially free of THC. In some embodiments, the cannabinoid is tetrahydrocannabidiol and is substantially free of Δ⁹⁽¹⁰⁾-THC.

In some embodiments, the cannabinoid is tetrahydrocannabigerol and contains about 0.5 wt % THC or less. In some embodiments, the cannabinoid is tetrahydrocannabigerol and contains about 0.3 wt % THC or less. In some embodiments, the cannabinoid is tetrahydrocannabigerol and is substantially free of THC. In some embodiments, the cannabinoid is tetrahydrocannabigerol and is substantially free of Δ⁹⁽¹⁰⁾-THC.

In some embodiments, the subject exhibits one or more risk factors or symptoms associated with a neurodegenerative disease or condition or the development of a neurodegenerative disease or condition. In some embodiments, the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the neurodegenerative disease or condition is selected from the group consisting of Parkinson's disease, Alzheimer's disease, prion disease, a motor neuron disease (MND), amyotrophic lateral sclerosis (ALS), and Huntington's disease (HD). In some embodiments, the neurodegenerative disease or condition is an age-associated neurodegenerative disease or condition. In some embodiments, the subject is a mammal. In some embodiments, the subject is a human. In some embodiments, the cannabinoid inhibits oxytosis/ferroptosis independently of cannabinoid receptors.

In some embodiments, treating the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics. In some embodiments, the cannabinoid is administered in an amount of about 1 mg/kg/day to about 50 mg/kg/day, or about 5 mg/kg/day to about 30 mg/kg/day, or about 10 mg/kg/day to 20 mg/kg/day.

Another aspect of the disclosure includes methods of treating a disease or disorder associated with mitochondrial dysfunction associated with an aging brain in a subject, the method including administering a therapeutically effective amount of a cannabinoid to the subject. In some embodiments, the cannabinoid is an oxytosis/ferroptosis inhibitor. In some embodiments, the cannabinoid is a neuroprotector. In some embodiments, the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

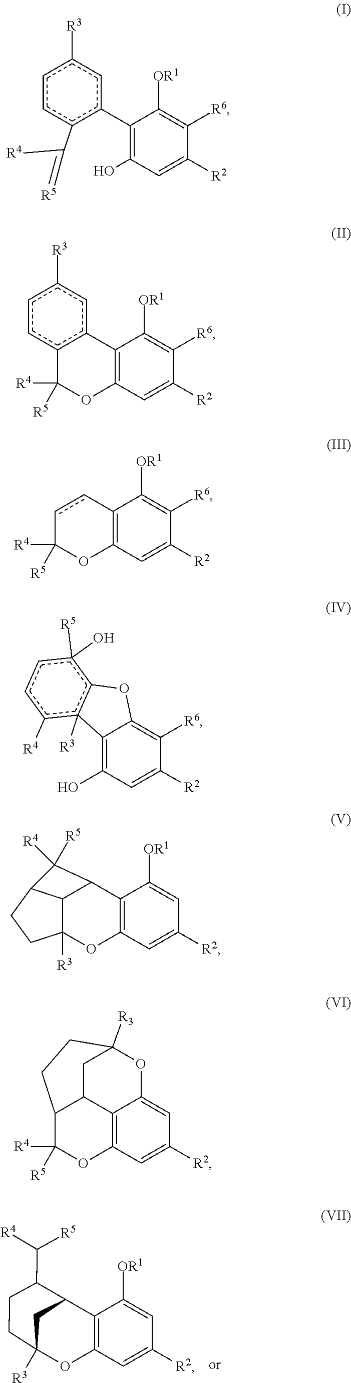

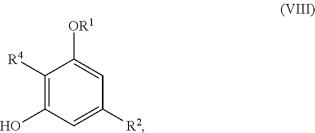

or a pharmaceutically acceptable salt thereof, wherein: each [symbol] is independently a single bond or a double bond; R¹ is hydrogen, C₁-C₁₀ alkyl, C(═O)—(C₁-C₁₀ alkyl), or Si(C₁-C₁₀ alkyl)₃; R² is C₁-C₁₀ alkyl; R³ is C₁-C₆ alkyl optionally substituted with OH; R⁴ is C₁-C₁₀ alkyl or C₂-C₁₀ alkenyl; R⁵ is absent, C₁-C₁₀ alkyl or C₂-C₁₀ alkenyl; and R⁶ is hydrogen, C₁-C₆ alkyl, C(═O)(OH), or C(═O)—(C₁-C₆ alkyl).

In some embodiments, the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof. In some embodiments, the cannabinoid is selected from the group consisting of cannabinol, acetylcannabinol, dihydrocannabinodiol, cannabichromene, cannabigerol, cannabidiol, and combinations thereof. In some embodiments, the cannabinoid is selected from the group consisting of cannabinol, acetylcannabinol, dihydrocannabinodiol, and combinations thereof. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

In some embodiments, the cannabinoid has a purity of at least about 95%, at least about 96%, at least about 97%, at least about 98%, at least about 99%, at least about 99.5%, or at least about 99.9%. In some embodiments, the cannabinoid is cannabinol and has a purity of at least about 95%, at least about 96%, at least about 97%, at least about 98%, at least about 99%, at least about 99.5%, or at least about 99.9%. In some embodiments, the cannabinoid contains about 0.5 wt % THC or less. In some embodiments, the cannabinoid contains about 0.3 wt % THC or less. In some embodiments, the cannabinoid is substantially free of THC. In some embodiments, the THC is Δ⁹⁽¹⁰⁾-THC. In some embodiments, the cannabinoid is substantially free of Δ⁹⁽¹⁰⁾-THC. In some embodiments, the cannabinoid is cannabinol and contains about 0.5 wt % THC or less. In some embodiments, the cannabinoid is cannabinol and contains about 0.3 wt % THC or less. In some embodiments, the cannabinoid is cannabinol and is substantially free of THC. In some embodiments, the cannabinoid is cannabinol and is substantially free of Δ⁹⁽¹⁰⁾-THC.

In some embodiments, the cannabinoid is cannabifuran and contains about 0.5 wt % THC or less. In some embodiments, the cannabinoid is cannabifuran and contains about 0.3 wt % THC or less. In some embodiments, the cannabinoid is cannabifuran and is substantially free of THC. In some embodiments, the cannabinoid is cannabifuran and is substantially free of Δ⁹⁽¹⁰⁾-THC.

In some embodiments, the cannabinoid is tetrahydrocannabidiol and contains about 0.5 wt % THC or less. In some embodiments, the cannabinoid is tetrahydrocannabidiol and contains about 0.3 wt % THC or less. In some embodiments, the cannabinoid is tetrahydrocannabidiol and is substantially free of THC. In some embodiments, the cannabinoid is tetrahydrocannabidiol and is substantially free of Δ⁹⁽¹⁰⁾-THC.

In some embodiments, the cannabinoid is tetrahydrocannabigerol and contains about 0.5 wt % THC or less. In some embodiments, the cannabinoid is tetrahydrocannabigerol and contains about 0.3 wt % THC or less. In some embodiments, the cannabinoid is tetrahydrocannabigerol and is substantially free of THC. In some embodiments, the cannabinoid is tetrahydrocannabigerol and is substantially free of Δ⁹⁽¹⁰⁾-THC.

In some embodiments, the subject exhibits one or more risk factors or symptoms associated with a disease or disorder associated with mitochondrial dysfunction associated with an aging brain or the development of a disease or disorder associated with mitochondrial dysfunction associated with an aging brain. In some embodiments, the disease or disorder associated with mitochondrial dysfunction associated with an aging brain is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the disease or disorder associated with mitochondrial dysfunction associated with an aging brain is selected from the group consisting of Parkinson's disease, Alzheimer's disease, prion disease, a motor neuron disease (MND), amyotrophic lateral sclerosis (ALS), and Huntington's disease (HD).

In some embodiments, the subject is a mammal. In some embodiments, the subject is a human.

In some embodiments, the cannabinoid inhibits oxytosis/ferroptosis independently of cannabinoid receptors. In some embodiments, treating the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics.

In some embodiments, the cannabinoid is administered in an amount of about 1 mg/kg/day to about 50 mg/kg/day, or about 5 mg/kg/day to about 30 mg/kg/day, or about 10 mg/kg/day to 20 mg/kg/day.

Another aspect of the disclosure includes methods of inhibiting oxytosis/ferroptosis in a subject, the method including administering a cannabinoid to the subject. In some embodiments, the cannabinoid is a neuroprotector. In some embodiments, the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

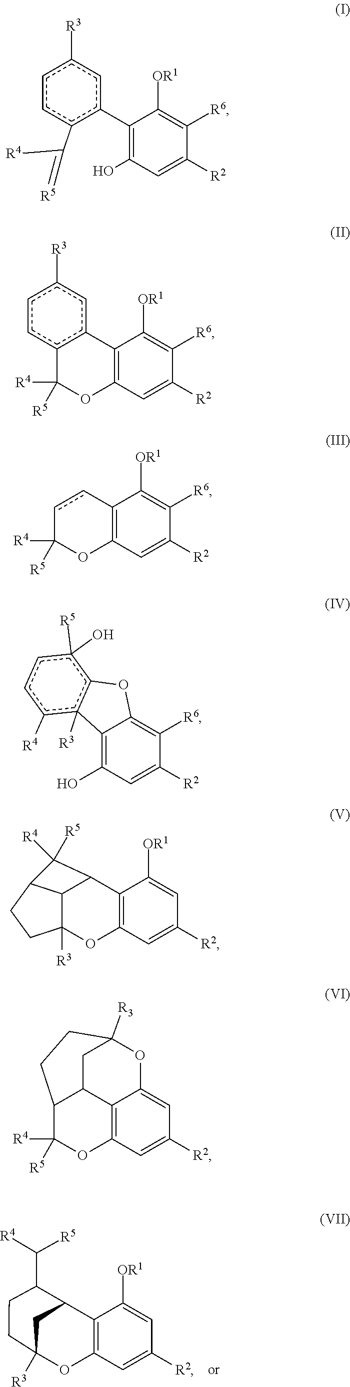

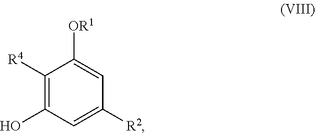

or a pharmaceutically acceptable salt thereof, wherein: each [symbol] is independently a single bond or a double bond; R¹ is hydrogen, C₁-C₁₀ alkyl, C(═O)—(C₁-C₁₀ alkyl), or Si(C₁-C₁₀alkyl)₃; R² is C₁-C₁₀ alkyl; R³ is C₁-C₆ alkyl optionally substituted with OH; R⁴ is C₁-C₁₀ alkyl or C₂-C₁₀ alkenyl; R⁵ is absent, C₁-C₁₀ alkyl or C₂-C₁₀ alkenyl; and R⁶ is hydrogen, C₁-C₆ alkyl, C(═O)(OH), or C(═O)—(C₁-C₆ alkyl). In some embodiments, the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof. In some embodiments, the cannabinoid is selected from the group consisting of cannabinol, acetylcannabinol, dihydrocannabinodiol, cannabichromene, cannabigerol, cannabidiol, and combinations thereof. In some embodiments, the cannabinoid is selected from the group consisting of cannabinol, acetylcannabinol, dihydrocannabinodiol, and combinations thereof. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

In some embodiments, the cannabinoid has a purity of at least about 95%, at least about 96%, at least about 97%, at least about 98%, at least about 99%, at least about 99.5%, or at least about 99.9%. In some embodiments, the cannabinoid is cannabinol and has a purity of at least about 95%, at least about 96%, at least about 97%, at least about 98%, at least about 99%, at least about 99.5%, or at least about 99.9%.

In some embodiments, the cannabinoid contains about 0.5 wt % THC or less. In some embodiments, the cannabinoid contains about 0.3 wt % THC or less. In some embodiments, the cannabinoid is substantially free of THC. In some embodiments, the THC is Δ⁹⁽¹⁰⁾-THC. In some embodiments, the cannabinoid is substantially free of Δ⁹⁽¹⁰⁾-THC. In some embodiments, the cannabinoid is cannabinol and contains about 0.5 wt % THC or less. In some embodiments, the cannabinoid is cannabinol and contains about 0.3 wt % THC or less. In some embodiments, the cannabinoid is cannabinol and is substantially free of THC. In some embodiments, the cannabinoid is cannabinol and is substantially free of Δ⁹⁽¹⁰⁾-THC.

In some embodiments, the cannabinoid is cannabifuran and contains about 0.5 wt % THC or less. In some embodiments, the cannabinoid is cannabifuran and contains about 0.3 wt % THC or less. In some embodiments, the cannabinoid is cannabifuran and is substantially free of THC. In some embodiments, the cannabinoid is cannabifuran and is substantially free of Δ⁹⁽¹⁰⁾-THC.

In some embodiments, the cannabinoid is tetrahydrocannabidiol and contains about 0.5 wt % THC or less. In some embodiments, the cannabinoid is tetrahydrocannabidiol and contains about 0.3 wt % THC or less. In some embodiments, the cannabinoid is tetrahydrocannabidiol and is substantially free of THC. In some embodiments, the cannabinoid is tetrahydrocannabidiol and is substantially free of Δ⁹⁽¹⁰⁾-THC.

In some embodiments, the cannabinoid is tetrahydrocannabigerol and contains about 0.5 wt % THC or less. In some embodiments, the cannabinoid is tetrahydrocannabigerol and contains about 0.3 wt % THC or less. In some embodiments, the cannabinoid is tetrahydrocannabigerol and is substantially free of THC. In some embodiments, the cannabinoid is tetrahydrocannabigerol and is substantially free of Δ⁹⁽¹⁰⁾-THC.

In some embodiments, the subject has a neurodegenerative disease or condition. In some embodiments, the subject exhibits one or more risk factors or symptoms associated with a neurodegenerative disease or condition or the development of a neurodegenerative disease or condition. In some embodiments, the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the neurodegenerative disease or condition is selected from the group consisting of Parkinson's disease, Alzheimer's disease, prion disease, a motor neuron disease (MND), amyotrophic lateral sclerosis (ALS), and Huntington's disease. (HD).

In some embodiments, the subject is a mammal. In some embodiments, the subject is a human.

In some embodiments, the cannabinoid inhibits oxytosis/ferroptosis independently of cannabinoid receptors. In some embodiments, inhibiting oxytosis/ferroptosis in the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal ß-amyloid in neuronal cells, and regulation of mitochondrial dynamics.

In some embodiments, the cannabinoid is administered in an amount of about 1 mg/kg/day to about 50 mg/kg/day, or about 5 mg/kg/day to about 30 mg/kg/day, or about 10 mg/kg/day to 20 mg/kg/day.

Another aspect of the disclosure includes methods of protecting nerve cells from oxytosis/ferroptosis in a subject, the method including administering a cannabinoid to the subject. In some embodiments, the cannabinoid is an oxytosis/ferroptosis inhibitor. In some embodiments, the cannabinoid is a neuroprotector. In some embodiments, the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

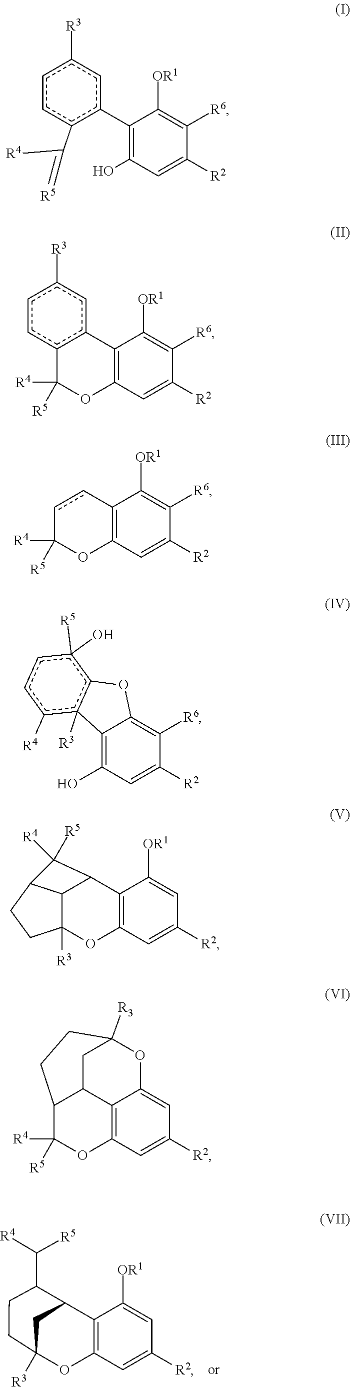

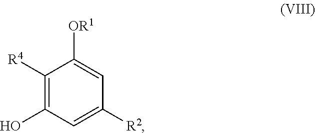

or a pharmaceutically acceptable salt thereof, wherein: each [symbol] is independently a single bond or a double bond; R¹ is hydrogen, C₁-C₁₀ alkyl, C(═O)—(C₁-C₁₀ alkyl), or Si(C₁-C₁₀ alkyl)₃; R² is C₁-C₁₀ alkyl; R³ is C₁-C₆ alkyl optionally substituted with OH; R⁴ is C₁-C₁₀ alkyl or C₂-C₁₀ alkenyl; R⁵ is absent, C₁-C₁₀ alkyl or C₂-C₁₀ alkenyl; and R⁶ is hydrogen, C₁-C₆ alkyl, C(═O)(OH), or C(═O)—(C₁-C₆ alkyl).

In some embodiments, the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof. In some embodiments, the cannabinoid is selected from the group consisting of cannabinol, acetylcannabinol, dihydrocannabinodiol, cannabichromene, cannabigerol, cannabidiol, and combinations thereof. In some embodiments, the cannabinoid is selected from the group consisting of cannabinol, acetylcannabinol, dihydrocannabinodiol, and combinations thereof. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

In some embodiments, the cannabinoid has a purity of at least about 95%, at least about 96%, at least about 97%, at least about 98%, at least about 99%, at least about 99.5%, or at least about 99.9%. In some embodiments, the cannabinoid is cannabinol and has a purity of at least about 95%, at least about 96%, at least about 97%, at least about 98%, at least about 99%, at least about 99.5%, or at least about 99.9%.

In some embodiments, the cannabinoid contains about 0.5 wt % THC or less. In some embodiments, the cannabinoid contains about 0.3 wt % THC or less. In some embodiments, the cannabinoid is substantially free of THC. In some embodiments, the THC is Δ⁹⁽¹⁰⁾-THC. In some embodiments, the cannabinoid is substantially free of Δ⁹⁽¹⁰⁾-THC. In some embodiments, the cannabinoid is cannabinol and contains about 0.5 wt % THC or less. In some embodiments, the cannabinoid is cannabinol and contains about 0.3 wt % THC or less. In some embodiments, the cannabinoid is cannabinol and is substantially free of THC. In some embodiments, the cannabinoid is cannabinol and is substantially free of Δ⁹⁽¹⁰⁾-THC.

In some embodiments, the cannabinoid is cannabifuran and contains about 0.5 wt % THC or less. In some embodiments, the cannabinoid is cannabifuran and contains about 0.3 wt % THC or less. In some embodiments, the cannabinoid is cannabifuran and is substantially free of THC. In some embodiments, the cannabinoid is cannabifuran and is substantially free of Δ⁹⁽¹⁰⁾-THC.

In some embodiments, the cannabinoid is tetrahydrocannabidiol and contains about 0.5 wt % THC or less. In some embodiments, the cannabinoid is tetrahydrocannabidiol and contains about 0.3 wt % THC or less. In some embodiments, the cannabinoid is tetrahydrocannabidiol and is substantially free of THC. In some embodiments, the cannabinoid is tetrahydrocannabidiol and is substantially free of Δ⁹⁽¹⁰⁾-THC.

In some embodiments, the cannabinoid is tetrahydrocannabigerol and contains about 0.5 wt % THC or less. In some embodiments, the cannabinoid is tetrahydrocannabigerol and contains about 0.3 wt % THC or less. In some embodiments, the cannabinoid is tetrahydrocannabigerol and is substantially free of THC. In some embodiments, the cannabinoid is tetrahydrocannabigerol and is substantially free of Δ⁹⁽¹⁰⁾-THC.

In some embodiments, administration of the cannabinoid preserves mitochondrial function. In some embodiments, the subject has a neurodegenerative disease or condition. In some embodiments, the subject exhibits one or more risk factors or symptoms associated with a neurodegenerative disease or condition or the development of a neurodegenerative disease or condition. In some embodiments, the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

In some embodiments, the subject is a mammal. In some embodiments, the subject is a human.

In some embodiments, the cannabinoid inhibits oxytosis/ferroptosis independently of cannabinoid receptors. In some embodiments, inhibiting oxytosis/ferroptosis in the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal ß-amyloid in neuronal cells, and regulation of mitochondrial dynamics.

In some embodiments, the cannabinoid is administered in an amount of about 1 mg/kg/day to about 50 mg/kg/day, or about 5 mg/kg/day to about 30 mg/kg/day, or about 10 mg/kg/day to 20 mg/kg/day.

Another aspect of the disclosure includes methods of treating a neurodegenerative disease or condition in a subject, the method including administering a therapeutically effective amount of cannabinol to the subject, where the cannabinol inhibits oxytosis/ferroptosis. In some embodiments, the cannabinol inhibits oxytosis/ferroptosis independently of cannabinoid receptors.

In some embodiments, the cannabinol has a purity of at least about 95%, at least about 96%, at least about 97%, at least about 98%, at least about 99%, at least about 99.5%, or at least about 99.9%. In some embodiments, the cannabinol contains about 0.5 wt % THC or less. In some embodiments, the cannabinol contains about 0.3 wt % THC or less. In some embodiments, the cannabinol is substantially free of THC. In some embodiments, the THC is Δ⁹⁽¹⁰⁾-THC. In some embodiments, the cannabinol is substantially free of Δ⁹⁽¹⁰⁾-THC.

Another aspect of the disclosure includes methods of treating a neurodegenerative disease or condition in a subject, the method including administering a therapeutically effective amount of cannabifuran to the subject, where the cannabinol inhibits oxytosis/ferroptosis. In some embodiments, the cannabifuran inhibits oxytosis/ferroptosis independently of cannabinoid receptors.

In some embodiments, the cannabifuran has a purity of at least about 95%, at least about 96%, at least about 97%, at least about 98%, at least about 99%, at least about 99.5%, or at least about 99.9%. In some embodiments, the cannabifuran contains about 0.5 wt % THC or less. In some embodiments, the cannabifuran contains about 0.3 wt % THC or less. In some embodiments, the cannabifuran is substantially free of THC. In some embodiments, the THC is Δ⁹⁽¹⁰⁾-THC. In some embodiments, the cannabifuran is substantially free of Δ⁹⁽¹⁰⁾-THC.

Another aspect of the disclosure includes methods of treating a neurodegenerative disease or condition in a subject, the method including administering a therapeutically effective amount of tetrahydrocannabidiol to the subject, where the tetrahydrocannabidiol inhibits oxytosis/ferroptosis. In some embodiments, the tetrahydrocannabidiol inhibits oxytosis/ferroptosis independently of cannabinoid receptors.

In some embodiments, the tetrahydrocannabidiol has a purity of at least about 95%, at least about 96%, at least about 97%, at least about 98%, at least about 99%, at least about 99.5%, or at least about 99.9%. In some embodiments, the tetrahydrocannabidiol contains about 0.5 wt % THC or less. In some embodiments, the tetrahydrocannabidiol contains about 0.3 wt % THC or less. In some embodiments, the tetrahydrocannabidiol is substantially free of THC. In some embodiments, the THC is Δ⁹⁽¹⁰⁾-THC. In some embodiments, the tetrahydrocannabidiol is substantially free of Δ⁹⁽¹⁰⁾-THC.

Another aspect of the disclosure includes methods of treating a neurodegenerative disease or condition in a subject, the method including administering a therapeutically effective amount of tetrahydrocannabigerol to the subject, where the tetrahydrocannabigerol inhibits oxytosis/ferroptosis. In some embodiments, the tetrahydrocannabigerol inhibits oxytosis/ferroptosis independently of cannabinoid receptors.

In some embodiments, the tetrahydrocannabigerol has a purity of at least about 95%, at least about 96%, at least about 97%, at least about 98%, at least about 99%, at least about 99.5%, or at least about 99.9%. In some embodiments, the tetrahydrocannabigerol contains about 0.5 wt % THC or less. In some embodiments, the tetrahydrocannabigerol contains about 0.3 wt % THC or less. In some embodiments, the tetrahydrocannabigerol is substantially free of THC. In some embodiments, the THC is Δ⁹⁽¹⁰⁾-THC. In some embodiments, the tetrahydrocannabigerol is substantially free of Δ⁹⁽¹⁰⁾-THC.

In some embodiments, the subject exhibits one or more risk factors or symptoms associated with a neurodegenerative disease or condition or the development of a neurodegenerative disease or condition. In some embodiments, the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the neurodegenerative disease or condition is selected from the group consisting of Parkinson's disease, Alzheimer's disease, prion disease, a motor neuron disease (MND), amyotrophic lateral sclerosis (ALS), and Huntington's disease (HD). In some embodiments, the neurodegenerative disease or condition is an age-associated neurodegenerative disease or condition.

Another aspect of the disclosure includes methods of treating a disease or disorder associated with mitochondrial dysfunction associated with an aging brain in a subject, the method including administering a therapeutically effective amount of cannabinol to the subject.

In some embodiments, the cannabinol has a purity of at least about 95%, at least about 96%, at least about 97%, at least about 98%, at least about 99%, at least about 99.5%, or at least about 99.9%. In some embodiments, the cannabinol contains about 0.5 wt % THC or less. In some embodiments, the cannabinol contains about 0.3 wt % THC or less. In some embodiments, the cannabinol is substantially free of THC. In some embodiments, the THC is Δ⁹⁽¹⁰⁾-THC. In some embodiments, the cannabinol is substantially free of Δ⁹⁽¹⁰⁾-THC.

Another aspect of the disclosure includes methods of treating a disease or disorder associated with mitochondrial dysfunction associated with an aging brain in a subject, the method including administering a therapeutically effective amount of cannabifuran to the subject.

In some embodiments, the cannabifuran has a purity of at least about 95%, at least about 96%, at least about 97%, at least about 98%, at least about 99%, at least about 99.5%, or at least about 99.9%. In some embodiments, the cannabifuran contains about 0.5 wt % THC or less. In some embodiments, the cannabifuran contains about 0.3 wt % THC or less. In some embodiments, the cannabifuran is substantially free of THC. In some embodiments, the THC is Δ⁹⁽¹⁰⁾-THC. In some embodiments, the cannabifuran is substantially free of Δ⁹⁽¹⁰⁾-THC.

Another aspect of the disclosure includes methods of treating a disease or disorder associated with mitochondrial dysfunction associated with an aging brain in a subject, the method including administering a therapeutically effective amount of tetrahydrocannabidiol to the subject.

In some embodiments, the tetrahydrocannabidiol has a purity of at least about 95%, at least about 96%, at least about 97%, at least about 98%, at least about 99%, at least about 99.5%, or at least about 99.9%. In some embodiments, the tetrahydrocannabidiol contains about 0.5 wt % THC or less. In some embodiments, the tetrahydrocannabidiol contains about 0.3 wt % THC or less. In some embodiments, the tetrahydrocannabidiol is substantially free of THC. In some embodiments, the THC is Δ⁹⁽¹⁰⁾-THC. In some embodiments, the tetrahydrocannabidiol is substantially free of Δ⁹⁽¹⁰⁾-THC.

Another aspect of the disclosure includes methods of treating a disease or disorder associated with mitochondrial dysfunction associated with an aging brain in a subject, the method including administering a therapeutically effective amount of tetrahydrocannabigerol to the subject.

In some embodiments, the tetrahydrocannabigerol has a purity of at least about 95%, at least about 96%, at least about 97%, at least about 98%, at least about 99%, at least about 99.5%, or at least about 99.9%. In some embodiments, the tetrahydrocannabigerol contains about 0.5 wt % THC or less. In some embodiments, the tetrahydrocannabigerol contains about 0.3 wt % THC or less. In some embodiments, the tetrahydrocannabigerol is substantially free of THC. In some embodiments, the THC is Δ⁹⁽¹⁰⁾-THC. In some embodiments, the tetrahydrocannabigerol is substantially free of Δ⁹⁽¹⁰⁾-THC.

In some embodiments, the subject exhibits one or more risk factors or symptoms associated with a disease or disorder associated with mitochondrial dysfunction associated with an aging brain or the development of a disease or disorder associated with mitochondrial dysfunction associated with an aging brain. In some embodiments, the disease or disorder associated with mitochondrial dysfunction associated with an aging brain is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

In some embodiments, the cannabinol is administered in an amount of about 1 mg/kg/day to about 50 mg/kg/day, or about 5 mg/kg/day to about 30 mg/kg/day, or about 10 mg/kg/day to 20 mg/kg/day.

In some embodiments, the cannabifuran is administered in an amount of about 1 mg/kg/day to about 50 mg/kg/day, or about 5 mg/kg/day to about 30 mg/kg/day, or about 10 mg/kg/day to 20 mg/kg/day.

In some embodiments, the tetrahydrocannabidiol is administered in an amount of about 1 mg/kg/day to about 50 mg/kg/day, or about 5 mg/kg/day to about 30 mg/kg/day, or about 10 mg/kg/day to 20 mg/kg/day.

In some embodiments, the tetrahydrocannabigerol is administered in an amount of about 1 mg/kg/day to about 50 mg/kg/day, or about 5 mg/kg/day to about 30 mg/kg/day, or about 10 mg/kg/day to 20 mg/kg/day.

Another aspect of the disclosure includes methods of protecting nerve cells from oxytosis/ferroptosis in a subject, the method including administering cannabinol to the subject.

In some embodiments, the cannabinol has a purity of at least about 95%, at least about 96%, at least about 97%, at least about 98%, at least about 99%, at least about 99.5%, or at least about 99.9%. In some embodiments, the cannabinol contains about 0.5 wt % THC or less. In some embodiments, the cannabinol contains about 0.3 wt % THC or less. In some embodiments, the cannabinol is substantially free of THC. In some embodiments, the THC is Δ⁹⁽¹⁰⁾-THC. In some embodiments, the cannabinol is substantially free of Δ⁹⁽¹⁰⁾-THC.

Another aspect of the disclosure includes methods of protecting nerve cells from oxytosis/ferroptosis in a subject, the method including administering cannabifuran to the subject.

In some embodiments, the cannabifuran has a purity of at least about 95%, at least about 96%, at least about 97%, at least about 98%, at least about 99%, at least about 99.5%, or at least about 99.9%. In some embodiments, the cannabifuran contains about 0.5 wt % THC or less. In some embodiments, the cannabifuran contains about 0.3 wt % THC or less. In some embodiments, the cannabifuran is substantially free of THC. In some embodiments, the THC is Δ⁹⁽¹⁰⁾-THC. In some embodiments, the cannabifuran is substantially free of Δ⁹⁽¹⁰⁾-THC.

Another aspect of the disclosure includes methods of protecting nerve cells from oxytosis/ferroptosis in a subject, the method including administering tetrahydrocannabidiol to the subject.

In some embodiments, the tetrahydrocannabidiol has a purity of at least about 95%, at least about 96%, at least about 97%, at least about 98%, at least about 99%, at least about 99.5%, or at least about 99.9%. In some embodiments, the tetrahydrocannabidiol contains about 0.5 wt % THC or less. In some embodiments, the tetrahydrocannabidiol contains about 0.3 wt % THC or less. In some embodiments, the tetrahydrocannabidiol is substantially free of THC. In some embodiments, the THC is Δ⁹⁽¹⁰⁾-THC. In some embodiments, the tetrahydrocannabidiol is substantially free of Δ⁹⁽¹⁰⁾-THC.

Another aspect of the disclosure includes methods of protecting nerve cells from oxytosis/ferroptosis in a subject, the method including administering tetrahydrocannabigerol to the subject.

In some embodiments, the tetrahydrocannabigerol has a purity of at least about 95%, at least about 96%, at least about 97%, at least about 98%, at least about 99%, at least about 99.5%, or at least about 99.9%. In some embodiments, the tetrahydrocannabigerol contains about 0.5 wt % THC or less. In some embodiments, the tetrahydrocannabigerol contains about 0.3 wt % THC or less. In some embodiments, the tetrahydrocannabigerol is substantially free of THC. In some embodiments, the THC is Δ⁹⁽¹⁰⁾-THC. In some embodiments, the tetrahydrocannabigerol is substantially free of Δ⁹⁽¹⁰⁾-THC.

In some embodiments, the subject exhibits one or more risk factors or symptoms associated with a neurodegenerative disease or condition or the development of a neurodegenerative disease or condition. In some embodiments, the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the neurodegenerative disease or condition is selected from the group consisting of Parkinson's disease, Alzheimer's disease, prion disease, a motor neuron disease (MND), amyotrophic lateral sclerosis (ALS), and Huntington's disease (HD). In some embodiments, the neurodegenerative disease or condition is an age-associated neurodegenerative disease or condition.

In some embodiments, the cannabinol is administered in an amount of about 1 mg/kg/day to about 50 mg/kg/day, or about 5 mg/kg/day to about 30 mg/kg/day, or about 10 mg/kg/day to 20 mg/kg/day.

In some embodiments, the cannabifuran is administered in an amount of about 1 mg/kg/day to about 50 mg/kg/day, or about 5 mg/kg/day to about 30 mg/kg/day, or about 10 mg/kg/day to 20 mg/kg/day.

In some embodiments, the tetrahydrocannabidiol is administered in an amount of about 1 mg/kg/day to about 50 mg/kg/day, or about 5 mg/kg/day to about 30 mg/kg/day, or about 10 mg/kg/day to 20 mg/kg/day.

In some embodiments, the tetrahydrocannabigerol is administered in an amount of about 1 mg/kg/day to about 50 mg/kg/day, or about 5 mg/kg/day to about 30 mg/kg/day, or about 10 mg/kg/day to 20 mg/kg/day.

Another aspect of the disclosure includes dosage forms containing a pharmaceutically acceptable excipient and a therapeutically effective amount of a cannabinoid. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 shows a qPCR analysis of the CB1 and CB2 receptor expression in mouse HT22 nerve cells. Receptor expression levels were normalized to mouse hippocampus (CB1), or mouse cerebellum (CB2).

FIGS. 2A-2I show CBN inhibits oxytosis/ferroptosis in HT22 cells. Representative micrographs of HT22 cells following treatment for 16 h: (FIG. 2A) 0.2% ethanol treatment as vehicle control, (FIG. 2B) 5 μM CBN treatment, (FIG. 2C) 5 mM glutamate treatment, (FIG. 2D) 50 nM RSL3 treatment, (FIG. 2E) 5 μM CBN pretreatment for 1 h followed by 5 mM glutamate treatment, (FIG. 2F) 5 μM CBN pretreatment for 1 h followed by 50 nM RSL3 treatment. Micrographs show the representative morphological characteristics of the cell cultures under a given condition of 16 experimental replicates. Scale bar=100 μm. (FIG. 2G) Cells were pretreated with varying concentrations of CBN for 1 h followed by 5 mM glutamate treatment and incubation for 16 h. (FIG. 2H) Cells were pretreated with varying concentrations of CBN for 1 h followed by 50 nM RSL3 treatment and incubation for 16 h. The results are presented as the percentage of the neuroprotective activity relative to control (100%) and Glu/RSL3 (0%). The neuroprotection curves were analyzed by four-parameter regression. (FIG. 2I) Cytotoxicity assessment of CBN in HT22 cells. Cells were treated with increasing concentrations of CBN or 0.2% ethanol vehicle and incubated for 16 h. Data are the mean of 8-16 replicates per condition±SD.

FIG. 3 shows the cytotoxicity profiles of CBN in different cell lines. The cells were treated with increasing concentrations of CBN for 16 hr.

FIGS. 4A-4I show CBN prevents oxidative stress induced during oxytosis/ferroptosis in HT22 cells. (FIG. 4A) Cellular ROS levels upon different treatment conditions in the cells for 16 h. Data were normalized to total protein/well and are the mean of 16 replicates per condition±SD. (FIG. 4B) Mitochondrial ROS levels upon different treatments in the cells for 16 h. Data were normalized to total protein/well and are the mean of 16 replicates per condition±SD. (FIG. 4C) Cellular lipid peroxidation levels upon different treatment conditions of the cells for 16 h. Data were normalized to total protein/well and are the mean of 12 replicates per condition±SD. (FIG. 4D) Time course of lipid peroxidation levels following different treatment conditions in a cell-free system. Data are the mean of 4 replicates per condition±SD. (FIG. 4E) Trolox equivalent antioxidant capacities (TEAC) following different treatment conditions in a cell-free system. Data are the mean of 8 replicates per condition±SD. The results are presented as the percentage of the TEAC relative to Trolox (100%). (FIG. 4F) Iron (II) binding capacities following different treatment conditions in a cell-free system. Data are the mean of 8 replicates per condition±SD. The results are presented as the percentage relative to the maximum (vehicle without iron, 100%) and minimum (vehicle with iron only, 0%) Fe2+ binding capacity. (FIG. 4G) Total GSH levels upon different treatment conditions of the cells for 16 h. Data are the mean of 4 replicates per condition±SD. (FIG. 4H) Western blot data of Nrf2, ATF4, HO-1, SOD2, GPX4, HSP60, and actin (n=3−6). Protein levels were measured upon different treatment conditions of the cells for 16 h. (FIG. 4I) Densitometric quantification of the Western blots. Data were normalized to actin and are the mean±SD. All data were analyzed by one-way ANOVA with Tukey's multiple comparison test. #p<0.05, ##p<0.01, ###p<0.001, ####p<0.0001 relative to vehicle control; * p<0.05, ** p<0.01, *** p<0.001, **** p<0.0001 relative to the 50 nM RSL3 treatment; ns, not significant.

FIGS. 5A-5D shows CBN prevents Ca²⁺ influx induced by oxytosis/ferroptosis in HT22 cells. (FIG. 5A) Cellular Ca²⁺ levels upon different treatment conditions of the cells for 16 h. Data were normalized to total protein/well and are the mean of 16 replicates per condition±SD. (FIG. 5B) Mitochondrial Ca²⁺ levels upon different treatment conditions of the cells for 16 h. Data were normalized to total protein/well and are the mean of 16 replicates per condition±SD. (FIG. 5C) Western blot data of MCU and actin (n=3−6). Protein levels were measured following different treatments of the cells for 16 h. (FIG. 5D) Densitometric quantification of the Western blots. Data were normalized to actin and are the mean±SD. All data were analyzed by one-way ANOVA with Tukey's multiple comparison test. #p<0.05, ##p<0.01, ###p<0.001, ####p<0.0001 relative to vehicle control; * p<0.05, ** p<0.01, *** p<0.001, **** p<0.0001 relative to the 50 nM RSL3 treatment.

FIGS. 6A-6H shows CBN preserves mitochondrial bioenergetics following induction of oxytosis/ferroptosis. (FIG. 6A) Mitochondrial oxygen consumption rate (OCR) profiles in HT22 cells after different treatments for 16 h. Data were normalized to total protein/well and are the mean of 20 replicates per condition±SD. (FIG. 6B) Graphs for basal respiration, maximal respiration, and ATP production in HT22 cells. (FIG. 6C) Mitochondrial oxygen consumption rate (OCR) profiles in primary cortical neurons after different treatments for 16 h. Data were normalized to total protein/well and are the mean of 20 replicates per condition±SD. (FIG. 6D) Graphs for basal respiration, maximal respiration, and ATP production in primary cortical neurons. (FIG. 6E) Mitochondrial membrane potential in HT22 cells after different treatments for 4 h. Data are the mean of 8-12 replicates per condition±SD. (FIG. 6F) Relative mtDNA copy number in HT22 cells after different treatments for 16 h. Data are the mean of 3 replicates per condition±SD. (FIG. 6G) Western blot data of ETC complex proteins (ATP5A, MTCO1, UQCRC2, SDHB, NDUFB8), TOM20, VDAC, and actin (n=3−6). Protein levels were measured following different treatments of the cells for 16 h. (FIG. 6H) Densitometric quantification of the Western blots. Data were normalized to TOM20 and are the mean±SD. All data were analyzed by one-way ANOVA with Tukey's multiple comparison test. #p<0.05, ##p<0.01, ###p<0.001, ####p<0.0001 relative to vehicle control; * p<0.05, ** p<0.01, *** p<0.001, **** p<0.0001 relative to the 50 nM RSL3 treatment; ns, not significant.

FIG. 7 shows mitochondrial membrane potential in different cell lines. The cells were treated with compounds for 4 hr.

FIGS. 8A-8H shows CBN stimulates mitochondrial biogenesis following induction of oxytosis/ferroptosis in HT22 cells. Representative fluorescent images of HT22 cells following treatment for 16 h: (FIG. 8A) 0.2% ethanol treatment as vehicle control, (FIG. 8B) 5 μM CBN treatment, (FIG. 8C) 50 nM RSL3 treatment, (FIG. 8D) 5 μM CBN pretreatment for 1 h followed by 50 nM RSL3 treatment. Mitochondria stained with MitoTracker (red); nuclei stained with Hoechst 33342 (blue). Enlarged images from the boxed areas are indicated. The micrographs show representative morphological characteristics of the cells under the different conditions with 4 experimental replicates per condition. Scale bar=200 μm. (FIG. 8E) Relative quantification of mitochondrial mass with MitoTracker in HT22 cells following different treatment conditions for 16 h. Data were normalized to total protein/well and are the means of 16 replicates per condition±SD. (FIG. 8F) Protein levels of VDAC relative to actin were measured following the different treatments of the HT22 cells for 16 h using immunoblotting. Data are the means of 3-6 replicates per condition±SD. (FIG. 8G) Western blot data of SIRT1, pAMPKα (Thr172), total AMPKα, PGC-1α, NRF1, TFAM, and actin (n=3−6). Protein levels were measured following different treatments of the cells for 16 h. (FIG. 8H) Densitometric quantification of the Western blots. Data were normalized to actin and are the mean±SD. All data were analyzed by one-way ANOVA with Tukey's multiple comparison test. #p<0.05, ##p<0.01, ###p<0.001, ####p<0.0001 relative to vehicle control; * p<0.05, ** p<0.01, *** p<0.001, **** p<0.0001 relative to the 50 nM RSL3 treatment; ns, not significant.

FIGS. 9A-9K shows CBN modulates mitochondrial fusion/fission dynamics following induction of oxytosis/ferroptosis in HT22 cells. Representative fluorescent images of HT22 mt-GFP cells following treatment for 16 h: (FIG. 9A) 0.2% ethanol treatment as vehicle control, (FIG. 9B) 5 μM CBN treatment, (FIG. 9C) 50 nM RSL3 treatment, (FIG. 9D) 5 μM CBN pretreatment for 1 h followed by 50 nM RSL3 treatment. Enlarged images from the boxed areas are indicated in the bottom panels. The micrographs show representative images of the morphological characteristics of the cells under the different conditions with 4 experimental replicates per condition. As visualized in FIG. 9A, the control HT22 cells contained tubular-shaped, elongated, and branched networks of mitochondria as previously reported. The cells treated with 5 μM CBN for 16 hr (FIG. 9B) appeared to have more widespread mitochondria with highly branched networks. In contrast, the cells treated with 50 nM RSL3 for 16 hr showed a large number of fragmented, shortened and globular/donut-shaped mitochondria (FIG. 9C), similar to what has been seen in stressed, pathologic, or aging/senescent neurons. Pretreatment of CBN for 1 hr followed by RSL3 co-incubation for 16 hr was able to reduce the number of fragmented mitochondria and maintain the tubular mitochondrial morphology and networks similar to the control cells (FIG. 9D). Scale bar=10 μm. (FIG. 9E) Mitochondrial network morphology analyses on micrographs of HT22 cells after different treatments: (FIG. 9E) mitochondrial footprint, (FIG. 9F) mitochondrial branch length, (FIG. 9G) mitochondrial summed branch lengths, (FIG. 9H) mitochondrial network branches. Data are the mean of 20-25 cells per condition±SD. (FIG. 9I) & (FIG. 9J) Western blot data of OPA1, MFN2, DRP1, MFF, and actin (n=3−6). Protein levels were measured following the different treatments of the cells for 16 h. (FIG. 9K) Densitometric quantification of the Western blots. Data were normalized to actin and are the mean±SD. All data were analyzed by one-way ANOVA with Tukey's multiple comparison test. #p<0.05, ##p<0.01, ###p<0.001, ####p<0.0001 relative to vehicle control; * p<0.05, ** p<0.01, *** p<0.001, **** p<0.0001 relative to the 50 nM RSL3 treatment; ns, not significant.

FIGS. 10A-10E shows CBN requires functional mitochondria for protection against oxytosis/ferroptosis in HT22 cells. (FIG. 10A & FIG. 10B) Representative fluorescent images of HT22 mt-GFP WT and mt-GFP/mCherry-Parkin cells following the different treatment conditions. GFP-labeled mitochondria (green), mCherry-Parkin (red). FCCP-induced mitophagy in mt-GFP/mCherry-Parkin cells but not in mt-GFP cells. The micrographs show representative images of the morphological characteristics of the cells under the different conditions with 12 experimental replicates per condition. Fluorescence microscopic images (FIG. 10A) illustrate that CBN exhibited strong and equivalent protection against RSL3-induced cell death in both cell lines. When both were pretreated with FCCP (5 μM) for 24 hr (FIG. 10B), the mt-GFP/mCherry-Parkin cells showed a partial removal of mitochondria as evidenced by fewer and compressed/clustered GFP-labeled mitochondria mostly surrounding the nucleus, indicative of widespread mitophagy. Scale bar=50 μm. (FIG. 10C, FIG. 10D & FIG. 10E) DNA-based cell viability analysis of HT22 mt-GFP or mt-GFP/mCherry-Parkin cells following the different treatment conditions. Data are the mean of 8-12 replicates per condition±SD. All data were analyzed by one-way ANOVA with Tukey's multiple comparison test. ####p<0.0001 relative to vehicle control; **** p<0.0001 relative to the 50 nM RSL3 treatment; \hat{ }\hat{ }\hat{ }\hat{ }p<0.0001 relative to the 5 μM FCCP treatment; ●●●●p<0.0001 relative to the FCCP (5 μM)+RSL3 (50 nM) treatment; ns, not significant.

FIG. 11 shows the western blotting of mitochondrial markers in HT22 mt-GFP and mt-GFP/mCherry-Parkin cell lines with/without FCCP induction.

FIG. 12 shows the proposed neuroprotective mechanisms of CBN against oxytosis/ferroptosis through directly targeting mitochondria to restore multiple key parameters of mitochondrial function. Oxytosis/ferroptosis induced by RSL3 (down arrows) causes an increase in mitochondrial oxidative stress and calcium overload, disturbance of mitochondrial membrane potential, and decrease in mitochondrial bioenergetics, biogenesis, and fusion/fission dynamics. CBN protects neuronal cells through counteracting all these six aspects of mitochondrial dysfunction (up arrows)

FIG. 13 shows the 1H NMR spectrum of CBN collected at 600 MHz in DMSO-D6 using 5 mm TXI probe.

FIG. 14 shows the GCMS chromatogram of CBN showing 99.9% purity.

FIG. 15 shows the GCMS chromatogram of H₂CBND showing 99.6% purity.

FIG. 16 shows the GCMS chromatogram of CBN—OAc showing 97.56% purity.

FIG. 17 shows the experimental design for Example 4.

FIG. 18 shows the results from the Barnes Maze test.

FIG. 19 shows the GCMS chromatogram of CBF showing 99.4% purity.

FIG. 20 shows the GCMS chromatogram of CBND showing 95.76% purity.

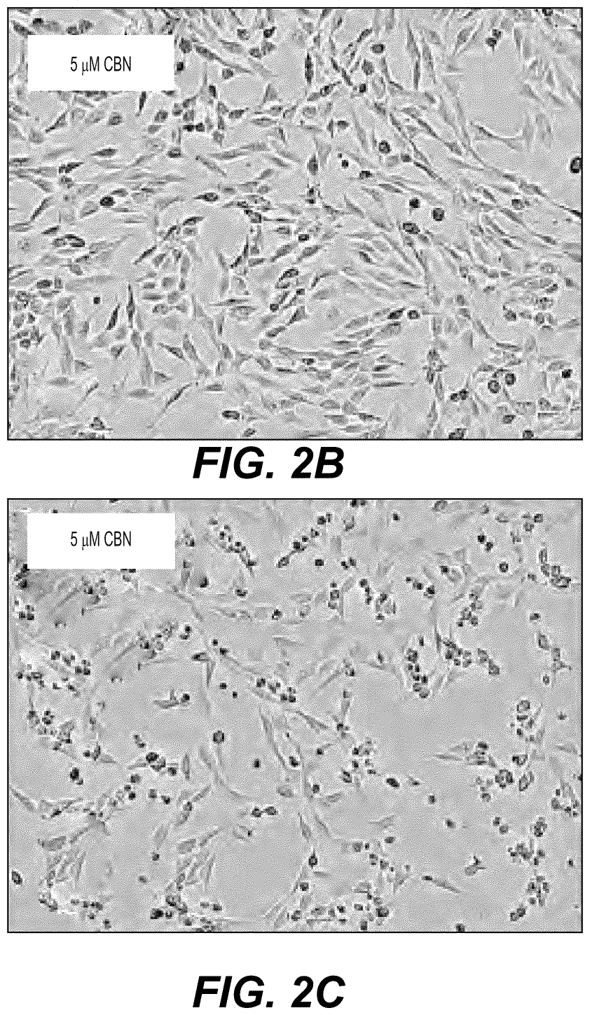

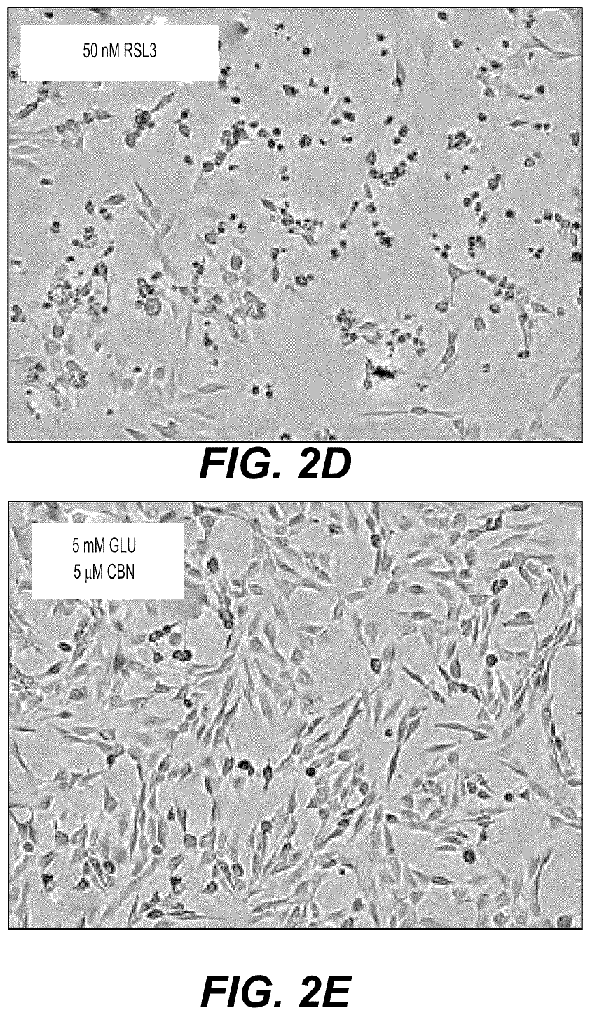

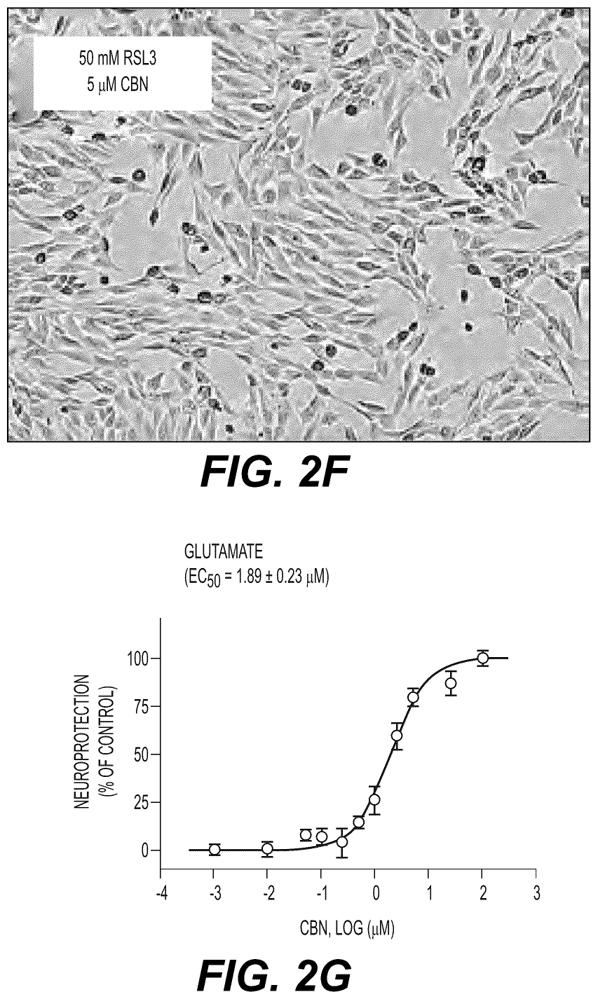

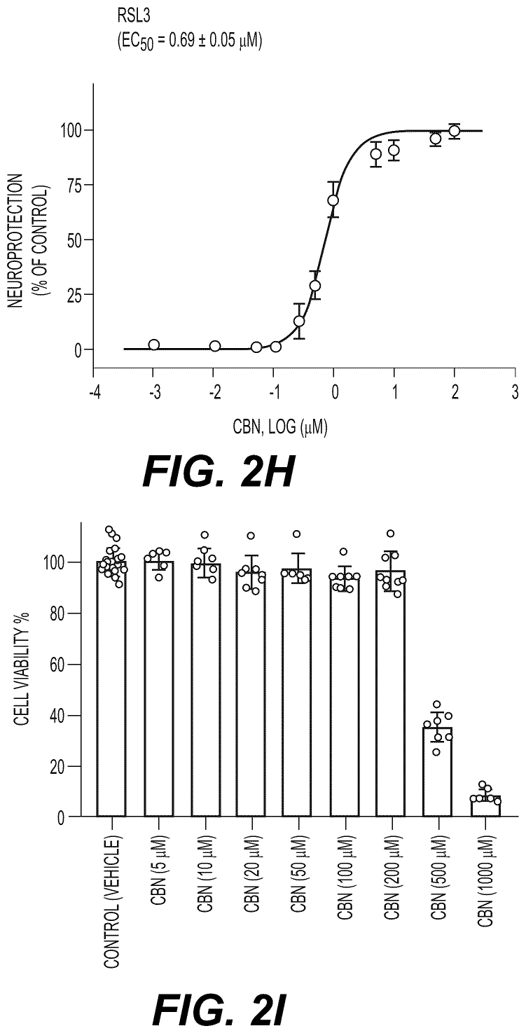

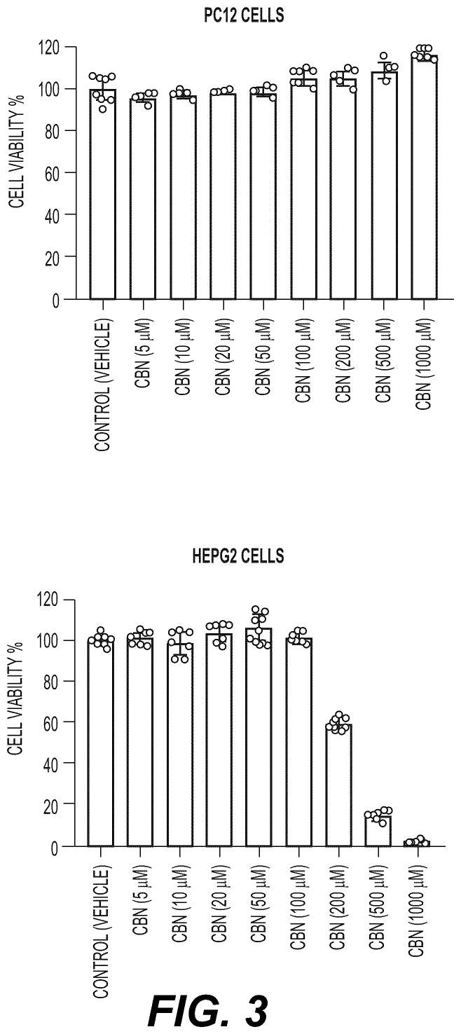

FIG. 2I shows the GCMS chromatogram of d8-THC showing 99.64% purity.

FIG. 22 shows the GCMS chromatogram of d9-THC showing 98.5% purity.

FIG. 23 shows the GCMS chromatogram of d10-THC showing 94.68% purity.

FIG. 24 shows the GCMS chromatogram of d6a, 10α-THC showing 98.49% purity.

FIG. 25 shows the GCMS chromatogram of D8(9)-iso-THC showing 94.38% purity.

FIG. 26 shows the GCMS chromatogram of D4(8)-iso-THC showing 88.59% purity.

FIG. 27 shows the GCMS chromatogram of D8(9)-iso-THCBF showing 77.71% purity.

FIG. 28 shows the GCMS chromatogram of H₂CBD showing 98% purity.

FIG. 29 shows the GCMS chromatogram of H₄CBD showing 84.95% purity.

FIG. 30 shows the GCMS chromatogram of H₄CBG showing 99.4% purity.

FIG. 31 shows the GCMS chromatogram of H₂CBND showing 99.64% purity.

FIG. 32 shows the GCMS chromatogram of HHC showing 99.82% purity.

FIG. 33 shows the GCMS chromatogram of CBNV showing 99.79% purity.

FIG. 34 shows the GCMS chromatogram of CBN—C1 showing 99.32% purity.

FIG. 35 shows the GCMS chromatogram of CBD(OAc)₂ showing 97.88% purity.

FIG. 36 shows the GCMS chromatogram of CBN—OAc showing 98.50% purity.

FIG. 37 shows the GCMS chromatogram of CBNV—OAc showing 98.52% purity.

FIG. 38 shows the GCMS chromatogram of 4-DI-CDND showing 99.04% purity.

### DETAILED DESCRIPTION

Provided in the present disclosure are methods of treating a neurodegenerative disease or condition in a subject in need thereof, the method including administering a therapeutically effective amount of a cannabinoid to the subject. Compared with other neuroprotectors and/or oxytosis/ferroptosis inhibitors, cannabinoids, such as cannabinol (CBN), have several advantages. For example, cannabinol is demonstrated to not only protect nerve cells from oxytosis/ferroptosis in a manner that is dependent on mitochondria but also does so independently of cannabinoid receptors. Specifically, CBN directly targets mitochondria and preserves key mitochondrial functions including redox regulation, calcium uptake, membrane potential, bioenergetics, biogenesis, and modulation of fusion/fission dynamics that are disrupted following induction of oxytosis/ferroptosis. The present disclosure describes methods of using mitochondrially-targeted cannabinoids, such as CBN, as oxytotic/ferroptotic inhibitors to rescue mitochondrial dysfunction.

Provided are methods of treating a neurodegenerative disease or condition in a subject, the method including administering a therapeutically effective amount of a cannabinoid to the subject. In some embodiments, the cannabinoid is an oxytosis/ferroptosis inhibitor. In some embodiments, the cannabinoid is a neuroprotector. For example, provided are methods of treating a neurodegenerative disease or condition in a subject that includes administering a therapeutically effective amount of cannabinol to the subject, where the cannabinol inhibits oxytosis/ferroptosis. For example, provided are methods of treating a neurodegenerative disease or condition in a subject that includes administering a therapeutically effective amount of cannabifuran to the subject, where the cannabifuran inhibits oxytosis/ferroptosis. For example, provided are methods of treating a neurodegenerative disease or condition in a subject that includes administering a therapeutically effective amount of tetrahydrocannabidiol to the subject, where the tetrahydrocannabidiol inhibits oxytosis/ferroptosis. For example, provided are methods of treating a neurodegenerative disease or condition in a subject that includes administering a therapeutically effective amount of tetrahydrocannabigerol to the subject, where the tetrahydrocannabigerol inhibits oxytosis/ferroptosis.

Also provided are methods of treating a disease or disorder associated with mitochondrial dysfunction associated with an aging brain in a subject, the method including administering a therapeutically effective amount of a cannabinoid to the subject. For example, provided are methods of treating a disease or disorder associated with mitochondrial dysfunction associated with an aging brain in a subject that includes administering a therapeutically effective amount of cannabinol (CBN) to the subject. For example, provided are methods of treating a disease or disorder associated with mitochondrial dysfunction associated with an aging brain in a subject that includes administering a therapeutically effective amount of cannabifuran to the subject. For example, provided are methods of treating a disease or disorder associated with mitochondrial dysfunction associated with an aging brain in a subject that includes administering a therapeutically effective amount of tetrahydrocannabidiol to the subject. For example, provided are methods of treating a disease or disorder associated with mitochondrial dysfunction associated with an aging brain in a subject that includes administering a therapeutically effective amount of tetrahydrocannabigerol CBN to the subject.

Also provided herein are methods of inhibiting oxytosis/ferroptosis in a subject, the method including administering a cannabinoid to the subject.

Unless otherwise defined, all technical and scientific terms used in this document have the same meaning as commonly understood by one of ordinary skill in the art to which the present application belongs. Methods and materials are described in this document for use in the present application; other, suitable methods and materials known in the art can also be used. The materials, methods, and examples are illustrative only and not intended to be limiting. All publications, patent applications, patents, sequences, database entries, and other references mentioned in this document are incorporated by reference in their entirety. In case of conflict, the present specification, including definitions, will control.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (for example, 1%, 2%, 3%, and 4%) and the sub-ranges (for example, 0.1% to 0.5%, 1.1% to 2.2%, and 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

The term “about,” as used in this disclosure, can allow for a degree of variability in a value or range, for example, within 5%, or within 1% of a stated value or of a stated limit of a range.

As used in this disclosure, the terms “a,” “an,” and “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

In the methods described in this disclosure, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, the term “cannabinoid” refers to a compound that is structurally related to the terpophenolic compounds metabolically produced by Cannabis sativa. Cannabinoids include the endocannabinoids (produced naturally in the body by humans and animals), the phytocannabinoids (found in Cannabis and some other plants), and synthetic cannabinoids. For example, a notable phytocannabinoid is Δ⁹⁽¹⁰⁾-tetrahydrocannabinol (Δ⁹⁽¹⁰⁾-THC), the primary psychoactive compound of Cannabis. Cannabidiol is another major constituent of the plant, representing up to 40% in extracts of the plant resin. There are at least 85 different cannabinoids isolated from Cannabis, which exhibit varied effects.

As used herein, the term “substantially free of” an ingredient(s) as provided throughout the disclosure is intended to mean that the composition or compound(s) contain less than about 0.1 wt % (percent by weight of the total weight of the composition or compound(s)), or insignificant or negligible amounts of said ingredient(s) unless specifically indicated otherwise. In some embodiments, the CBN used in the methods of the present disclosure is substantially free of THC, meaning that the CBN contains less than about 0.1 wt % THC. In some embodiments, the CBN used in the methods of the present disclosure is substantially free of Δ⁹⁽¹⁰⁾-THC, meaning that the CBN contains less than about 0.1 wt % Δ⁹⁽¹⁰⁾-THC.

In some embodiments, the cannabinoid is a neuroprotector. In some embodiments, the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

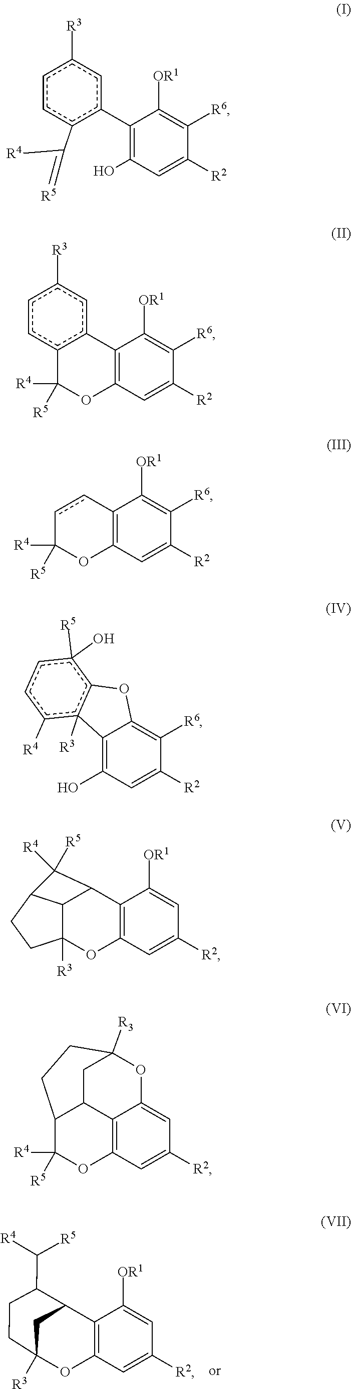

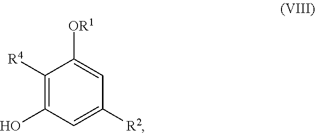

Or a pharmaceutically acceptable salt thereof, wherein: each [symbol] is independently a single bond or a double bond; R¹ is hydrogen, C₁-C₁₀ alkyl, C(═O)—(C₁-C₁₀ alkyl), or Si(C₁-C₁₀ alkyl)₃; R² is C₁-C₁₀ alkyl; R³ is C₁-C₆ alkyl optionally substituted with OH; R⁴ is C₁-C₁₀ alkyl or C₂-C₁₀ alkenyl; R⁵ is absent, C₁-C₁₀ alkyl or C₂-C₁₀ alkenyl; and R⁶ is hydrogen, C₁-C₆ alkyl, C(═O)(OH), or C(═O)—(C₁-C₆ alkyl).

In some embodiments, the cannabinoid is a compound of Formula (I). In some embodiments, the cannabinoid is a compound of Formula (II). In some embodiments, the cannabinoid is a compound of Formula (III). In some embodiments, the cannabinoid is a compound of Formula (IV). In some embodiments, the cannabinoid is a compound of Formula (V). In some embodiments, the cannabinoid is a compound of Formula (VI). In some embodiments, the cannabinoid is a compound of Formula (VII). In some embodiments, the cannabinoid is a compound of Formula (VIII).

In some embodiments, R¹ is hydrogen or C₁-C₁₀ alkyl. In some embodiment, R¹ is hydrogen. In some embodiments, R¹ is C₁-C₆ alkyl. In some embodiments, R¹ is methyl, ethyl, or propyl. In some embodiments, R¹ is C(═O)—(C₁-C₁₀ alkyl). In some embodiments, R¹ is C(═O)—(C₁-C₃ alkyl). In some embodiments, R¹ is Si(C₁-C₁₀ alkyl)₃. In some embodiments, R¹ is Si(C₁-C₃ alkyl)₃. In some embodiments, R¹ is Si(CH₃)₃.

In some embodiments, R² is C₁-C₆ alkyl. In some embodiments, R² is C₅ alkyl. In some embodiments, R² is C₃ alkyl.

In some embodiments, R³ is C₁-C₃ alkyl. In some embodiments, R³ is methyl. In some embodiments, R³ is C₁-C₃ alkyl substituted with OH. In some embodiments, R³ is CH₂OH.

In some embodiments, R⁴ is C₁-C₆ alkyl. In some embodiments, R⁴ is C₅-C₁₀ alkyl. In some embodiments, R⁴ is methyl, ethyl, or propyl. In some embodiments, R⁴ is methyl. In some embodiments, R⁴ is C₂-C₁₀ alkenyl. In some embodiments, R⁴ is C₄-C₈ alkenyl.

In some embodiments, R⁵ is absent. In some embodiments, R⁵ is C₁-C₆ alkyl. In some embodiments, R⁵ is C₅-C₁₀ alkyl. In some embodiments, R⁵ is methyl, ethyl, or propyl. In some embodiments, R⁵ is methyl. In some embodiments, R⁵ is C₂-C₁₀ alkenyl. In some embodiments, R⁵ is C₄-C₈ alkenyl.

In some embodiments, R⁶ is H. In some embodiments, R⁶ is C₁-C₃ alkyl. In some embodiments, R⁶ is methyl. In some embodiments, R⁶ is C(═O)(OH). In some embodiments, R⁶ is C(═O)—(C₁-C₆ alkyl). In some embodiments, R⁶ is C(═O)—(C₁-C₃ alkyl).

In some embodiments, the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), and combinations thereof. In some embodiments, the cannabinoid is selected from the group consisting of cannabinol, acetylcannabinol, dihydrocannabinodiol, iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

Exemplary cannabinoids that can be used in the methods of the present disclosure are described in Table 1.

TABLE 1
Cannabinoids

| Trivial Name | Structure | IUPAC name |
|---|---|---|
| Cannabichromene (CBC) | 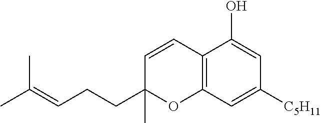 | 2-Methyl-2-(4-methylpent-3- enyl)-7-pentyl-5-chromenol |
| Cannabichromenic acid (CBCA) | 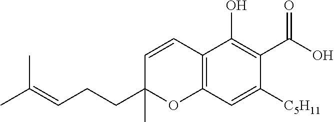 | 5-Hydroxy-2-methyl-2-(4- methylpent-3-enyl)-7- pentylchromene-6- carboxylic acid |
| Cannabichromenevarin (CBCV) | 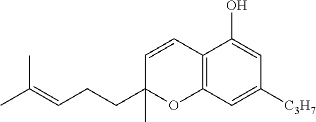 | 2-Methyl-2-(4-methylpent- 3-enyl)-7-propyl-5- chromenol |
| Cannabidiol (CBD) | 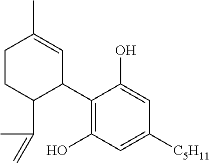 | 2-[(1R,6R)-6-Isopropenyl-3- methylcyclohex-2-en-1-yl]- 5-pentylbenzene-1,3-diol |
| Cannabidiolic acid (CBDA) | 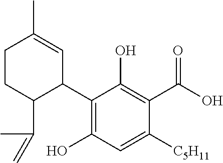 | (1′R,2′R)-2,6-Dihydroxy-5′- methyl-4-pentyl-2′-(prop-1- en-2-yl)-1′,2′,3′,4′- tetrahydro[1,1′-biphenyl]- 3-carboxylic acid |
| Cannabidivarol (CBDV) | 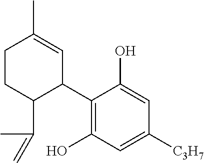 | 2-[(1R,6R)-6-Isopropenyl- 3-methylcyclohex-2-en-1- yl]-5-propylbenzene-1,3- diol |
| Cannabielsoin (CBE) | 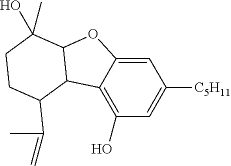 | (5aS,6S,9R,9aR)-6-methyl- 3-pentyl-9-prop-1-en-2-yl- 7,8,9,9a-tetrahydro-5aH- dibenzofuran-1,6-diol |
| Cannabifuran (CBF) | 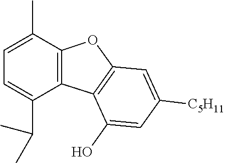 | 5-isopropyl-8-methyl-2- pentyl-9-oxa-4-fluorenol |
| Cannabigerol (CBG) | 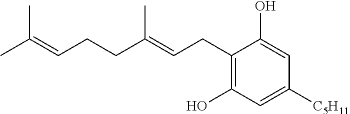 | 2-[(2E)-3,7-Dimethylocta- 2,6-dienyl]-5-pentyl- benzene-1,3-diol |
| Cannabigerolic acid (CBGA) | 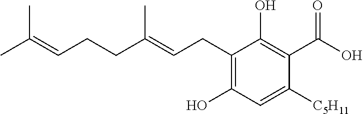 | 3-[(2E)-3,7-Dimethylocta- 2,6-dien-1-yl]-2,4- dihydroxy-6- pentylbenzoic acid |
| Cannabigerovarinol (CBGV) | 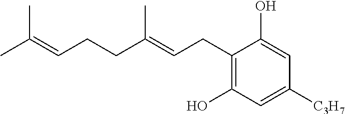 | 2-[(2E)-3,7-Dimethylocta- 2,6-dienyl]-5-propyl- benzene-1,3-diol |
| Cannabicyclol (CBL) | 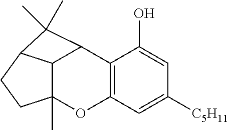 | (1aR-(1a alpha,3a alpha,8b alpha,8calpha))-1a,2,3,3a, 8b,8c-hexahydro-1,1,3a- trimethyl-6-pentyl-1H-4- oxabenzo(f)- cyclobut(cd)inden-8-ol |
| Cannabinol (CBN) | 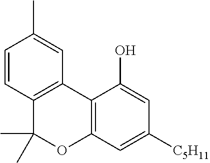 | 6,6,9-Trimethyl-3-pentyl- benzo[c]chromen-1-ol |
| Cannabinolic acid (CBNA) | 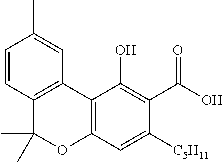 | 1-hydroxy-6,6,9-trimethyl-3- pentyl-benzo[c]chromene-2- carboxylic acid |
| Cannabivarol (CBNV) | 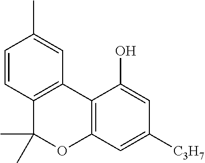 | 6,6,9-trimethyl-3-pentyl- benzo[c]chromen-1-ol |
| Acetylcannabinol (CBN-OAc) | 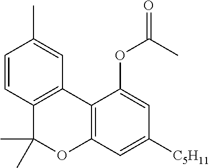 | 1-acetoxy-6,6,9-trimethyl-3- pentyl-benzo[c]chromene |
| Methoxycannabinol (CBN-OMe) | 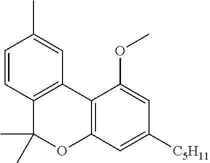 | 1-methoxy-6,6,9-trimethyl- 3-pentyl-benzo[c]chromene |
| Cannabinodiol (CBND) | 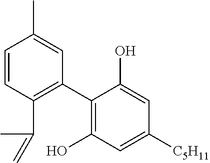 | 2,6-dihydroxy-4-pentyl-2′- isopropenyl-5′-methyl-1,1′- biphenyl |
| Cannabicitran (CBT) | 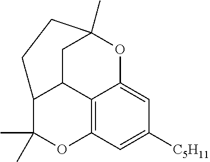 | 1,5,5-trimethyl-9-pentyl- 6,15-dioxa- tetracyclo[9.3.1.0^{4,13}.0^{7,12}]- pentadeca-7(12),8,10-triene |
| Dehydrocannabifuran (DHCBF) | 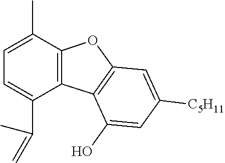 | 5-isopropenyl-8-methyl-2- pentyl-9-oxa-4-fluorenol |
| Dihydrocannabinodiol (H₂CBND) | 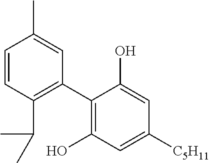 | 2,6-dihydroxy-4-pentyl-2′- isopropyl-5′-methyl-1,1′- biphenyl |
| Dihydrocannabidiol (H₂CBD) | 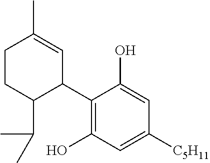 | 2-[(1R,6R)-6-Isopropyl-3- methylcyclohex-2-en-1-yl]- 5-pentylbenzene-1,3-diol |
| Tetrahydrocannabidiol (H₄CBD) | 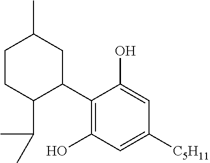 | 2-[(1S,6R)-6-Isopropyl-3- methylcyclohexyl]-5- pentylbenzene-1,3-diol |
| Δ⁸⁽⁹⁾-iso- tetrahydrocannabinol (Δ⁸⁽⁹⁾-iso-THC) | 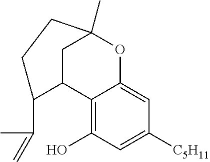 | 3,4,5,6-tetrahydro-2-methyl- 5-(1-methylethenyl)-9- pentyl-2,6-methano-2H-1- benzoxocin-7-ol |
| Δ⁴⁽⁸⁾-iso- tetrahydrocannabinol (Δ⁴⁽⁸⁾-iso-THC) | 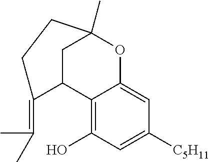 | 3,4,5,6-tetrahydro-2-methyl- 5-(1-methylethylidene)-9- pentyl-2,6-methano-2H-1- benzoxocin-7-ol |
| Δ⁴⁽⁵⁾-iso- tetrahydrocannabinol (Δ⁴⁽⁵⁾-iso-THC) | 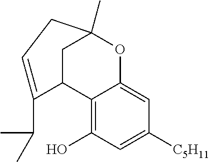 | 3,6-dihydro-2-methyl-5-(1- methylethyl)-9-pentyl-2,6- methano-2H-1-benzoxocin- 7-ol |
| Hexahydrocannabinol (HHC) | 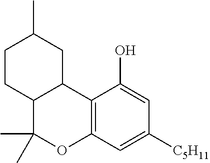 | 6,6,9-trimethyl-3-pentyl- 6a,7,8,9,10,10a-hexahydro- 6H-benzo[c]chromen-1-ol |
| Δ⁸⁽⁹⁾- tetrahydrocannabinol (Δ⁸⁽⁹⁾-THC) | 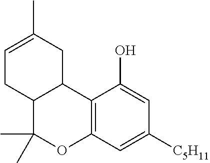 | 6,6,9-trimethyl-3-pentyl- 6a,7,10,10a-tetrahydro-6H- benzo[c]chromen-1-ol |
| Δ⁹⁽¹⁰⁾- tetrahydrocannabinol (Δ⁹⁽¹⁰⁾-THC) | 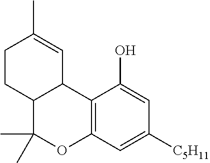 | 6,6,9-trimethyl-3-pentyl- 6a,7,8,10a-tetrahydro-6H- benzo[c]chromen-1-ol |
| Δ⁹⁽¹⁰⁾- tetrahydrocannabinolic acid (Δ⁹⁽¹⁰⁾-THCA) | 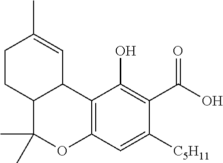 | (6aR,10aR)-1-Hydroxy- 6,6,9-trimethyl-3-pentyl- 6a,7,8,10a-tetrahydro-6H- benzo[c]chromene-2- carboxylic acid |
| Δ⁹⁽¹⁰⁾- tetrahydrocannabinovarol (Δ⁹⁽¹⁰⁾-THCV) | 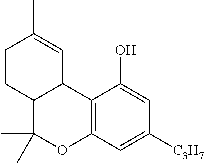 | 6,6,9-Trimethyl-3-propyl- 6a,7,8,10a-tetrahydro-6H- benzo[c]chromen-1-ol |
| Δ^{10(10a)}- tetrahydrocannabinol (Δ^{10(10a)}-THC) | 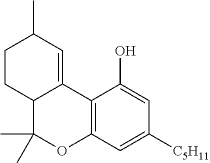 | 6,6,9-trimethyl-3-pentyl- 6a,7,8,9-tetrahydro-6H- benzo[c]chromen-1-ol |
| Δ^{6a(10a)}- tetrahydrocannabinol (Δ^{6a(10a)}-THC) | 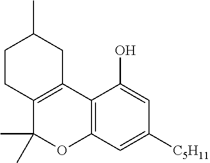 | 6,6,9-trimethyl-3-pentyl- 7,8,9,10-tetrahydro-6H- benzo[c]chromen-1-ol |
| iso-hexahydrocannabinol (iso-HHC) | 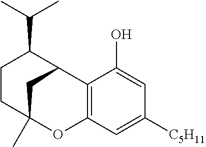 | (2α,5α,6α)-(−)-3,4,5,6- tetrahydro-2-methyl-5-(1- methylethyl)-9-pentyl- 2,6-methano-2H-1- benzoxocin-7-ol |
| 11-hydroxycannabinol (11-OH-CBN) | 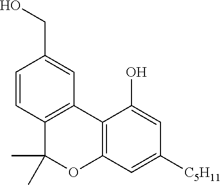 | 9-(hydroxymethyl)-6,6- dimethyl-3-pentyl-6H- benzo[c]chromen-1-ol |
| 4-desisopropenyl- cannabinodiol (4-DI- CBND) | 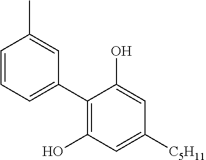 | 3′-methyl-4-pentyl- [1,1′-biphenyl]-2,6-diol |
| Abnormal Cannabidiol (Abn-CBD) | 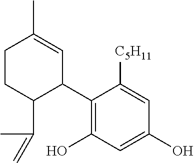 | 4-[(1R,6R)-3-methyl-6- (1-methylethenyl)-2- cyclohexen-1-yl]-5- pentyl-1,3-benzenediol |
| Abnormal Cannabivarol (Abn-CBNV) | 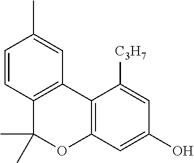 | 6,6,9-trimethyl-1- propyl-6H- benzo[c]chromen-3-ol |
| Diacetylcannabidiol (CBD-(OAc)₂), | 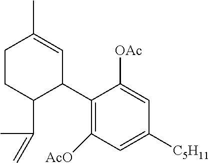 | 5′-methyl-4-pentyl-2′- (prop-1-en-2-yl)- 1′,2′,3′,4′-tetrahydro- [1,1′-biphenyl]-2,6-diyl diacetate |
| Cannabiorcinol (CBN-C₁), | 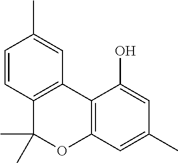 | 3-methyl-6,6,9- trimethyl-6H- benzo[c]chromen-1-ol |
| Cannabinol ethyl (CBN-C₂), | 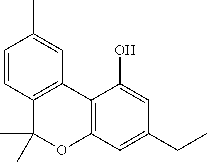 [SMILES: CCc1cc(O)c2c(c1)OC(C)(C)c1ccc(C)cc1-2] | 3-ethyl-6,6,9-trimethyl- 6H-benzo[c]chromen-1- ol |
| Cannabibutol (CBN-C₄), | 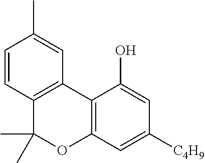 | 3-butyl-6,6,9-trimethyl- 6H-benzo[c]chromen-1- ol |
| Cannabihexol (CBN-C₆) | 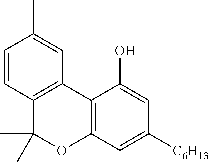 | 3-hexyl-6,6,9-trimethyl- 6H-benzo[c]chromen-1- ol |
| Cannabiphorol (CBN-C₇) | 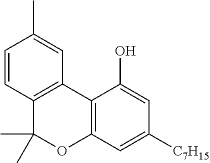 | 3-heptyl-6,6,9-trimethyl- 6H-benzo[c]chromen-1- ol |
| Acetylcannabivarol (CBNV-OAc) | 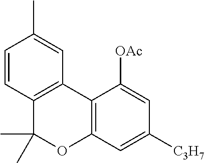 | 6,6,9-trimethyl-3-propyl- 6H-benzo[c]chromen-1- yl acetate |
| Δ8(9)-iso- tetrahydrocannabifuran (Δ⁸⁽⁹⁾-iso-THCBF) | 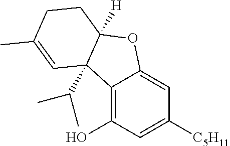 | (5aR,9aR)-9a-isopropyl- 8-methyl-3-pentyl- 5a,6,7,9a-tetra- hydrodibenzo[b,d]furan- 1-ol |
| Dihydrocannabielsoin (H₂CBE) | 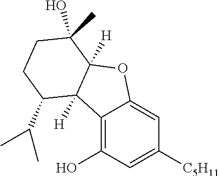 | (5aS,6S,9S,9aR)-9- isopropyl-6-methyl-3- pentyl-5a,6,7,8,9,9a- hexahydrodi- benzo[b,d]furan-1,6-diol |
| Tetrahydrocannabigerol (H₄CBG) | 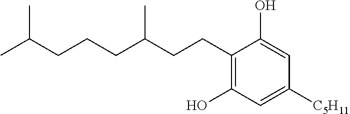 | 2-(3,7-dimethyloctyl)-5- pentylbenzene-1,3-diol |
| Olivetol | 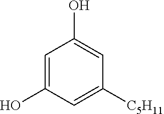 | 5-pentyl-1,3-benzenediol |

In some embodiments, the cannabinoid used in the methods of the present disclosure has high purity (e.g., a purity of at least about 90%, at least about 95%, at least about 98%, or at least about 99%). For example, the cannabinoid can be at least 95%, 95.5%, 96%, 96.5%, 97%, 97.5%, 98%, 98.5%, 99%, 99.5%, 99.6%, 99.7%, 99.8%, 99.9%, or 99.99% pure. In some embodiments, the cannabinoid has a purity of at least about 95%, at least about 95.5%, at least about 96%, at least about 96.5%, at least about 97%, at least about 97.5%, at least about 98%, at least about 98.5%, at least about 99%, at least about 99.5%, at least about 99.6%, at least about 99.7%, at least about 99.8%, or at least about 99.9%. In some embodiments, the cannabinoid has a purity of at least about 99%. In some embodiments, the cannabinoid has a purity of at least about 99.5%. In some embodiments, the cannabinoid has a purity of at least about 99.9%.

In some embodiments, the cannabinoid is cannabinol and the cannabinol has a purity of at least about 95%, at least about 95.5%, at least about 96%, at least about 96.5%, at least about 97%, at least about 97.5%, at least about 98%, at least about 98.5%, at least about 99%, at least about 99.5%, at least about 99.6%, at least about 99.7%, at least about 99.8%, or at least about 99.9%. In some embodiments, the cannabinol has a purity of at least about 99%. In some embodiments, the cannabinol has a purity of at least about 99.5%. In some embodiments, the cannabinol has a purity of at least about 99.9%.

In some embodiments, the cannabinoid is cannabifuran and the cannabifuran has a purity of at least about 95%, at least about 95.5%, at least about 96%, at least about 96.5%, at least about 97%, at least about 97.5%, at least about 98%, at least about 98.5%, at least about 99%, at least about 99.5%, at least about 99.6%, at least about 99.7%, at least about 99.8%, or at least about 99.9%. In some embodiments, the cannabifuran has a purity of at least about 99%. In some embodiments, the cannabifuran has a purity of at least about 99.5%. In some embodiments, the cannabifuran has a purity of at least about 99.9%.

In some embodiments, the cannabinoid is tetrahydrocannabidiol and the tetrahydrocannabidiol has a purity of at least about 95%, at least about 95.5%, at least about 96%, at least about 96.5%, at least about 97%, at least about 97.5%, at least about 98%, at least about 98.5%, at least about 99%, at least about 99.5%, at least about 99.6%, at least about 99.7%, at least about 99.8%, or at least about 99.9%. In some embodiments, the tetrahydrocannabidiol has a purity of at least about 99%. In some embodiments, the tetrahydrocannabidiol has a purity of at least about 99.5%. In some embodiments, the tetrahydrocannabidiol has a purity of at least about 99.9%.

In some embodiments, the cannabinoid is tetrahydrocannabigerol and the tetrahydrocannabigerol has a purity of at least about 95%, at least about 95.5%, at least about 96%, at least about 96.5%, at least about 97%, at least about 97.5%, at least about 98%, at least about 98.5%, at least about 99%, at least about 99.5%, at least about 99.6%, at least about 99.7%, at least about 99.8%, or at least about 99.9%. In some embodiments, the tetrahydrocannabigerol has a purity of at least about 99%. In some embodiments, the tetrahydrocannabigerol has a purity of at least about 99.5%. In some embodiments, the tetrahydrocannabigerol has a purity of at least about 99.9%.

In some embodiments, the cannabinoid used in the methods of the present disclosure contains about 0.5 wt % THC or less, such as about 0.45 wt %, about 0.4 wt %, about 0.35 wt %, about 0.3 wt %, about 0.25 wt %, about 0.2 wt %, about 0.15 wt %, about 0.1 wt % THC, or less. In some embodiments, the cannabinoid contains less than the legal limit of THC, such as less than 0.3 wt % THC. In some embodiments, the cannabinoid is substantially free of THC. In some embodiments, the THC is Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ^{10(10a)}-tetrahydrocannabinol (Δ^{10(10a)}-THC), or Δ^{6a(10a)}-tetrahydrocannabinol (Δ^{6a(10a)}-THC), or combinations thereof. In some embodiments, the cannabinoid contains about 0.5 wt % or less of Δ⁹⁽¹⁰⁾-THC. In some embodiments, the cannabinoid contains less than the legal limit of Δ⁹⁽¹⁰⁾-THC, such as less than 0.3 wt % Δ⁹⁽¹⁰⁾-THC. In some embodiments, the cannabinoid is substantially free of Δ⁹⁽¹⁰⁾-THC. Advantageously, the cannabinoids used in the methods of the present disclosure contain THC (e.g., Δ⁹⁽¹⁰⁾-THC) in such an insignificant amount that the psychoactive effects of certain forms of THC (e.g., Δ⁹⁽¹⁰⁾-THC) are diminished or are completely absent when a subject is administered the cannabinoid. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

In some embodiments, the cannabinoid used in the methods of the present disclosure has a purity of at least about 95%, at least about 95.5%, at least about 96%, at least about 96.5%, at least about 97%, at least about 97.5%, at least about 98%, at least about 98.5%, at least about 99%, at least about 99.5%, at least about 99.6%, at least about 99.7%, at least about 99.8%, or at least about 99.9% and contains about 0.5 wt % THC or less, such as about 0.45 wt %, about 0.4 wt %, about 0.35 wt %, about 0.3 wt %, about 0.25 wt %, about 0.2 wt %, about 0.15 wt %, about 0.1 wt % THC, or less. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

Typically, cannabinoids act on cannabinoid receptors on cells that repress neurotransmitter release in the brain. However, it has surprisingly been found that in the methods of the present disclosure, the cannabinoid inhibits oxytosis/ferroptosis independently of cannabinoid receptors. In some embodiments, the cannabinoid is cannabinol and the cannabinol inhibits oxytosis/ferroptosis independently of cannabinoid receptors. In some embodiments, the cannabinoid is cannabifuran and the cannabifuran inhibits oxytosis/ferroptosis independently of cannabinoid receptors. In some embodiments, the cannabinoid is tetrahydrocannabidiol and the tetrahydrocannabidiol inhibits oxytosis/ferroptosis independently of cannabinoid receptors. In some embodiments, the cannabinoid is tetrahydrocannabigerol and the tetrahydrocannabigerol inhibits oxytosis/ferroptosis independently of cannabinoid receptors.

The cannabinoid used in the methods of the present disclosure can be administered in an amount of about 1 mg/kg/day to about 50 mg/kg/day. For example, the cannabinoid can be administered in an amount of about 5 mg/kg/day to about 30 mg/kg/day, or about 10 mg/kg/day to 20 mg/kg/day. In some embodiments, the cannabinoid is administered in an amount of about 10 mg/kg/day to 20 mg/kg/day. In some embodiments, the cannabinoid is administered in an amount of about 10 mg/kg/day. In some embodiments, the cannabinoid is administered in an amount of about 20 mg/kg/day.

In some embodiments, the cannabinoid is cannabinol and the cannabinol is administered in an amount of about 1 mg/kg/day to about 50 mg/kg/day, or about 5 mg/kg/day to about 30 mg/kg/day, or about 10 mg/kg/day to 20 mg/kg/day. For example, the cannabinol is administered in an amount of about 10 mg/kg/day to 20 mg/kg/day. In some embodiments, the cannabinol is administered in an amount of about 10 mg/kg/day. In some embodiments, the cannabinol is administered in an amount of about 20 mg/kg/day.

In some embodiments, the cannabinoid is cannabifuran and the cannabifuran is administered in an amount of about 1 mg/kg/day to about 50 mg/kg/day, or about 5 mg/kg/day to about 30 mg/kg/day, or about 10 mg/kg/day to 20 mg/kg/day. For example, the cannabifuran is administered in an amount of about 10 mg/kg/day to 20 mg/kg/day. In some embodiments, the cannabifuran is administered in an amount of about 10 mg/kg/day. In some embodiments, the cannabifuran is administered in an amount of about 20 mg/kg/day.

In some embodiments, the cannabinoid is tetrahydrocannabidiol and the tetrahydrocannabidiol is administered in an amount of about 1 mg/kg/day to about 50 mg/kg/day, or about 5 mg/kg/day to about 30 mg/kg/day, or about 10 mg/kg/day to 20 mg/kg/day. For example, the tetrahydrocannabidiol is administered in an amount of about 10 mg/kg/day to 20 mg/kg/day. In some embodiments, the tetrahydrocannabidiol is administered in an amount of about 10 mg/kg/day. In some embodiments, the tetrahydrocannabidiol is administered in an amount of about 20 mg/kg/day.

In some embodiments, the cannabinoid is tetrahydrocannabigerol and the tetrahydrocannabigerol is administered in an amount of about 1 mg/kg/day to about 50 mg/kg/day, or about 5 mg/kg/day to about 30 mg/kg/day, or about 10 mg/kg/day to 20 mg/kg/day. For example, the tetrahydrocannabigerol is administered in an amount of about 10 mg/kg/day to 20 mg/kg/day. In some embodiments, the tetrahydrocannabigerol is administered in an amount of about 10 mg/kg/day. In some embodiments, the tetrahydrocannabigerol is administered in an amount of about 20 mg/kg/day.

The methods disclosed herein can include treating a neurodegenerative disease or condition in a subject. In some embodiments, the subject exhibits one or more risk factors or symptoms associated with a neurodegenerative disease or condition or the development of a neurodegenerative disease or condition. In some embodiments, the neurodegenerative disease or condition is an age-associated neurodegenerative disease or condition.

Examples of neurodegenerative diseases or conditions include, but are not limited to, Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

The methods disclosed herein include treating a disease or disorder associated with mitochondrial dysfunction associated with an aging brain in a subject. In some embodiments, the subject exhibits one or more risk factors or symptoms associated with a disease or disorder associated with mitochondrial dysfunction associated with an aging brain or the development of a disease or disorder associated with mitochondrial dysfunction associated with an aging brain.

In some embodiments, the disease or disorder associated with mitochondrial dysfunction associated with an aging brain is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis. In some embodiments, the disease or disorder associated with mitochondrial dysfunction associated with an aging brain is selected from the group consisting of Parkinson's disease, Alzheimer's disease, prion disease, a motor neuron disease (MND), amyotrophic lateral sclerosis (ALS), and Huntington's disease (HD).

In some embodiments, the subject is a mammal. In some embodiments, the subject is a human.

The methods disclosed herein include administering a cannabinoid to a subject, where administration of the cannabinoid to the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics. In some embodiments, the methods disclosed herein include administering cannabinol to a subject, where this administration of the cannabinol to the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics. In some embodiments, the methods disclosed herein include administering cannabifuran to a subject, where this administration of the cannabifuran to the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics. In some embodiments, the methods disclosed herein include administering tetrahydrocannabidiol to a subject, where this administration of the tetrahydrocannabidiol to the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics. In some embodiments, the methods disclosed herein include administering tetrahydrocannabigerol to a subject, where this administration of the tetrahydrocannabigerol to the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics.

In some embodiments, the methods disclosed herein include treating a subject, where treating the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal ß-amyloid in neuronal cells, and regulation of mitochondrial dynamics.

In some embodiments, the methods disclosed herein include inhibiting oxytosis/ferroptosis in a subject, where inhibiting oxytosis/ferroptosis in the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics.

The methods of the present disclosure can result in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics. Any method known to those skilled in the art can be used to assess the results of the methods of the present disclosure. In some embodiments, the results are determined using assays performed using a hippocampal nerve cell line lacking CB1 and CB2 receptors.

Also provided are methods of protecting nerve cells from oxytosis/ferroptosis in a subject, the method including administering cannabinol to the subject. Also provided are methods of protecting nerve cells from oxytosis/ferroptosis in a subject, the method including administering cannabifuran to the subject. Also provided are methods of protecting nerve cells from oxytosis/ferroptosis in a subject, the method including administering tetrahydrocannabidiol to the subject. Also provided are methods of protecting nerve cells from oxytosis/ferroptosis in a subject, the method including administering tetrahydrocannabigerol to the subject.

Also provided are dosage forms containing a cannabinoid, such as the cannabinoids disclosed herein. In some embodiments, the dosage form contains a pharmaceutically acceptable excipient and a cannabinoid. In some embodiments, the cannabinoid is cannabinol. In some embodiments, the cannabinoid is cannabifuran. In some embodiments, the cannabinoid is tetrahydrocannabidiol. In some embodiments, the cannabinoid is tetrahydrocannabigerol.

The cannabinoid included in the dosage form of the present disclosure can be in an amount of about 5 mg to about 5000 mg. For example, the cannabinoid in the dosage form can be in an amount of about 5 mg to about 5000 mg, about 50 mg to about 2000 mg, about 100 mg to about 1000 mg, about 250 mg to about 2500 mg, about 500 mg to about 1800 mg, or about 800 mg to about 1700 mg. In some embodiments, the cannabinoid in the dosage form is in an amount of about 800 mg to about 1700 mg. In some embodiments, the cannabinoid in the dosage form is in an amount of about 850 mg. In some embodiments, the cannabinoid in the dosage form is in an amount of about 1650 mg. For example, the dosage form can be provided such that the subject is administered the cannabinoid in an amount of about 5 mg to about 5000 mg per day, particularly about 5, about 25, about 50, about 100, about 200, about 300, about 400, about 450, about 500, about 550, about 600, about 700, about 750, about 800, about 850, about 900, about 1000, about 1100, about 1200, about 1300, about 1500, about 1600, about 1650, about 1700, about 1800, about 2000, about 2500, about 3000, about 3500, about 4000, about 4500, or about 5000 mg per day of the cannabinoid.

In some embodiments, the cannabinoid is cannabinol and the cannabinol is in the dosage form in an amount of about 5 mg to about 5000 mg, about 50 mg to about 2000 mg, about 100 mg to about 1000 mg, about 250 mg to about 2500 mg, about 500 mg to about 1800 mg, or about 800 mg to about 1700 mg. For example, the cannabinol is in the dosage form in an amount of about 800 mg to about 1700 mg. In some embodiments, the cannabinol is in the dosage form in an amount of about 850 mg. In some embodiments, the cannabinol is in the dosage form in an amount of about 1650 mg.

In some embodiments, the cannabinoid is cannabifuran and the cannabifuran is in the dosage form in an amount of about 5 mg to about 5000 mg, about 50 mg to about 2000 mg, about 100 mg to about 1000 mg, about 250 mg to about 2500 mg, about 500 mg to about 1800 mg, or about 800 mg to about 1700 mg. For example, the cannabifuran is in the dosage form in an amount of about 800 mg to about 1700 mg. In some embodiments, the cannabifuran is in the dosage form in an amount of about 850 mg. In some embodiments, the cannabifuran is in the dosage form in an amount of about 1650 mg.

In some embodiments, the cannabinoid is tetrahydrocannabidiol and the tetrahydrocannabidiol is in the dosage form in an amount of about 5 mg to about 5000 mg, about 50 mg to about 2000 mg, about 100 mg to about 1000 mg, about 250 mg to about 2500 mg, about 500 mg to about 1800 mg, or about 800 mg to about 1700 mg. For example, the tetrahydrocannabidiol is in the dosage form in an amount of about 800 mg to about 1700 mg. In some embodiments, the tetrahydrocannabidiol is in the dosage form in an amount of about 850 mg. In some embodiments, the tetrahydrocannabidiol is in the dosage form in an amount of about 1650 mg.

In some embodiments, the cannabinoid is tetrahydrocannabigerol and the tetrahydrocannabigerol is in the dosage form in an amount of about 5 mg to about 5000 mg, about 50 mg to about 2000 mg, about 100 mg to about 1000 mg, about 250 mg to about 2500 mg, about 500 mg to about 1800 mg, or about 800 mg to about 1700 mg. For example, the tetrahydrocannabigerol is in the dosage form in an amount of about 800 mg to about 1700 mg. In some embodiments, the tetrahydrocannabigerol is in the dosage form in an amount of about 850 mg. In some embodiments, the tetrahydrocannabigerol is in the dosage form in an amount of about 1650 mg.

The dosage form of the present disclosure may be administered on a regimen of 1 to 4 times per day, including once, twice, three times, and four times per day.

The term “pharmaceutically acceptable excipient” refers to an excipient for administration of a pharmaceutical agent, such as a cannabinoid (e.g., cannabinol, cannabifuran, tetrahydrocannabidiol, or tetrahydrocannabigerol) described herein. The term refers to any pharmaceutical excipient that may be administered without undue toxicity. Excipients include carriers, solvents, stabilizers, adjuvants, diluents, etc.

Suitable excipients can be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants (e.g., ascorbic acid), chelating agents (e.g., EDTA), carbohydrates (e.g., dextrin, hydroxyalkylcellulose, and/or hydroxyalkylmethylcellulose), stearic acid, liquids (e.g., oils, water, saline, glycerol and/or ethanol) wetting or emulsifying agents, pH buffering substances, and the like. Liposomes are also included within the definition of pharmaceutically acceptable excipients.

### Examples

Example 1: Cannabinol as Oxytosis/ferroptosis Inhibitors Abbreviations used below are as follows: AD, Alzheimer's disease; PD, Parkinson's disease; HD, Huntington's disease; CNS, central nervous system; mt, mitochondria; DNA, deoxyribonucleic acid; ETC, electron transport chain; OXPHOS, oxidative phosphorylation; ROS, reactive oxygen species; ΔΨm, mitochondrial membrane potential; PGC-1α, peroxisome proliferator-activated receptor-γ coactivator 1α; AMPK, adenosine monophosphate-activated protein kinase; SIRT1, sirtuin-1; NRF1, nuclear respiratory factor 1; TFAM, mitochondrial transcription factor A; OPA1, optic atrophy protein 1; MFN2, mitofusin 2; DRP1, dynamin-related protein 1; MFF, mitochondrial fission factor; Nrf2, nuclear erythroid 2-related factor; ATF4, activating transcription factor 4; HO-1, heme oxygenase-1; SOD2, superoxide dismutase 2; HSP60, mitochondrial chaperone heat shock protein 60; Xc-, cysteine/glutamate antiporter; GSH, glutathione; GPX4, glutathione peroxidase 4; VDAC, voltage-dependent anion channel; TOM20, translocase of the outer membrane subunit 20; MCU, mitochondrial calcium uniporter; GFP, green fluorescent protein; CB1/CB2, cannabinoid receptors 1 and 2; NADPH, nicotinamide adenine dinucleotide phosphate; ATP, adenosine triphosphate; OCR, oxygen consumption rate; TEAC, Trolox equivalent antioxidant capacity; WT, wild type; qPCR, quantitative polymerase chain reaction; NMR, nuclear magnetic resonance; HR-MS, high-resolution mass spectrometry; CBN, cannabinol; THC, tetrahydrocannabinol; CBD, cannabidiol; ABTS, 2,2′-azino-bis(3-ethylbenzothiazoline-6-sulfonic acid); FCCP, carbonyl cyanide-4-(trifluoromethoxy)phenylhydrazone; MTT, 3−(4,5-dimethylthiazol-2-yl)-2,5-diphenyltetrazolium bromide; EC₅₀, half maximal effective concentration.

### Material and Methods

Chemicals and Reagents: All solvents and reagents were purchased from commercial sources and were used without further purification. Cannabinol (CBN) was synthesized in-house according to the procedure disclosed in U.S. application Ser. No. 18/403,509, filed Jan. 3, 2024, and described elsewhere herein. Olivetol, citral, n-butylamine, iodine, Na₂SO₃, ethyl acetate, petroleum ether, toluene, glutamate, RSL3, FCCP, Trolox, ABTS, potassium persulfate, ferrous sulfate, ferrozine, HEPES, complet protease inhibitor cocktail, PhosSTOP phosphatase inhibitor cocktail, and Nonidet P-40 were from Sigma-Aldrich (Saint Louis, MO). Dowex 50WX8 (Cat #AAAL13921) was from VWR (Radnor, PA). Ferrostatin, MitoQ, and STY-BODIPY (Cat #27089) were from Cayman Chemical (Ann Arbor, MI). Seahorse XFe96 FluxPak (Cat #102416) and Seahorse XF Cell Mito Stress Test Kit (Cat #103015) were from Agilent Technologies (Santa Clara, CA). Calcium indicators Rhod-2 AM (Cat #R1244) and Fluo-4 AM (Cat #F14201), CellROX Green Reagent (Cat #C₁₀₄₄₄), MitoSOX Red Mitochondrial Superoxide Indicator (Cat #M36008), C₁₁-BODIPY Lipid Peroxidation Sensor (Cat #D3861), MitoTracker Orange CM−H₂TMRos (Cat #M7511), NucBlue Live Cell Stain Hoechst 33342 Reagent (Cat #R37605), CyQUANT Direct Cell Proliferation Assay Kit (Cat #C35011), Pierce BCA Protein Assay Kit (Cat #23227), Cell Lysis Buffer (Cat #FNN0011), SuperSignal West Pico PLUS Chemiluminescent Substrate (Cat #34578), HyperSep Silica Cartridge (Cat #60108-712) were from Thermo Fisher Scientific (Waltham, MA). JC-10 Mitochondrial Membrane Potential Assay Kit (Cat #ab112134) was from Abcam (Burlingame, CA). Criterion XT Bis-Tris Protein Gradient Gel (Ca #3450125), XT MOPS Running Buffer (Cat #1610788), Trans-Blot Turbo RTA Transfer Kit (Cat #1704275), Precision Plus Protein Standards (Cat #1610373) were from Bio-Rad (Hercules, CA).

General Instrumental Analysis: Optical absorbance and fluorescence were measured on a SpectraMax M5 Multi-Mode microplate reader (Molecular Devices, San Jose, CA).

Microscopy. Brightfield, phase contrast, and fluorescence microscopic images were acquired on an IX51 inverted microscope (Olympus Corporation, Tokyo, Japan) with an INFINITY3 monochrome CCD camera (Teledyne Lumenera, Ontario, Canada). Super-resolution microscopic images were acquired on a Zeiss LSM 880 rear port laser scanning confocal and Airyscan FAST microscope (Carl-Zeiss, Oberkochen, Germany). Image processing and analysis were performed with microscope software packages ZEN Black and ImageJ/Fiji.

Mass Spectrometry (MS): High-resolution mass spectrometric data were obtained on a Thermo Q-Exactive Quadrupole-Orbitrap mass spectrometer in positive mode. Samples were diluted with a 1:1 mixture of methanol and water containing 0.1% formic acid and then introduced by direct electrospray infusion. Accurate masses of all analytes were obtained from the pseudo-molecule [M+H]+ and were within 5 ppm mass error. Full MS scans were recorded for the 150-750 m/z range. MS/MS fragmentation was achieved by higher-energy collisional dissociation (HCD) at normalized collision energy settings between 10 and 30%.

Nuclear magnetic resonance (NMR): 1H, ¹³C and 2D NMR data were collected at 298 K on a 600 MHz Bruker Avance III spectrometer fitted with a 1.7 mm triple resonance cryoprobe with z-axis gradients using TopSpin 3.6.0. NMR spectra were referenced to the residual solvent signal (δH 7.26, δC77.2 for chloroform-d) with chemical shifts reported in 8 units (ppm). Resonance multiplicities are denoted s, d, t, q, m, and br for singlet, doublet, triplet, quartet, multiplet, and broad, respectively. 2D HSQC and HMBC NMR spectra were collected using modified versions of the Bruker pulse sequences hsqcedetgpsisp2.3 and hmbcctetgpl3ndsp, which incorporated an ASAP module to enable faster data acquisition (E. Kupče, R. Freeman, Fast multidimensional NMR by polarization sharing, Magn. Reson. Chem. 45(1) (2007) 2-4). Spectral widths were 12 ppm for the 1H dimensions, 160 ppm for the HSQC ¹³C dimension and 250 ppm for the HMBC ¹³C dimension. For the HSQC spectra 8 scans and 256 t1 increments were used. For the HMBC spectra 32 scans and 512 t1 increments were used. Spectra were analyzed using Mnova.

Cell Culture. HT22 mouse hippocampal nerve cells were cultured in high-glucose Dulbecco's modified Eagle's medium (DMEM) (Invitrogen, Cat #11995065, Carlsbad, CA) supplemented with 10% fetal bovine serum (FBS) (Invitrogen, Carlsbad, CA) and 1% antibiotics including penicillin and streptomycin (Invitrogen Cat #10378016, Carlsbad, CA). Cell cultures were incubated at 37° C. in a fully humidified atmosphere containing 10% CO₂.

SH-SY5Y neuroblastoma cells were cultured in 1:1 (v/v) DMEM/F12 medium (Invitrogen, Cat #11330032, Carlsbad, CA) supplemented with 1% non-essential amino acids (Invitrogen, Cat #11140050), 10% FBS, and 1% antibiotics including penicillin and streptomycin. Cell cultures were incubated at 37° C. in a fully humidified atmosphere containing 10% CO₂.

BV2 microglial cells were cultured in low glucose DMEM supplemented with GlutaMAX (Invitrogen, Cat #10567014, Carlsbad, CA), 1 mM sodium pyruvate, 10% FBS, and 1% antibiotics including penicillin and streptomycin. Cell cultures were incubated at 37° C. in a fully humidified atmosphere containing 10% CO₂.

Embryonic Cortical Neuron Extraction. Primary cortical neurons were prepared from embryonic day 17 Sprague-Dawley rat pups and used at 7 days in vitro as previously described (7 DIV). Neurons were dissociated from the cortex and maintained in Neurobasal-A medium (Invitrogen, Cat #10888022, Carlsbad, CA) supplemented with B-27 Supplement (Invitrogen, Cat #17504044, Carlsbad, CA), 2 mM glutamine, and 1% antibiotics including penicillin and streptomycin. Cell cultures were incubated at 37° C. in a fully humidified atmosphere containing 10% CO₂. All animal procedures were approved by the Salk Institute's Institutional Animal Care and Use Committee.

Generation of Stable HT22 mt-GFP and mt-GFP/mCherry-Parkin Cell Lines. The procedure was carried out as described. The mitochondrially targeted green fluorescent protein (GFP) plasmid was transfected into HT22 cells using GenJet In Vitro DNA Transfection Reagent (Ver. II) (SignaGen laboratories, Cat #SL100489, Frederick, MD) and stably fluorescent cell colonies were then isolated. The resulting stable line was transfected with a plasmid expressing mCherry-Parkin and a similar procedure was performed to isolate cells stably co-expressing both constructs.

Cell Protein Extraction. HT22 cells were seeded onto 10 cm dishes at a density of 400,000 cells/dish in DMEM supplemented with 10% FBS and 1% antibiotics, and then exposed to the desired treatments. For total protein extracts, HT22 cells were washed with ice-cold phosphate buffered saline (PBS) and lysed with cell extraction buffer containing 10 mM Tris, pH 7.4, 100 mM NaCl, 1 mM EDTA, 1 mM EGTA, 1 mM NaF, 20 mM Na₄P₂O₇, 2 mM Na₃VO₄, 1% Triton X-100, 10% glycerol, 0.1% sodium dodecyl sulfate (SDS), 0.5% sodium deoxycholate, a protease inhibitor cocktail, and a phosphatase inhibitor cocktail followed by centrifugation (14000 g) for 30 min at 4° C. For nuclear extracts, HT22 cells were washed with ice-cold PBS, incubated with a nuclear fractionation buffer containing 10 mM HEPES, pH 7.9, 10 mM KCl, 0.1 mM EDTA, 0.1 mM EGTA, 1 mM DTT, 1 mM Na₃VO₄, a protease inhibitor cocktail, and a phosphatase inhibitor cocktail on ice for 15 min, then Nonidet P-40 at a final concentration of 0.6% was added to the buffer. Cells were vortexed, and the nuclei pelleted by centrifugation (1000 g) for 10 min at 4° C. Nuclear proteins were extracted by sonication of the nuclear pellet in nuclear fractionation buffer followed by centrifugation (14000 g) for 30 min at 4° C. The resulting supernatants of total/nuclear protein extracts were stored at −80° C. until analysis. Concentrations of the harvested proteins were determined by the BCA protein assay.

Immunoblotting. Western blots were carried out as follows. Laemmli sample buffer with 2% β-mercaptoethanol was added to the samples and then boiled for 5 min prior to SDS-PAGE. Equal amounts of cellular protein for each sample (10 μg per lane) were resolved by 4-12% gradient Criterion XT Precast Bis-Tris Gels (Bio-Rad, Hercules, CA) and transferred onto PVDF membranes with a Trans-Blot Turbo System (Bio-Rad, Hercules, CA). The membranes were blocked with 5% skim milk in TBST (20 mM Tris buffer, pH 7.5, 0.5 M NaCl, 0.1% Tween 20) for 1 hr at room temperature and incubated overnight at 4° C. with the diluted primary antibody in 5% BSA in TBST. After washes with TBST, the membranes were incubated with horseradish peroxidase-conjugated goat anti-rabbit or anti-mouse secondary antibodies (Bio-Rad, Hercules, CA) diluted 1:5000 in 5% skim milk in TBST. For all primary antibodies, the same membrane was re-probed for β-actin or an antiserum reacting with the total protein. After additional washing, protein bands were detected using the SuperSignal West Pico PLUS Chemiluminescent Substrate, and visualized with a ChemiDoc MP Imaging System (Bio-Rad, Hercules, CA). Band density was quantified densitometrically with Image Lab software (Bio-Rad, Hercules, CA). Relative protein expression was normalized to ß-actin band density. Each Western blot was performed three to six times with independent protein samples (n=3−6).

The primary antibodies used were from the following suppliers: antibodies against OPA1 (Cat #80471, 1:3000), MFN2 (Cat #9482, 1:3000), DRP1 (Cat #8570, 1:3000), MFF (Cat #84580, 1:3000), TOM20 (Cat #42406, 1:3000), VDAC (Cat #4661, 1:3000), SIRT1 (Cat #9475, 1:3000), total AMPKα (Cat #5831, 1:3000), phosphoThr172-AMPKα (Cat #2535, 1:1000), SOD2 (Ca #13141, 1:3000), GPX4 (Cat #52455, 1:3000), MCU (Cat #14997, 1:3000), HSP60 (Cat #12165, 1:3000), β-actin (Cat #4970, 1:3000) were from Cell Signaling Technology (Danvers, MA); antibodies against TFAM (Ca #ab131607, 1:3000), NRF1 (Cat #ab175932, 1:3000), total OXPHOS (Cat #ab ab110413, 1:3000) were from Abcam (Cambridge, MA); antibodies against ATF4 (Cat #sc-200, 1:1000), Nrf2 (Cat #sc-13032, 1:1000) were from Santa Cruz Biotechnology (Dallas, TX); anti-HO-1 (Cat #SPA-896, 1:3000) was from Stressgen (Victoria, BC, Canada); anti-PGC-1a (Cat #AB3242, 1:3000) was from Sigma-Aldrich (Saint Louis, MO). Horseradish peroxidase-conjugated secondary antibodies (Cat #1706515, and 1706516) were from Bio-Rad (Hercules, CA).

Oxytosis Assay. HT22 cells were seeded at 3,000 cells/well in 96-well tissue culture plates in DMEM plus 10% FBS and 1% antibiotics. After 24 hr of plating, the cells were pretreated with different concentrations of the test compounds or a vehicle control for 1 hr followed by coincubation with 5 mM glutamate to initiate the cell death cascade. After 16 hr of treatment, cell viability was measured by the 3−(4,5-dimethylthiazol-2-yl)-2,5-diphenyltetrazolium bromide (MTT) assay. Optical absorbance was measured at 570 nm on a SpectraMax M5 microplate reader. Samples were analyzed in eight to sixteen wells per independent experiment (n=8-16). Results were presented as the percentage of the controls with vehicle alone. Results were verified by visual inspection of the cells under a microscope and shown in Table 2.

Ferroptosis Assay: HT22 cells were seeded at 3,000 cells/well in 96-well tissue culture plates in DMEM plus 10% FBS and 1% antibiotics. After 24 hr of plating, the cells were pretreated with different concentrations of test compounds or a vehicle control for 1 hr followed by coincubation with 50 nM RSL3 to induce the cell death cascade. After 16 hr of treatment, cell viability was measured by the MTT assay. Optical absorbance was measured at 570 nm on a SpectraMax M5 microplate reader. Samples were analyzed in eight to sixteen wells per independent experiment (n=8-16). Results were presented as the percentage of the controls with vehicle alone. Results were verified by visual inspection of the cells under a microscope and shown in Table 2.

TABLE 2
Results from Oxytosis and Ferroptosis Assays

| | | Observed | Observed | Observed | | |
|---|---|---|---|---|---|---|
| | | EC₅₀ | EC₅₀ | EC₅₀ | | |
| | Abbreviated | glutamate | erastin | RSL3 | | Purity |
| Compound Name | Name | (μM) | (μM) | (μM) | Source | (%) |
| Cannabichromene | CBC | 2.5 | 8.7 | 1.1 | BayMedica | 98 |
| Cannabidiol | CBD | 2.3 | 8.8 | 0.86 | GenCanna | 99 |
| Cannabielsoin | CBE | >10 | >10 | >10 | Cayman | 95 |
| Cannabifuran | CBF | 0.22 | 0.31 | 0.15 | FloraWorks | 99 |
| Cannabigerol | CBG | 5.5 | 10.8 | 3.2 | GenCanna | 99 |
| Cannabicyclol | CBL | 1.1 | 3.3 | 0.77 | Cayman | 98 |
| Cannabinol | CBN | 1.3 | 2.5 | 0.39 | FloraWorks | 99.9 |
| Cannabinodiol | CBND | 14.2 | 15.4 | 7.7 | FloraWorks | 95 |
| Cannabicitran | CBT | >10 | >10 | >10 | BayMedica | 98 |
| Δ⁸⁽⁹⁾- | D8-THC | 0.8 | 2.6 | 0.8 | FloraWorks | 99 |
| tetrahydrocannabinol | | | | | | |
| Δ⁹⁽¹⁰⁾- | D9-THC | 6.1 | 9.4 | 4.1 | FloraWorks | 99 |
| tetrahydrocannabinol | | | | | | |
| Δ^{10(10a)}- | D10-THC | 0.53 | 0.62 | 0.13 | FloraWorks | 93 |
| tetrahydrocannabinol | | | | | | |
| Δ^{6a(10a)}- | D6a,10a- | 1.5 | 1.8 | 0.7 | FloraWorks | 98 |
| tetrahydrocannabinol | THC | | | | | |
| Δ⁸⁽⁹⁾⁻iso- | D8(9)-iso- | 0.86 | 2.1 | 0.33 | FloraWorks | 94 |
| tetrahydrocannabinol | THC | | | | | |
| Δ⁴⁽⁸⁾-iso- | D4(8)-iso- | 0.46 | 1.5 | 0.34 | FloraWorks | 88 |
| tetrahydrocannabinol | THC | | | | | |
| Δ⁸⁽⁹⁾-iso- | D8(9)-iso- | 1.8 | 5.1 | 1.4 | FloraWorks | 80 |
| tetrahydrocannabifuran | THCBF | | | | | |
| Cannabidiolic acid | CBDA | >10 | >10 | 9.3 | Cayman | 95 |
| Cannabigerolic acid | CBGA | >10 | >10 | >10 | Cayman | 97 |
| Cannabinolic acid | CBNA | >10 | >10 | >10 | Cayman | 98 |
| Δ⁹⁽¹⁰⁾- | THCA | >10 | >10 | >10 | Cayman | 98 |
| tetrahydrocannabinolic | | | | | | |
| acid | | | | | | |
| Cannabidivarol | CBDV | 2.1 | 3.8 | 1.0 | BayMedica | 98 |
| Δ⁹⁽¹⁰⁾- | THCV | 1.6 | 3.4 | 0.78 | BayMedica | 95 |
| tetrahydrocannabinovarol | | | | | | |
| 8,9-Dihydrocannabidiol | H₂CBD | 2.8 | 4.8 | 1.1 | FloraWorks | 98 |
| Tetrahydrocannabidiol | H₄CBD | 0.2 | 0.3 | 0.4 | FloraWorks | 95 |
| Dihydrocannabielsoin | H₂CBE | 7.5 | 9.2 | 3.5 | Cayman | 98 |
| Tetrahydrocannabigerol | H₄CBG | 0.7 | 0.7 | 0.4 | FloraWorks | 99 |
| 8.9- | H₂CBND | 4 | 5.7 | 2.4 | FloraWorks | 99 |
| Dihydrocannabinodiol | | | | | | |
| Hexahydrocannabinol | HHC | 0.5 | 0.5 | 0.3 | FloraWorks | 99 |
| iso-hexahydrocannabinol | iso-HHC | 1.4 | 2.4 | 0.8 | Cayman | 98 |
| Abnormal Cannabivarol | Abn-CBNV | 5.4 | 9.5 | 4.9 | Cayman | 98 |
| Abnormal Cannabidiol | Abn-CBD | 7.5 | 8 | 3.9 | Cayman | 98 |
| Cannabiphorol | CBN-C₇ | 2.4 | 3.8 | 2 | Cayman | 98 |
| Cannabihexol | CBN-C₆ | 1.6 | 2 | 0.9 | Cayman | 98 |
| Cannabibutol | CBN-C₄ | 1.7 | 2.1 | 1.1 | Cayman | 98 |
| Cannabivarinol | CBNV or | 2.8 | 4.6 | 1.8 | FloraWorks | 99 |
| | CBN-C₃ | | | | | |
| Cannabinol ethyl | CBN-C₂ | 4 | 5.6 | 1.8 | Cayman | 98 |
| Cannabiorcinol | CBN-C₁ | 3.8 | 6.9 | 2.2 | FloraWorks | 99 |
| Diacetylcannabidiol | CBD-(OAc)₂ | 3.8 | 9.1 | 2.4 | FloraWorks | 97 |
| Acetylcannabinol | CBN-OAc | 1.6 | 1.9 | 1.3 | FloraWorks | 98 |
| Acetylcannabivarol | CBNV-OAc | 2.2 | 4.0 | 1.6 | FloraWorks | 98 |
| Methoxycannabinol | CBN-OMe | >10 | >10 | >10 | Cayman | 95 |
| 11-hydroxycannabinol | 11-OH-CBN | 2.1 | 4.7 | 1.3 | Cayman | 98 |
| 4-desisopropenyl- | 4-DI-CBND | 7.7 | 9.6 | 4.2 | FloraWorks | 98 |
| cannabinodiol | | | | | | |
| Olivetol | | — | — | — | Sigma | 95 |
| | | | | | Aldrich | |

Reactive Oxygen Species Measurement. HT22 cells were seeded onto 96-well black walled plates at a density of 5,000 cells/well in DMEM supplemented with 10% FBS and 1% antibiotics. After the desired treatments, whole cell ROS and mitochondrial superoxide ROS were detected with CellROX Green reagent (Ex/Em=485/520 nm) and MitoSOX Red reagent (Ex/Em=510/580 nm), respectively. Experiments were performed according to the manufacturer's instructions. Fluorescence was measured on a SpectraMax M5 microplate reader. Data were normalized for total protein/well. Each condition was analyzed in sixteen wells per independent experiment (n=16). Results were presented as the percentage of the controls with vehicle alone. Results were verified by live-cell imaging under a fluorescence microscope.

Lipid Peroxidation MeasurementsHT22 cells were seeded onto 96-well black walled plates at a density of 3,000 cells/well in DMEM supplemented with 10% FBS and 1% antibiotics. After the desired treatments, cells were labeled with 2.5 μM C₁₁-BODIPY 581/591 (oxidized form Ex/Em=488/520 nm) at 37° C. for 2 hr. Experiments were performed according to the manufacturer's instructions. Fluorescence was measured on a SpectraMax M5 microplate reader. Data were normalized for total protein/well. Each condition was analyzed in twelve wells per independent experiment (n=12). Results were presented as the percentage of the controls with vehicle alone. Results were verified by live-cell imaging under a fluorescence microscope.

For the cell-free, liposome-based assay, STY-BODIPY (1.5 μM) and liposomes of egg-PC (1 mM) (Avanti Polar Lipids Inc, Cat #840051P, AL) in TBS (pH 7.4) were added to an opaque 96-well plate. This was followed by the addition of test compounds (10 μM). The plate was incubated for 30 min at 37° C. and then vigorously mixed for 5 min. The autoxidation was initiated by the addition of V-70 (0.5 mM) (Fujifilm Wako, Cat #001-70078, Japan), followed by additional mixing for 5 min. Data were acquired at Ex/Em=488/518 nm every 15 min at 37° C. on a SpectraMax M5 microplate reader. Data were transformed into [ox-STY-BODIPY] by taking the raw fluorescent values of the saturated curve of control DMSO and dividing them by the initial concentration of reduced STY-BODIPY (1.5 M). Samples were analyzed in quadruplicate in four independent experiments (n=4).

Trolox Equivalent Antioxidant Capacity (TEAC) Assay. Briefly, an aqueous 2,2′-azino-bis(3-ethylbenzothiazoline-6-sulfonic acid) (ABTS) solution (7 mM) was treated with potassium persulfate (K₂S₂O₈) (2.5 mM) overnight at room temperature in the dark and diluted in water to an OD value of 0.7 at 734 nm. 190 μL of the diluted ABTS solution was added to a 96-well plate containing 10 μL of test compounds or Trolox at the desired concentrations. The plate was vigorously mixed and incubated for 4 min at room temperature in the dark. Optical absorbance was measured at 734 nm on a SpectraMax M5 microplate reader. The decrease in absorbance of the ABTS radical cation indicates the presence of antioxidants. Samples were analyzed in eight wells per independent experiment (n=8). The TEAC values of each sample were presented as the percentage of the standard Trolox at 5 μM.

Iron (Fe²⁺) Binding Assay. Ferrous iron binding was measured by the ferrozine method. Briefly, test samples at the desired concentrations were mixed with 5 μM ferrous sulfate (FeSO₄) in 100 μL of 50 mM HEPES, pH 7.5 in a 96-well plate. After 2 min, 50 μL of 5 mM ferrozine was added and the optical absorbance at 562 nm was measured on a SpectraMax M5 microplate reader. Each sample was analyzed in eight wells per independent experiment (n=8). Iron binding capacities were presented as the percentage of the vehicle controls without (100%) or with iron (0%).

Total GSH Measurements. HT22 cells were seeded onto 35 mm dishes at a density of 70,000 cells/dish in DMEM supplemented with 10% FBS and 1% antibiotics. After the desired treatments, the cells were scraped into ice-cold PBS and the cell pellets collected by centrifugation (500 g). The pellets were resuspended in PBS and 10% sulfosalicylic acid was added at a final concentration of 3.3% to the cells. Proteins were pelleted by centrifugation and the supernatant neutralized. Total GSH was determined by the recycling assay based on the reduction of 5,5-dithiobis(2-nitrobenzoic acid) with glutathione reductase and NADPH and normalized to protein recovered from the acid-precipitated pellet by treatment with 0.2 N NaOH at 37° C. overnight and measured by the BCA protein assay (Pierce, Rockford, IL, USA). Samples were analyzed in four dishes per independent experiment (n=4).

Calcium Measurements. HT22 cells were seeded onto 96-well black walled plates at a density of 5,000 cells/well in DMEM supplemented with 10% FBS and 1% antibiotics. After the desired treatments, Ca²⁺ levels were detected with Fluo-4 AM (Ex/Em=494/516 nm) and Rhod-2 AM (Ex/Em=552/581 nm) calcium indicator dyes specific to cytosol and mitochondria, respectively. Fluorescence was measured on a SpectraMax M5 microplate reader. Experiments were performed according to the manufacturer's instructions. Data were normalized for total protein/well. Each condition was analyzed in sixteen wells per independent experiment (n=16). Results were presented as the percentage of the controls with vehicle alone. Results were verified by live-cell imaging under a fluorescence microscope.

Seahorse XF Analysis. The assay procedure was previously described [30]. Cellular oxygen consumption rates (OCR) were assayed with a XF Cell Mito Stress Test Kit using a Seahorse XFe96 Extracellular Flux Analyzer (Seahorse Bioscience, North Billerica, MA). Complete Seahorse XF DMEM assay medium was supplemented with 10 mM glucose, 1 mM pyruvate and 2 mM L-glutamine, at pH 7.4. Mitochondrial ETC inhibitors were used at the following concentrations: 1.5 μM oligomycin, 2 μM FCCP, and 0.5 μM of a 1:1 mixture of rotenone and antimycin A. Analyses were conducted using Wave software and XF Report Generators (Agilent Technologies). The sensor cartridge for the XFe analyzer was hydrated overnight at 37° C. before the experiment. OCR data were normalized for total protein/well. Each condition was analyzed in 20-40 wells per independent experiment (n=20-40).

For HT22 cells, 3,000 cells/well were seeded onto the Seahorse XFe96 plates under normal culture condition as described above. The next day, cells were pretreated with CBN at the desired concentrations for 1 hr followed by addition of RSL3 (50 nM) and coincubation for 16 hr. Immediately before the assay, the culture medium in the plates was replaced with complete Seahorse XF DMEM assay medium. The plates were incubated for 1 hr at 37° C. prior to the XF Cell Mito Stress tests according to the manufacturer's instructions.

For primary cortical neurons, the cells were seeded onto poly-L-ornithine coated Seahorse XFe96 plates at a density of 20,000 cells/well under the culture condition as described above and incubated for two days. On day 3, the cells were pretreated with CBN at the desired concentrations for 1 hr followed by addition of RSL3 (50 nM) and coincubation for 16 hr. Immediately before the assay, the culture medium in plates was replaced with complete Seahorse XF DMEM assay medium. The plates were incubated for 1 hr at 37° C. prior to the XF Cell Mito Stress tests according to the manufacturer's instructions.

Mitochondrial Membrane Potential Assay. Cells seeded at 5,000 cells/well onto 96-well black walled plates were treated with test compounds at the desired concentrations under the culture condition described above. The mitochondrial uncoupler FCCP at 2 μM was used as a reference control. After the desired treatments for 4 hr, the cells were subjected to a JC-10 mitochondrial membrane potential assay according to the manufacturer's instruction. Fluorescence intensities (Ex/Em=490/525 nm, and Ex/Em=540/590 nm) of each well were monitored on a SpectraMax M5 microplate reader. The ratio of fluorescence intensity (590/525 nm) was used to determine the mitochondrial membrane potential (ΔΨm). Decreasing ratios indicate mitochondrial membrane depolarization. Samples were analyzed in eight to sixteen wells per independent experiment (n=8-16). Results were verified by live-cell imaging under a fluorescence microscope.

Quantitative Reverse Transcription PCR (qPCR) Analysis. HT22 cells were seeded onto 60 mm dishes at a density of 140,000 cells/dish in DMEM supplemented with 10% FBS and 1% antibiotics. After the desired treatments, total RNA was extracted using the RNeasy Mini Kit (Qiagen, Cat #74104, Hilden, Germany). cDNA was synthesized using SuperScript III First-Strand Synthesis System for qPCR (Invitrogen, Cat #18080051, Carlsbad, CA). Amplification was performed with TaqMan Fast Advanced Master Mix (ThermoFisher Scientific, Cat #4444556, Carlsbad, CA) using a QuantStudio 3 Real-Time PCR Systems (ThermoFisher Scientific, CA). The primer pairs were used as follows: CNR1 (Forward, 5′-AAGTCGATCTTAGACGGCCTT-3′; Reverse, 5′-TCCTAATTTGGATGCCATGTCTC-3′); CNR2 (Forward, 5′-ACGGTGGCTTGGAGTTCAAC-3′; Reverse, 5′-GCCGGGAGGACAGGATAAT-3′). The expression of the GAPDH gene was measured for normalization. The mouse Mitochondrial DNA Copy Number Kit was used (Detroit R&D, Cat #MCN3, Detroit, MI) according to the manufacturer's instructions. Relative fold change was calculated using the 2^{(-ΔΔCt)} method. Samples were analyzed in three dishes per independent experiment (n=3).

MitoTracker Assay. HT22 cells were seeded onto 96-well black walled plates at a density of 5,000 cells/well in DMEM supplemented with 10% FBS and 1% antibiotics. After the desired treatments, MitoTracker Orange CM−H₂TMRos (Ex/Em=554/576 nm) and Hoechst 33342 (Ex/Em=360/460 nm) dyes were added to the cells and incubated under the same culture condition for 2 hr. Fluorescence was measured on a SpectraMax M5 microplate reader. Experiments were performed according to the manufacturer's instructions. Data were normalized for total protein/well. Each condition was analyzed in sixteen wells per independent experiment (n=16). Results were presented as the percentage of the controls with vehicle alone. Results were verified by live-cell imaging under a fluorescence microscope.

Mitochondrial Network Morphology Analysis. HT22 mt-GFP cells were seeded at 20,000 cells/well on glass coverslips in 24-well plates in DMEM supplemented with 10% FBS and 1% antibiotics. The following day, the cells were treated with the test compounds for the desired period of time. After rinsing with PBS, the cells on glass coverslips were fixed with 4% paraformaldehyde (pH 7.4) for 10 min at 37° C. After additional PBS rinses, the coverslips were mounted onto glass microslides with Fluoro-Gel (Electron Microscopy Sciences, Cat #17985-10). Z-stack images of fixed cells were acquired on a Zeiss LSM 880 rear port laser scanning confocal and Airyscan FAST microscope with ZEN Black software to trace and render cells in 3D. The mitochondrial network morphology parameters (n=20-25 cells/condition) were scored and analyzed with the MiNA module of ImageJ/Fiji software.

CyQUANT Cell Proliferation Assay. HT22 mt-GFP or mt-GFP/mCherry-Parkin cells were seeded onto 96-well black walled plates at a density of 3,000 cells/well in DMEM supplemented with 10% FBS and 1% antibiotics. After overnight incubation, the cells were pretreated with 5 μM FCCP or vehicle for 24 hr followed by the addition of test compounds or vehicle for an additional 16 hr. After the desired treatments, CyQUANT (Ex/Em=508/560 nm) reagents were added to the cells and incubated for 1 hr. The experiments were performed according to the manufacturer's instructions. Fluorescence was measured on a SpectraMax M5 microplate reader. Each condition was analyzed in eight to twelve wells per independent experiment (n=8-12). Results were presented as the percentage of the controls with vehicle alone. Results were verified by live-cell imaging under a fluorescence microscope.

Statistical Analysis. Data was presented as the mean±SD. The half maximal effective concentration (EC₅₀) was determined from sigmoidal dose response curves with four-parameter regression. The data were analyzed by one-way ANOVA with Tukey's multiple comparison post hoc test or Student's t test where appropriate. P values less than 0.05 were considered statistically significant (* p<0.05, ** p<0.01, *** p<0.001, and **** p<0.0001). Analyses were performed using Excel and GraphPad Prism

### Results

Cannabinoids and derivatives thereof are prepared in U.S. application Ser. No. 18/403,509, filed Jan. 3, 2024, and incorporated herein by reference.

The identity of CBN was confirmed by high-resolution nuclear magnetic resonance (NMR) and mass spectrometry (MS) analyses. 1H NMR (600 MHZ, CDCl₃) δ 8.18 (s, 1H), 7.15 (d, J=7.9 Hz, 1H), 7.08 (m, 1H), 6.45 (d, J=1.6 Hz, 1H), 6.29 (d, J=1.6 Hz, 1H), 5.29 (br s, 1H), 2.53-2.47 (m, 2H), 2.39 (s, 3H), 1.64-1.57 (m, 8H), 1.38-1.25 (m, 4H), 0.90 (t, J=6.9 Hz, 3H). ¹³C NMR (75 MHz, CDCl₃) δ 154.7, 153.1, 144.7, 137.0, 137.0, 127.7, 127.6, 126.5, 122.7, 110.9, 110.0, 108.8, 77.5, 35.7, 31.6, 30.6, 27.2, 27.2, 22.7, 21.7, 14.2. HR-MS m/z [M+H]⁺ 311.2009 (calculated for C₂₁H₂₇O₂⁺, 311.2006, 0.96 ppm error). The purity of CBN was over 95% as determined by quantitative NMR analysis.

CBN inhibited oxytosis/ferroptosis independently of cannabinoid receptors

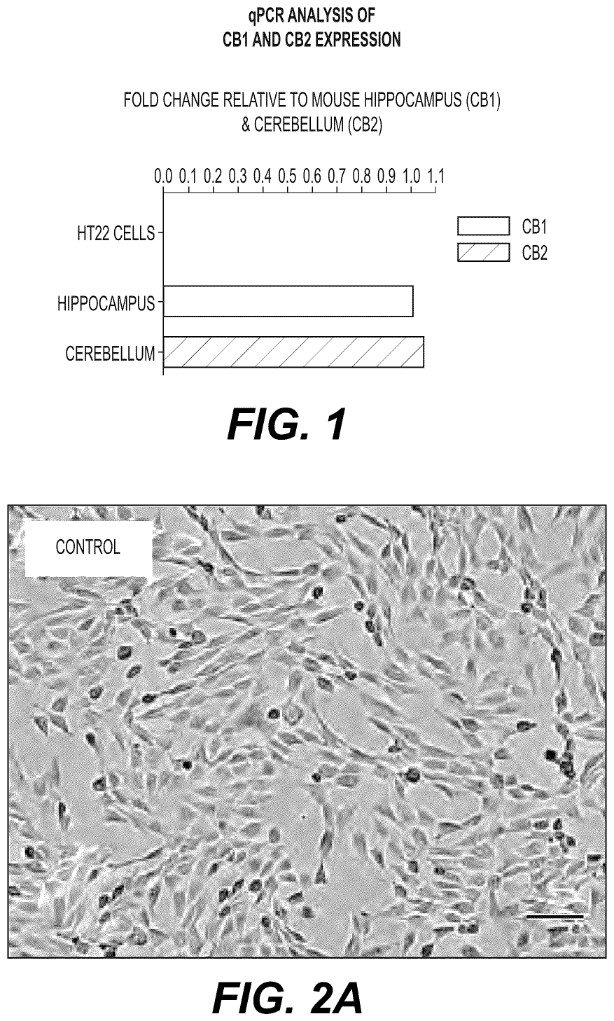

Mouse hippocampal HT22 cells were a neuronal cell line that lack cannabinoid receptors (CB1 and CB2, canonical targets of cannabinoids), as verified by qPCR analysis (FIG. 1) and prior studies. Because CBN was previously identified as a potent protector against multiple insults in HT22 cells, this cell line was used to investigate the neuroprotective mechanisms of CBN independently of cannabinoid receptor signaling.

Oxytosis/ferroptosis has been induced by inhibiting the cysteine/glutamate antiporter (system Xc-) with glutamate, leading to loss of the endogenous antioxidant glutathione (GSH), excessive calcium influx into mitochondria, production of reactive oxygen species (ROS) from mitochondria, increases in lipid peroxidation across cellular membranes, and ultimately cell death. Oxytosis/ferroptosis has been triggered by inhibition of glutathione peroxidase 4 (GPX4), a GSH-dependent antioxidant enzyme, with RSL3. System Xc- and GPX4 were upstream and downstream targets, respectively, in oxytosis/ferroptosis and they play critical roles in this regulated cell death pathway. Thus, the neuroprotection was first assessed by CBN against both glutamate and RSL3 induced cell death in HT22 cells.

Microscopic imaging clearly showed that treatment with CBN at 5 μM for 16 hr did not induce visual changes in cellular morphology compared to the control HT22 cells (FIG. 2A-B). However, treatment with a subtoxic dose of glutamate (5 mM) (FIG. 2C) or RSL3 (50 nM) (FIG. 2D) for 16 hr led to dramatic changes in cellular morphology with large numbers of rounded, shrinking, and detached cells present as compared to the control group (approximately 40-60% cell death). Such changes in morphology were characteristic of oxytosis/ferroptosis and were consistent with previous findings. Pretreatment with 5 μM CBN for 1 hr followed by co-incubation with either glutamate or RSL3 for 16 hr effectively prevented these morphological changes, as nearly all the HT22 cells were healthy, well adhered, and had fine projections similar to the control cells (FIG. 2E-F).

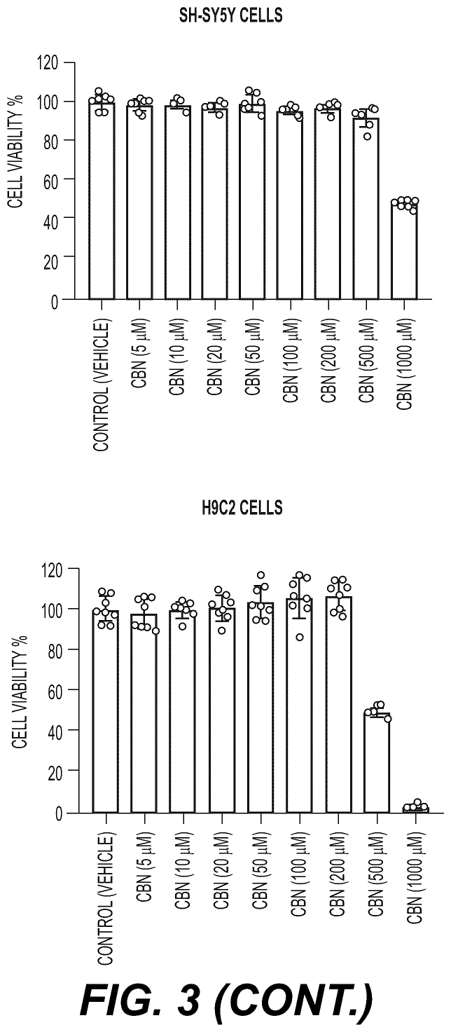

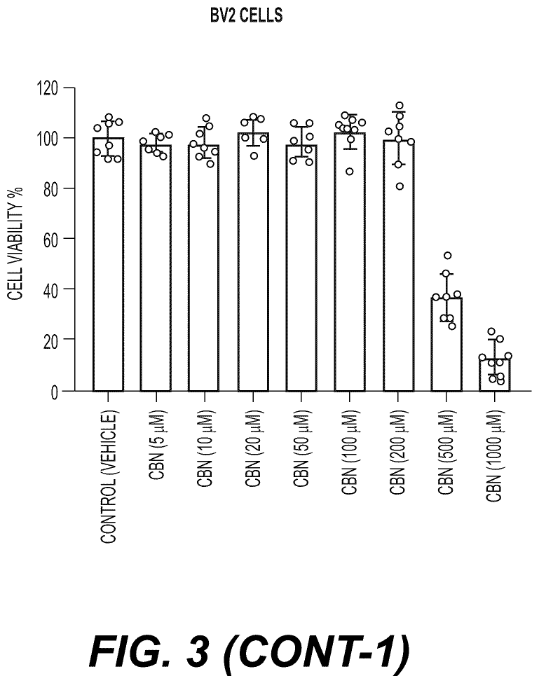

Quantitative dose-response measurements of cell viability with the 3−(4,5-dimethylthiazol-2-yl)-2,5-diphenyltetrazolium bromide (MTT) assay also showed that CBN effectively blocked the oxytosis/ferroptosis pathway induced by 5 mM glutamate (FIG. 2G) or 50 nM RSL3 (FIG. 2H) with the half maximal effective concentration (EC₅₀) values of 1.89 μM and 0.69 μM, respectively. Remarkably, no apparent cytotoxicity by CBN was observed in HT22 cells up to a dose of 200 μM (FIG. 2I), although cytotoxicity was noted at concentrations of 500 μM and above. Similar low toxicity profiles for CBN were seen in other cell lines such as PC12 pheochromocytoma cells, SH-SY5Y neuroblastoma cells, BV2 microglia, HepG2 liver cancer cells, and H₉C₂ cardiomyocytes (FIG. 3).

Together, these data suggest that CBN is a potential drug lead worthy of further investigation. Therefore, the mechanism of action of CBN against oxytosis/ferroptosis was investigated focusing on oxidative stress and mitochondrial dysfunction.

CBN suppressed mitochondrial oxidative stress in oxytosis/ferroptosis.

Activation of the oxytosis/ferroptosis pathway causes an elevation of oxidative stress, particularly in mitochondria, where mitochondrial ROS (mtROS) contribute to the lipid peroxidation of membranes. It was investigated whether CBN was capable of inhibiting the cascade of mitochondrial oxidative stress induced by oxytosis/ferroptosis.

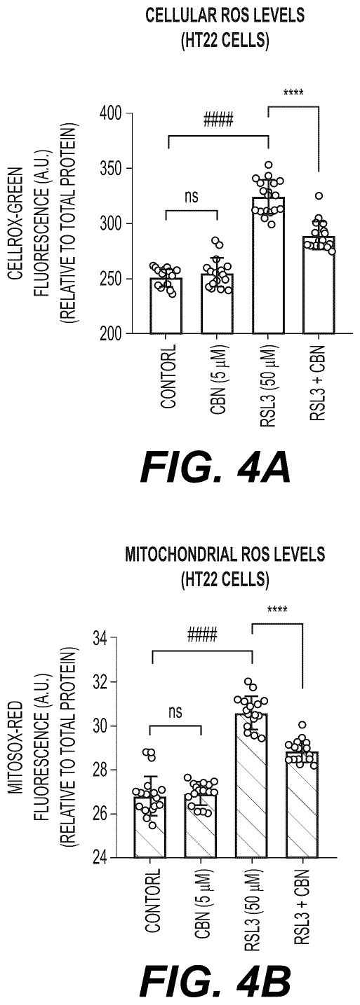

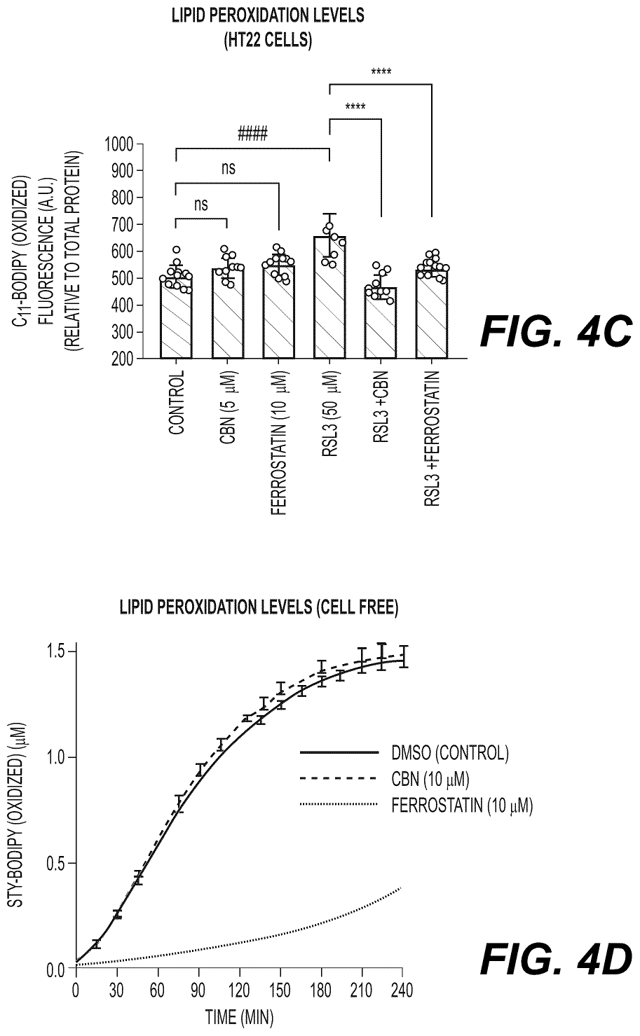

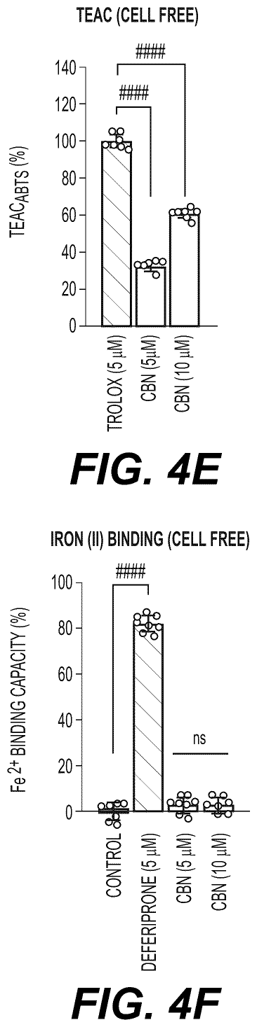

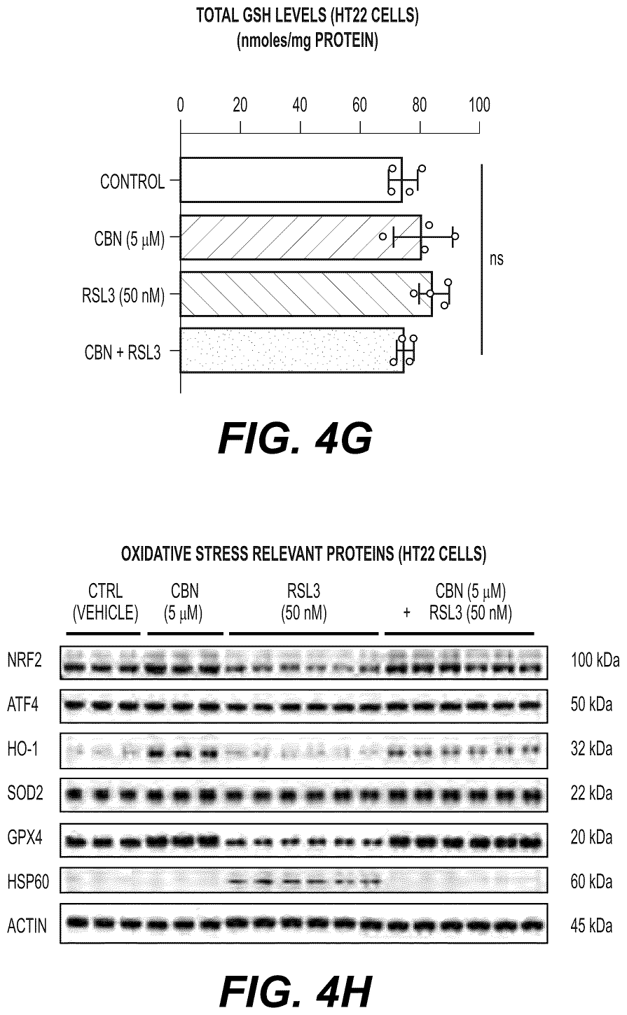

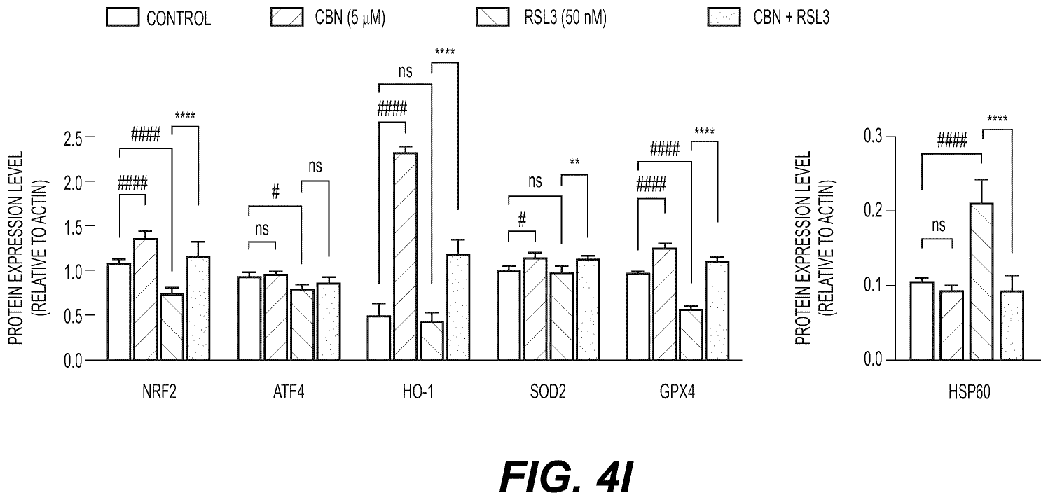

The inhibitory effect of CBN on ROS production/accumulation was investigated using two different fluorogenic probes, CellROX and MitoSOX, in live HT22 cells. The CellROX reagent is universal for cellular ROS detection, whereas the MitoSOX reagent is a ROS superoxide indicator specifically targeting mitochondria in live cells. As shown in FIG. 4A-4B, CBN (5 μM) alone did not affect the redox status of cells or mitochondria following treatment for 16 hr. However, HT22 cells treated with 50 nM RSL3 for 16 hr showed a significant increase in ROS markers in cells and in mitochondria in comparison to the control cells (p<0.0001). Treatment with CBN suppressed the RSL3-induced ROS production at both the cellular and mitochondrial levels.

Because lipid peroxidation and GSH dysregulation are major characteristics of oxytosis/ferroptosis that play key roles in the cell death pathway, it was investigated whether CBN modulated either of these two events. Ferrostatin, a known oxytotic/ferroptotic inhibitor that prevents lipid autoxidation by trapping peroxyl radicals, was used as a reference control. After treatment for 16 hr, neither CBN (5 μM) nor ferrostatin (10 μM) alone changed basal levels of lipid peroxidation in HT22 cells, whereas RSL3 (50 nM) significantly enhanced the cellular lipid peroxidation level by approximately 30% (p<0.0001) (FIG. 4C). Treatment with CBN or ferrostatin effectively suppressed the RSL3-induced cellular lipid peroxidation (p<0.0001). Surprisingly, in a cell-free, liposome-based assay of lipid peroxidation (FIG. 4D) CBN at 10 UM (a concentration 2-fold higher than in the cell-based assay) showed no inhibition relative to the control indicating that it was not a direct inhibitor of lipid peroxidation. Conversely, ferrostatin at 10 μM showed a potent effect against lipid peroxidation in the cell-free assay. The lack of anti-oxidant effects of CBN in a cell-free system was supported by the results of the TEAC assay that measured the total antioxidant capacity of compounds. The TEAC data (FIG. 4E) indicated a relatively weak capacity of CBN (5 to 10 μM) to scavenge total free radicals as compared to the standard antioxidant Trolox at 5 μM. Moreover, the possibility that CBN acts as an iron chelator in preventing iron-dependent lipid peroxidation was ruled out, as CBN at 5 to 10 μM (FIG. 4F) did not show significant Fe²⁺ binding as compared to the known iron chelator deferiprone (5 μM), which also inhibits oxytosis/ferroptosis. In addition, it was found that CBN at 5 μM had no impact on the total GSH levels in HT22 cells (FIG. 4G) regardless of its strong oxytosis/ferroptosis inhibition at the same concentration.

To further explore the antioxidant effect of CBN, the levels of several proteins known to be involved in endogenous antioxidant defenses, including nuclear erythroid 2-related factor (Nrf2), activating transcription factor 4 (ATF4), heme oxygenase-1 (HO-1), superoxide dismutase 2 (SOD2), and GPX4, were examined. Western blotting data (FIG. 4H-4I) showed that CBN alone at 5 μM was able to stimulate the upregulation of Nrf2, HO-1, SOD2, and GPX4 in HT22 cells. By contrast, RSL3 significantly decreased the expression of Nrf2, ATF4, and GPX4 in HT22 cells, and the effects were particularly substantial for Nrf2 and GPX4 (p<0.0001). Interestingly, CBN at 5 μM counteracted the effects of RSL3 and maintained all four antioxidant proteins (i.e., Nrf2, HO-1, SOD2, and GPX4) at levels similar to the control cells, whereas the effect of CBN on ATF4 was not significant. Mitochondrial heat shock protein 60 (HSP60) is a chaperone that is upregulated in response to mitochondrial stress. HSP60 was found significantly upregulated by RSL3, but this increase was suppressed by CBN (FIG. 4I). Overall, CBN was found to have a strong antioxidant potential to reduce/neutralize mtROS and lipid peroxides possibly mediated by the activation of cellular antioxidant defenses. Based on these results, CBN may have direct effects on mitochondria and thus focused our further studies on this important organelle.

CBN maintains mitochondrial calcium homeostasis in oxytosis/ferroptosis.

Mitochondrial Ca²⁺ homeostasis is essential to many neuronal functions. Excessive mitochondrial Ca²⁺ influx is detrimental to the neuron, enhances mtROS production and has been shown to take place during oxytosis/ferroptosis. Therefore, the impact of CBN on mitochondrial Ca²⁺ homeostasis during oxytosis/ferroptosis was examined.

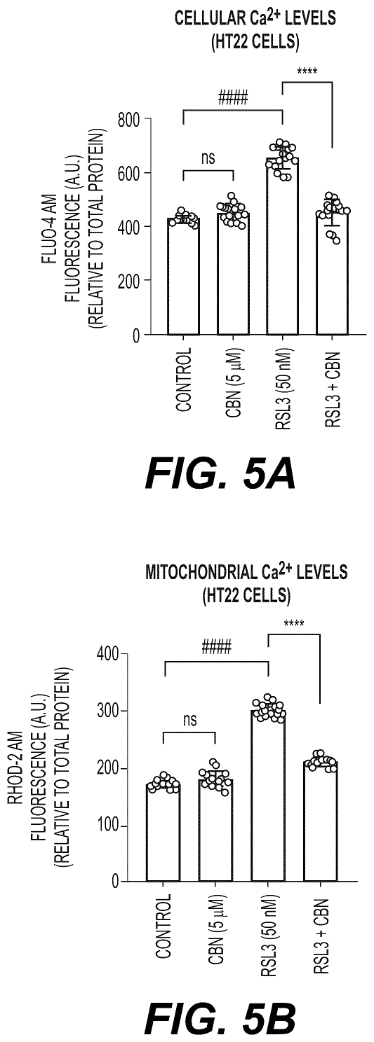

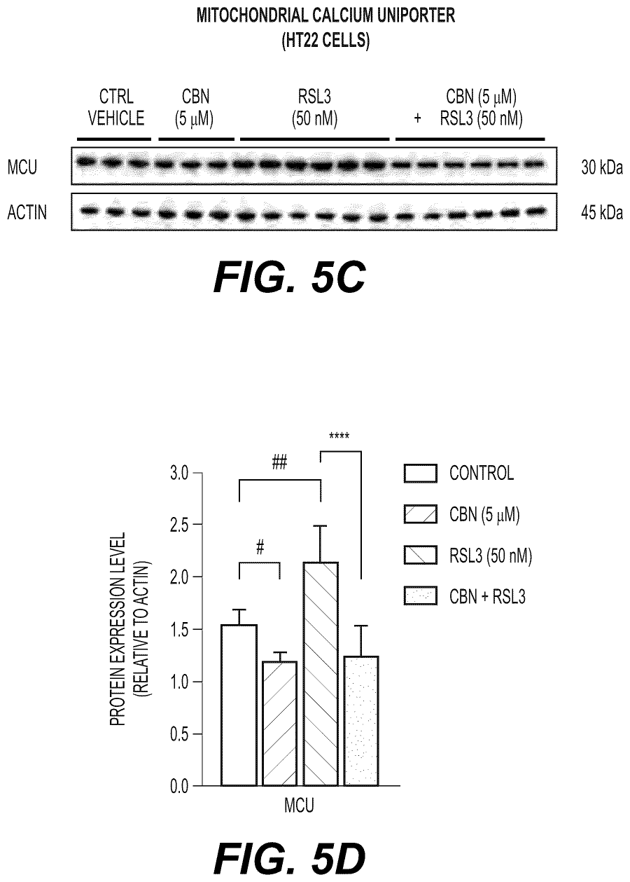

As shown in FIGS. 5A-5B, incubation of CBN at 5 UM for 16 hr did not affect Ca²⁺ influx in either the cytosol or mitochondria as determined by the calcium indicators Fluo-4 AM and Rhod-2 AM, respectively. On the other hand, RSL3 treatment for 16 hr caused a significant increase in Ca²⁺ in both the cytosol and mitochondria (p<0.0001). Pretreatment with CBN for 1 hr followed by co-incubation with RSL3 maintained basal Ca²⁺ levels in the cytosol and in mitochondria.

The mitochondrial calcium uniporter (MCU) is a key calcium channel located in the mitochondrial inner membrane that mediates the uptake of Ca²⁺ ions into the mitochondrial matrix. MCU dysregulation and malfunction have been implicated in mitochondrial bioenergetic impairment and to a greater extent in neurodegenerative disorders. Therefore, the MCU levels were examined upon compound treatment by Western blotting. Interestingly, the data (FIGS. 5C-5D) showed that treatment with CBN alone at 5 μM for 16 hr reduced basal MCU expression in HT22 cells while RSL3 upregulated MCU in the cells (p<0.0001), and CBN prevented this increase (p<0.0001). Together, the combined data from the calcium assays and the MCU measurement indicated that CBN prevented Ca²⁺ overload in mitochondria.

CBN modulates the oxidative phosphorylation (OXPHOS) system and restores mitochondrial bioenergetics.

Mitochondrial bioenergetics are involved in biochemical and molecular pathways of energy production and transformation in cells. Bioenergetics and metabolic regulation are the primary functions of mitochondria, and mitochondrial respiration in the electron transport chain (ETC) produces ATP via the OXPHOS process to fuel a variety of vital cellular functions. Disruption of mitochondrial bioenergetics can thus promote cell damage and death.

To assess mitochondrial bioenergetics, cellular respiration profiling was conducted using the Seahorse mitochondrial stress test, where cells were sequentially treated with OXPHOS inhibitors (i.e., oligomycin, carbonyl cyanide-4-(trifluoromethoxy)phenylhydrazone (FCCP), rotenone, and antimycin A). To determine whether cannabinoid receptors influence mitochondrial respiration, both HT22 nerve cells (cannabinoid receptor-deficient) and embryonic cortical neurons (cannabinoid receptor-expressing) were assayed. Based on a nearly 100% neuroprotection of CBN against 50 nM RSL3 in both HT22 cells and primary neurons (data not shown), CBN was used at 10 μM in this experiment.

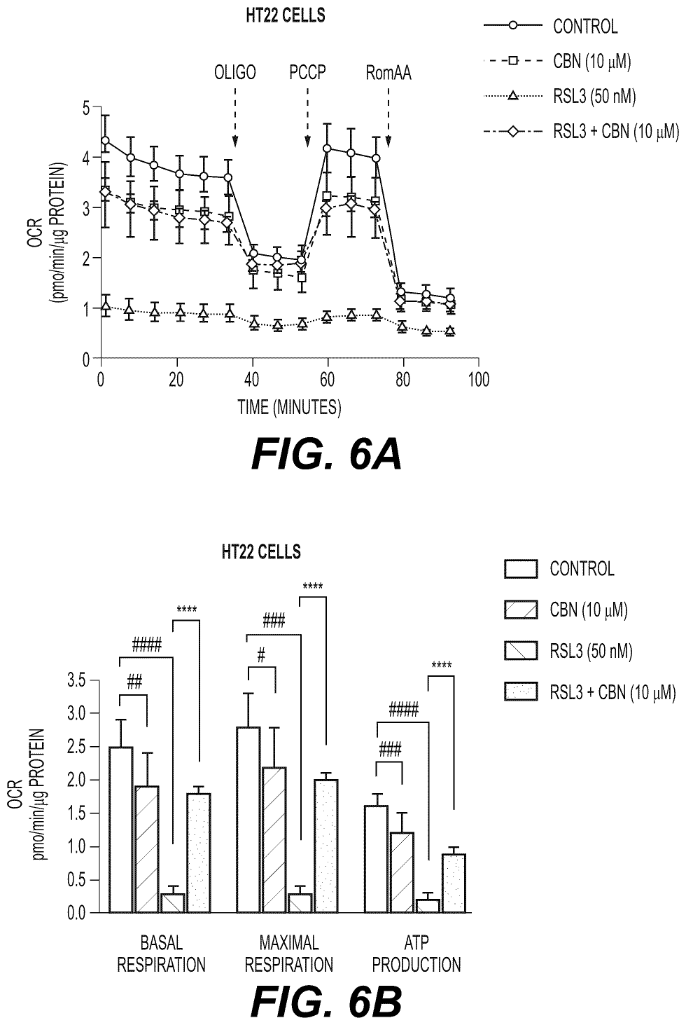

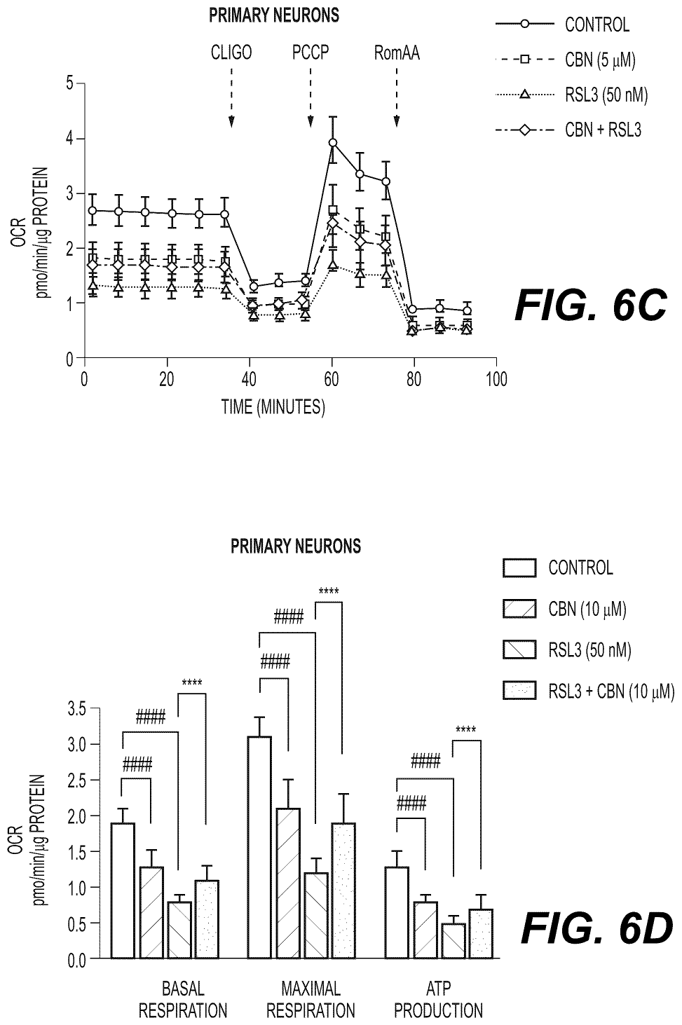

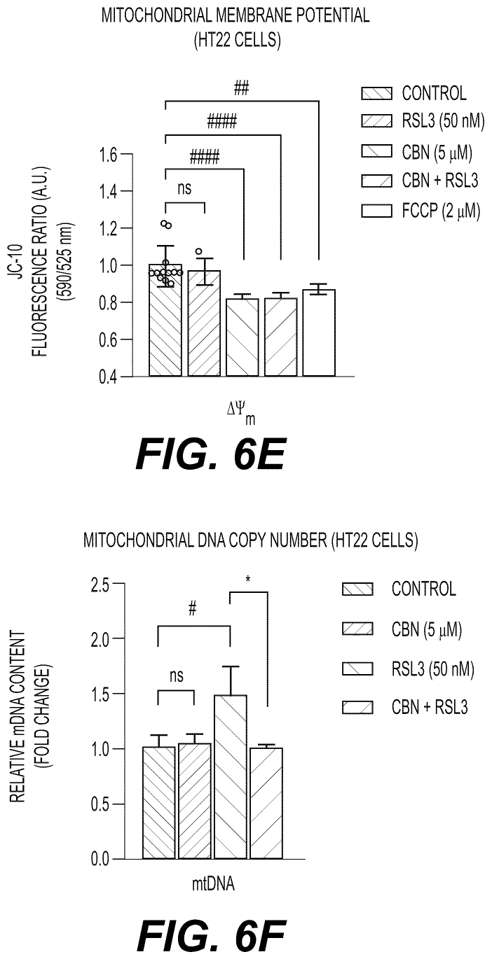

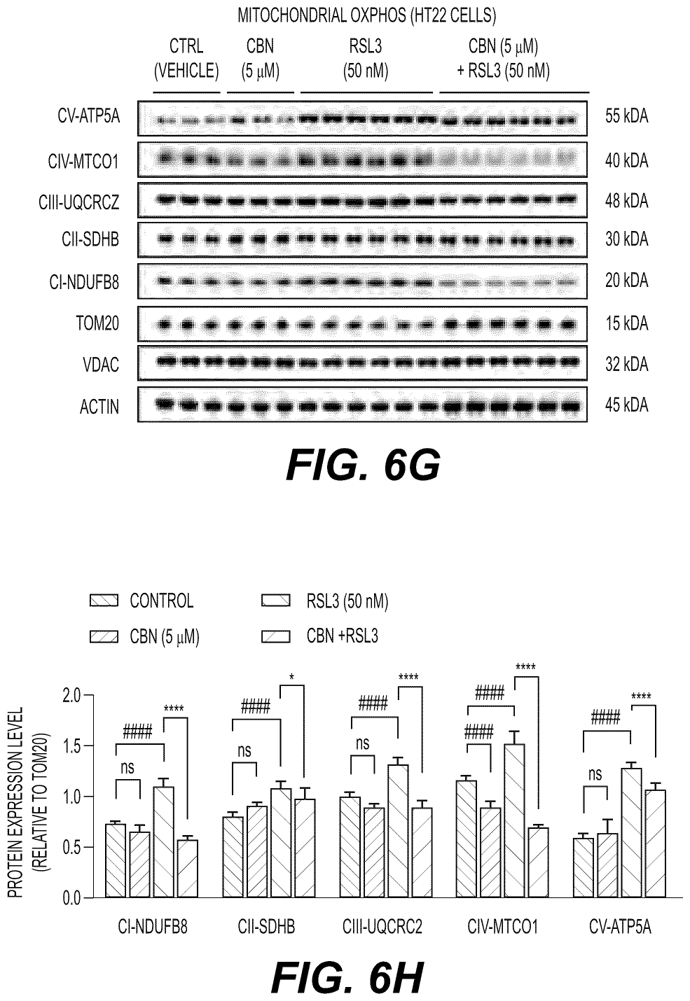

The results in HT22 cells (FIGS. 6A-6B) showed that CBN alone partially decreased both basal and maximal mitochondrial respiration, and also partially reduced ATP production. RSL3 at 50 nM almost completely depressed mitochondrial respiration as the oxygen consumption rate (OCR) barely responded to the OXPHOS inhibitors. However, HT22 cells pretreated with 10 μM of CBN followed by RSL3 treatment for 16 hr showed a maintenance of mitochondrial respiration and ATP production. Similar effects of both CBN and RSL3 on OCR profiles and mitochondrial respiration were observed in embryonic cortical neurons (FIGS. 6C-6D) in which cannabinoid receptors were highly expressed.

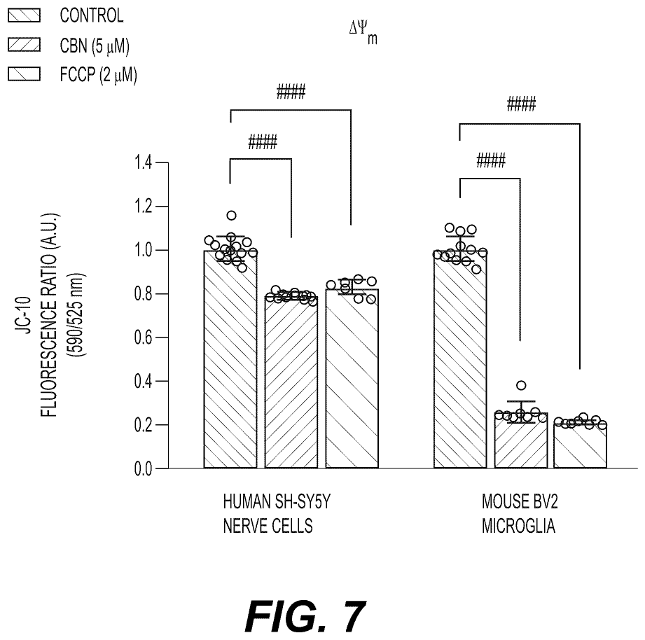

The mitochondrial membrane potential (ΔΨm) is generated by the OXPHOS proton pumps (complexes I, III and IV) across the mitochondrial inner membrane, thereby powering ATP production. It was tested whether RSL3 or CBN directly affect ΔΨm in HT22 cells. FCCP was used as a reference control because it is an uncoupling agent that decreases the proton gradient of mitochondrial OXPHOS and disrupts ΔΨm in cells. Interestingly, the data (FIG. 6E) showed that short-term treatment with RSL3 at 50 nM for 4 hr had no impact on ΔΨm. However, treatment with CBN at 5 μM for 4 hr partially decreased ΔΨm by approximately 17% in HT22 cells (p<0.0001), an effect comparable to that of FCCP (2 μM). Co-treatment with CBN and RSL3 showed an effect on ΔΨm depolarization similar to that of CBN alone. The effects of CBN on decreasing ΔΨm were also observed in SH-SY5Y neuroblastoma cells and BV2 microglia (FIG. 7).

Given these results, the effect of CBN on the mitochondrial OXPHOS protein complexes was examined. Because mtDNA was known to encode 13 proteins within complexes I, III, IV and V in the mitochondrion, all of which are involved in the OXPHOS process, the response of mtDNA copy number to the compound treatments in the cells could offer meaningful information to help elucidate how CBN affects mitochondrial bioenergetics. As shown in FIG. 6F, CBN (5 μM) alone did not induce significant changes in mtDNA in HT22 cells. However, treatment with RSL3 at 50 nM for 16 hr significantly increased mtDNA copy number compared with the control cells, and CBN was able to repress the increase in mtDNA by RSL3 and sustain mtDNA at the control level. Consistent with the mtDNA data, Western blotting (FIG. 6G-H) also showed that CBN did not exert obvious effects on the protein expression of the OXPHOS complexes I, II, III, and V, but reduced the protein level of complex IV (p<0.0001). In contrast, treatment with RSL3 at 50 nM significantly upregulated all five of the OXPHOS complexes in HT22 cells (p<0.0001). The aberrant upregulation of the OXPHOS complexes by RSL3 was inhibited by CBN with highly significant effects on the levels of complexes I, III, IV, and V (p<0.0001). Collectively, the data showed that the restoration of mitochondrial bioenergetics by CBN appears to be mediated in part by the modulation of AY′m and the OXPHOS complexes.

CBN promotes mitochondrial biogenesis.

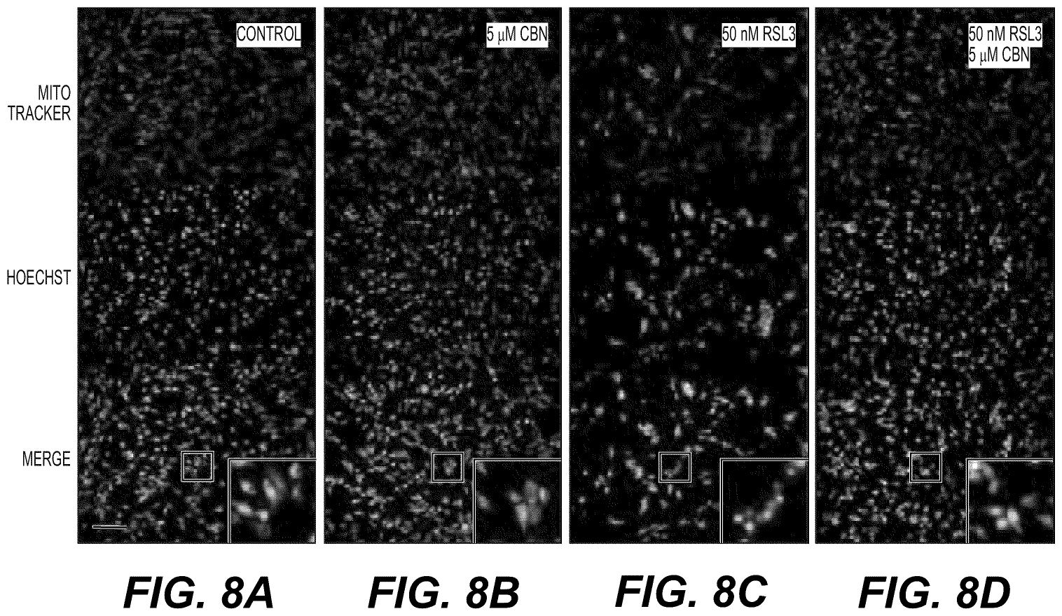

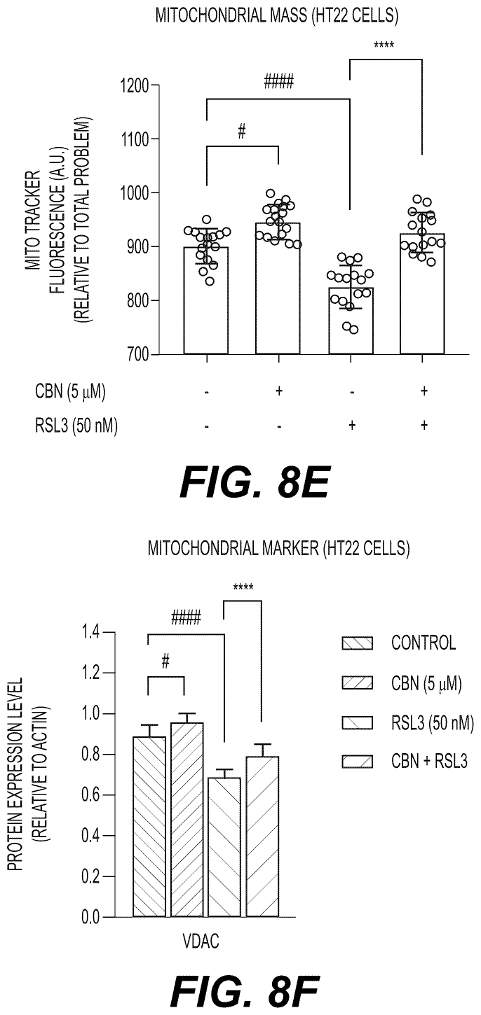

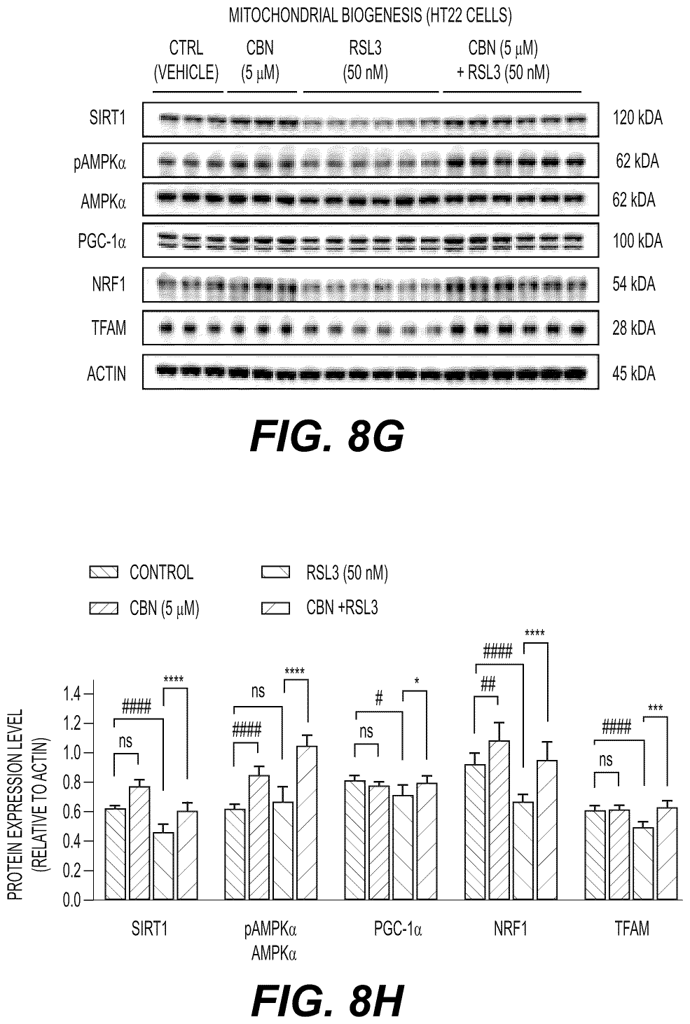

Because CBN was able to regulate mitochondrial ROS and Ca²⁺ levels and maintain mitochondrial bioenergetics, it was investigated whether these effects of CBN were correlated to mitochondrial biogenesis. To assess the overall mitochondrial content in the HT22 cells, the MitoTracker assay was used. MitoTracker fluorescent probes are cell-permeant mitochondrion-selective dyes that contain a chloromethyl moiety that covalently reacts with thiols on proteins/peptides in actively respiring mitochondria. Thus, the relative fluorescence intensity of MitoTracker in the treated cells was proportional to their corresponding mitochondrial mass in a given condition. Live-cell imaging of HT22 cells (FIGS. 8A-8B) showed that the MitoTracker dye accumulated in the CBN-treated cells with a higher intensity of fluorescence than in the control cells, while the number of cells visualized by the Hoechst nuclear staining were similar between the two groups. Quantitative analysis confirmed that 5 μM CBN treatment for 16 hr significantly increased MitoTracker fluorescence by 4.9% in HT22 cells in comparison to the control group (FIG. 8E), indicating an increase in mitochondrial mass. In contrast, treatment with 50 nM RSL3 (FIG. 8C) not only reduced the cell viability but also the mitochondrial content. As visualized by fluorescence microscopy along with quantitative measurements, the MitoTracker intensity of RSL3-treated cells was significantly decreased by 8.4% relative to that of the control cells (FIG. 8E). CBN was able to preserve the mitochondrial mass at the control level against RSL3-induced oxytosis/ferroptosis (FIGS. 8D-8E). To further confirm this observation, the expression level of the voltage dependent anion channel (VDAC) that was commonly used as a mitochondrial marker was measured. Western blotting showed that VDAC was upregulated upon CBN treatment but substantially downregulated by RSL3 (p<0.0001), and that this decrease was prevented by CBN (p<0.0001) (FIG. 8F). These results suggested that CBN and RSL3 might alter mitochondrial biogenesis.

Mitochondrial biogenesis is tightly regulated by AMP-activated protein kinase (AMPK), sirtuin-1 (SIRT1), and peroxisome proliferator-activated receptor-γ coactivator 1a (PGC-1α). Hence, the AMPK/SIRT1/PGC-1a pathway was examined and measured the protein levels of AMPK, SIRT1, PGC-1α, as well as their downstream effectors nuclear respiratory factor 1 (NRF1) and mitochondrial transcription factor A (TFAM) in HT22 cells. Western blotting (FIGS. 8G-8H) showed that CBN upregulated the protein expression of SIRT1, pAMPK(Thr172)/AMPK, and NRF1, while there were no apparent effects on PGC-1a and TFAM levels. However, the levels of all of these proteins were downregulated significantly upon RSL3 treatment and the decrease was prevented by CBN. Thus, these immunoblotting data were in agreement with the MitoTracker assay, which demonstrated that CBN activated mitochondrial biogenesis which was decreased by RSL3 treatment.

CBN regulated mitochondrial dynamics.

Mitochondria form a dynamic network with the ability to constantly elongate (fusion) and fragment (fission) within cells. In coordination with mitochondrial bioenergetics and biogenesis, mitochondrial fusion/fission dynamics play a crucial role in mitochondrial quality control and resilience against cellular stresses. Although oxytosis/ferroptosis represents a regulated cell death pathway that involves mitochondrial oxidative stress and ROS production and has been shown to induce changes in mitochondrial morphology, it has not been mechanistically examined whether oxytosis/ferroptosis causes defects in mitochondrial dynamics and if CBN can sustain a healthy balance of mitochondrial fusion and fission.

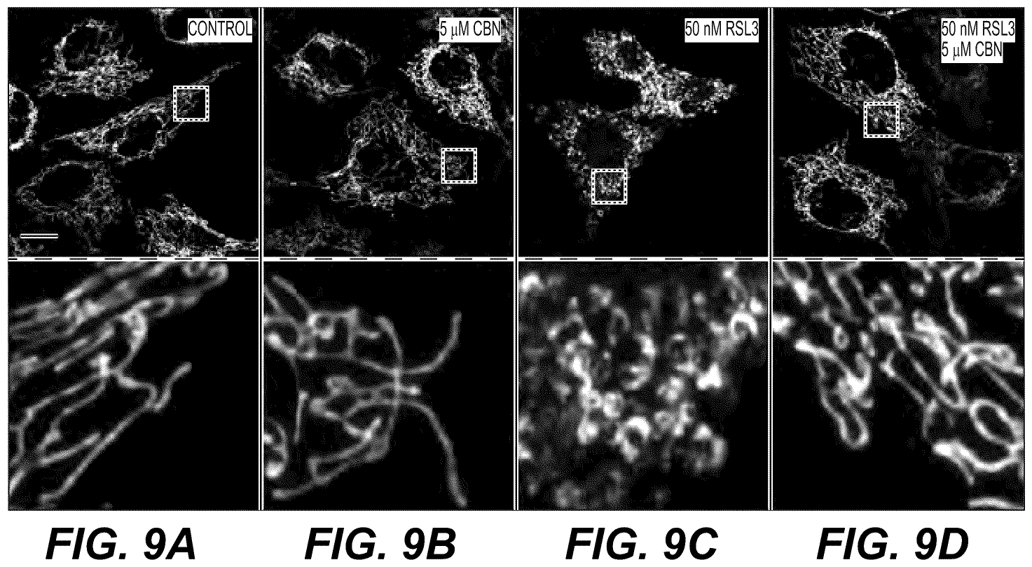

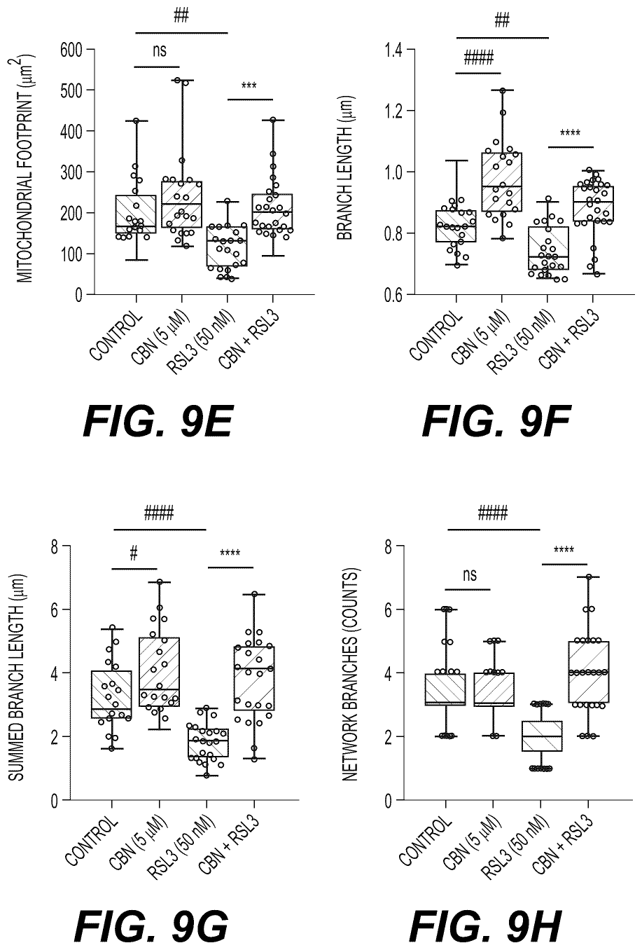

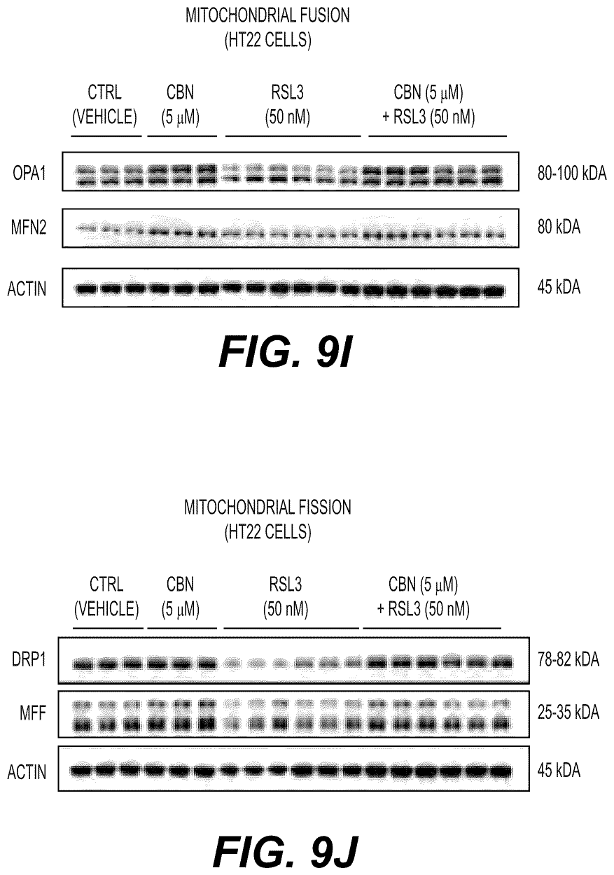

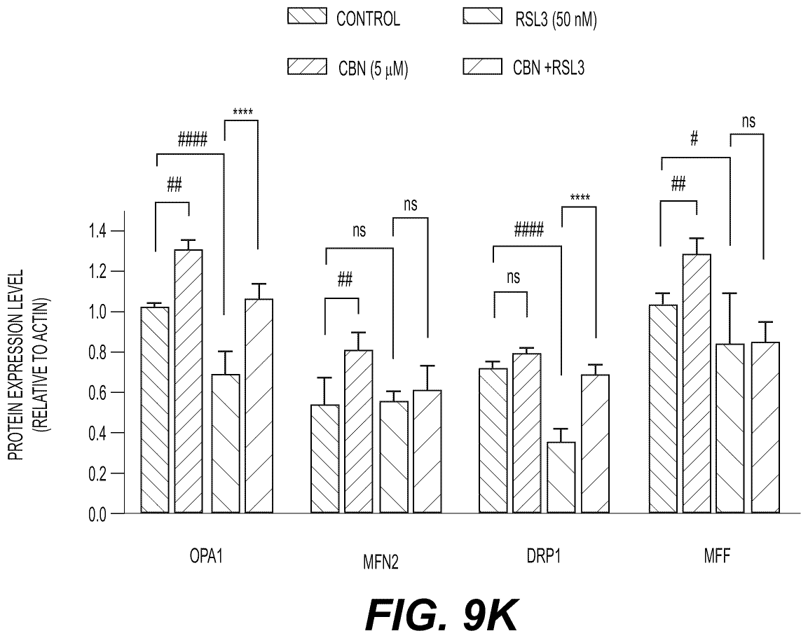

To examine the delicate changes in mitochondrial morphology in response to different treatment conditions, a HT22 mt-GFP cell line with GFP-labeled mitochondria was generated. Following the desired treatments, the cells were fixed and subjected to Airyscan super-resolution confocal microscopy analyses. As visualized in FIG. 9A, the control HT22 cells contained tubular-shaped, elongated, and branched networks of mitochondria as previously reported. The cells treated with 5 μM CBN for 16 hr (FIG. 9B) appeared to have more widespread mitochondria with highly branched networks. In contrast, the cells treated with 50 nM RSL3 for 16 hr showed a large number of fragmented, shortened and globular/donut-shaped mitochondria (FIG. 9C), similar to what has been seen in stressed, pathologic, or aging/senescent neurons. Pretreatment of CBN for 1 hr followed by RSL3 co-incubation for 16 hr was able to reduce the number of fragmented mitochondria and maintain the tubular mitochondrial morphology and networks similar to the control cells (FIG. 9D).

Quantitative image analyses of the mitochondrial morphology further supported the effects of CBN and RSL3 on mitochondrial dynamics. The mitochondrial network parameters were scored and analyzed statistically. Exposure of HT22 cells to CBN at 5 M significantly increased the individual and summed branch lengths of mitochondria (FIGS. 9F-9G), while there were no apparent effects on the mitochondrial footprints (FIG. 9E) and network branches (FIG. 9H). The results suggested an increase of mitochondrial fusion with CBN treatment. However, following exposure to 50 nM RSL3, HT22 cells showed a significant reduction in their mitochondrial footprints, mitochondrial branch lengths and network branches (FIGS. 9E-9H). This indicated a substantial fragmentation of mitochondria from large networks into small structures associated with mitochondrial fission. Treatment with CBN was able to prevent the mitochondrial fragmentation induced by RSL3 and the mitochondrial network features were improved to control levels (FIGS. 9E-9H).

The protein levels of mitochondrial fusion proteins were measured by Western Blotting including optic atrophy protein 1 (OPA1) and mitofusin 2 (MFN2), as well as mitochondrial fission proteins including dynamin-related protein 1 (DRP1) and mitochondrial fission factor (MFF) in HT22 cells. As shown in FIGS. 9I-9K, OPA1 and MFN2 were upregulated upon CBN treatment for 16 hr. In contrast, the cells treated with RSL3 significantly downregulated OPA1 while MFN2 was unaffected. Co-treatment with CBN and RSL3 enhanced the expression of OPA1 but not MFN2. Regarding the mitochondrial fission proteins, CBN significantly enhanced the expression of MFF but not DRP1, while RSL3 decreased the expression levels of both DRP1 and MFF in HT22 cells (FIGS. 9J-9K). Co-treatment with CBN and RSL3 restored DRP1 levels but not MFF levels. Overall, the super-resolution microscopy and immunoblotting data suggested that CBN was an effective modulator of mitochondrial dynamics against oxytosis/ferroptosis.

CBN required functional mitochondria to protect against oxytosis/ferroptosis.

Given the multiple effects of CBN on mitochondria, it was investigated if mitochondria play an essential role in the protective effects of CBN against oxytosis/ferroptosis. To do this, a HT22 mt-GFP/mCherry-Parkin cell line was generated, which overexpresses the ubiquitin E3 ligase Parkin protein in addition to the GFP-labeled mitochondria. As characterized previously, overexpression of Parkin coupled with treatment with mitochondrial uncouplers (e.g., FCCP) activates mitophagy and thus can be used to experimentally eliminate mitochondria partially or completely. Using the HT22 mt-GFP/mCherry-Parkin cells and mt-GFP cells as a wild type (WT) control, it was investigated whether the protective effects of CBN against oxytosis/ferroptosis persisted after mitochondrial clearance.

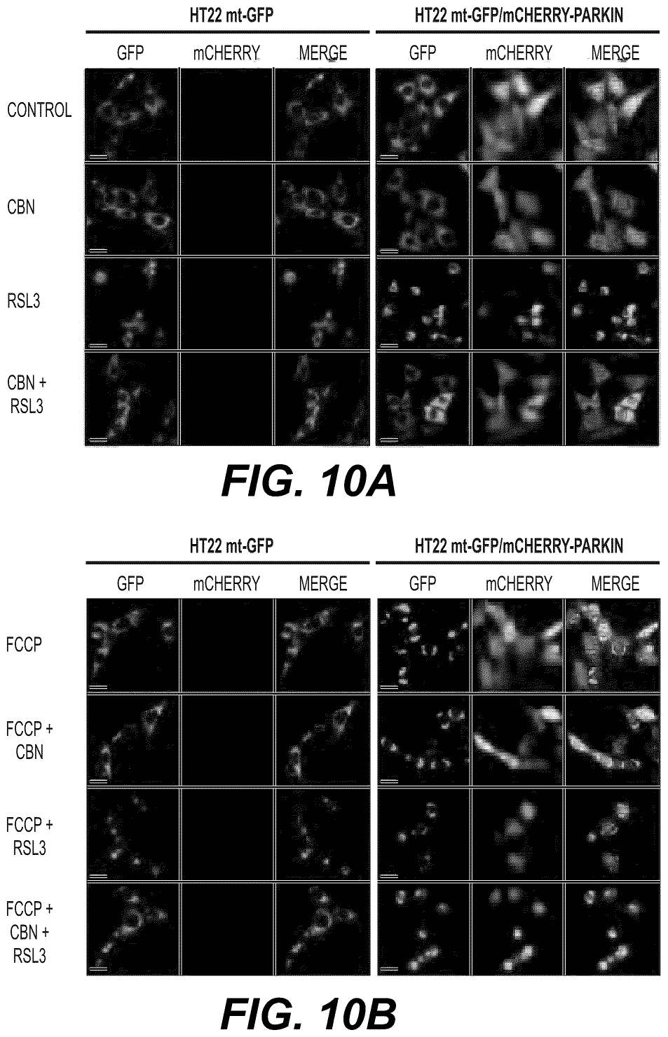

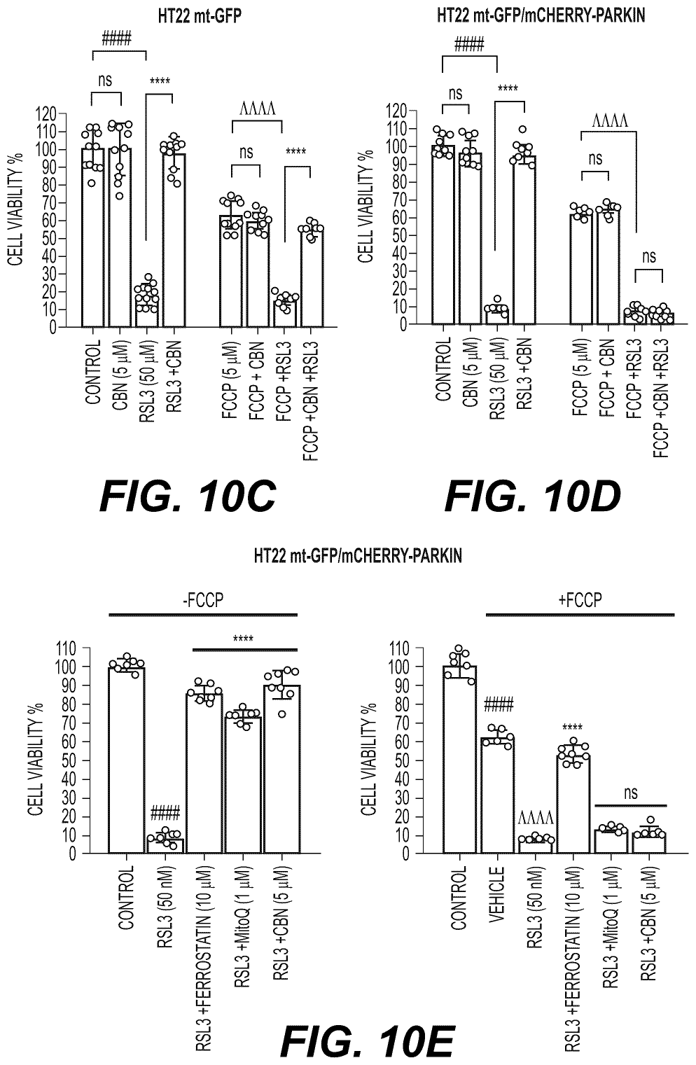

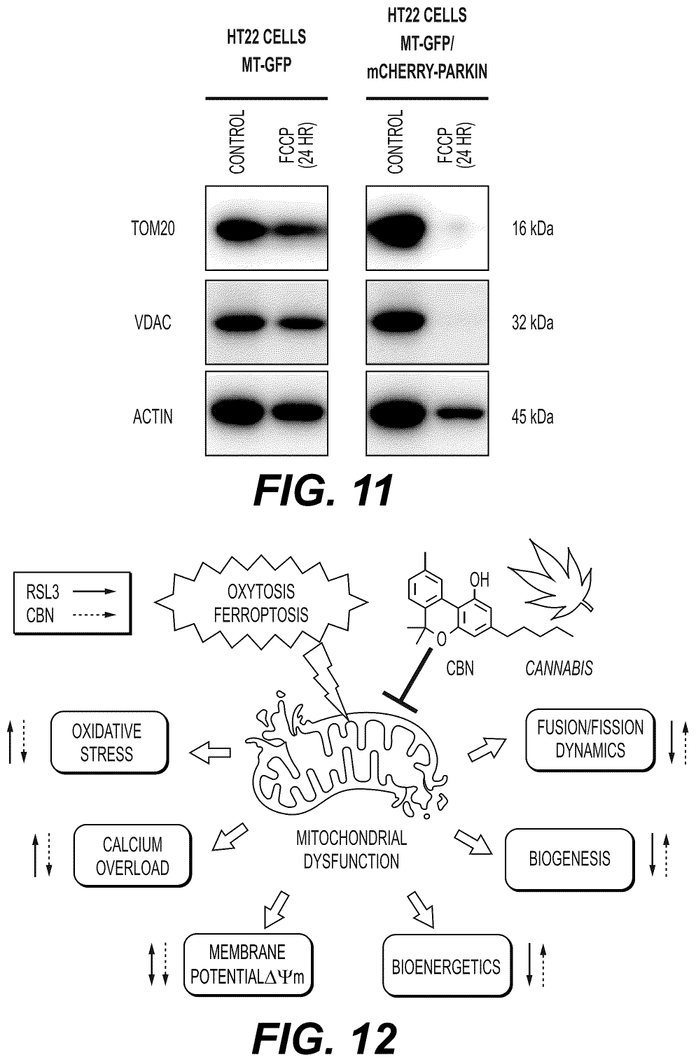

The differential cellular phenotypes were analyzed under eight treatment conditions with the mt-GFP WT or mt-GFP/mCherry-Parkin cells treated with vehicle, CBN, RSL3, CBN+RSL3, FCCP, FCCP+CBN, FCCP+RSL3, and FCCP+CBN+RSL3, respectively. To determine if CBN offered similar protection in the two cell lines in the presence of mitochondria, the mt-GFP WT or mt-GFP/mCherry-Parkin cells seeded at the same density were treated with vehicle, CBN, RSL3, or CBN+RSL3 for 16 hr. Fluorescence microscopic images (FIG. 10A) illustrate that CBN exhibited strong and equivalent protection against RSL3-induced cell death in both cell lines. When both were pretreated with FCCP (5 μM) for 24 hr (FIG. 10B), the mt-GFP/mCherry-Parkin cells showed a partial removal of mitochondria as evidenced by fewer and compressed/clustered GFP-labeled mitochondria mostly surrounding the nucleus, indicative of widespread mitophagy. Western blotting confirmed a substantial reduction in the expression of mitochondrial markers (i.e., VDAC, TOM20) in the HT22 mt-GFP/mCherry-Parkin cells after FCCP treatment for 24 hr (FIG. 11). In contrast, FCCP did not induce mitochondrial loss in the mt-GFP WT cells as the GFP-labeled mitochondria were extensively distributed in the soma and their mitochondrial markers were well retained (FIGS. 10B and 11). As shown in FIG. 10B, CBN treatment did not alter the FCCP-induced loss of mitochondria in the mt-GFP/mCherry-Parkin cells. After FCCP treatment, both cell lines still responded to RSL3 treatment becoming rounded, shrinking and undergoing the typical oxytotic/ferroptotic cell death process. Importantly, while CBN protected the mt-GFP WT cells from RSL3, it failed to protect the mt-GFP/mCherry-Parkin cells from oxytosis/ferroptosis (FIG. 10B, bottom row of panel).

In addition to fluorescence microscopy, quantitative cell viability assessments on both cell lines following the different treatment conditions were performed. To account for mitochondrial depletion, instead of using the MTT assay that depends on mitochondrial metabolism, a DNA-based cell proliferation assay was used. In concordance with the microscopic observations, CBN had similar protective effects against RSL3 in both the mt-GFP WT and mt-GFP/mCherry-Parkin cells when they were not pretreated with FCCP (FIGS. 10C-10D). Both cell lines showed a decreased cell number in response to FCCP treatment after 40 hr (about 40% decrease in cell viability compared to the vehicle control), which could be due to the uncoupling effect of FCCP on the ETC and consequent decreases in ATP production required for cell proliferation. After FCCP pretreatment for 24 hr, co-treatment with CBN of both cell cultures for an additional 16 hr did not further change the cell numbers. RSL3 induced a dramatic decrease in cell viability down to approximately 10-15% in both the FCCP pretreated mt-GFP WT and mt-GFP/mCherry-Parkin cells. In contrast, CBN treatment of RSL3-treated cells showed very different effects between the two cell lines (FIGS. 10C-10D). While CBN was protective in the mt-GFP WT cells, it did not protect in mt-GFP/mCherry-Parkin cells with partial mitochondrial clearance.

To provide further support for the critical role of mitochondria in the protective effects of CBN against oxytosis/ferroptosis, two oxytotic/ferroptotic inhibitors, ferrostatin and MitoQ with distinct biochemical mechanisms, were also evaluated and compared to CBN in the HT22 mt-GFP/mCherry-Parkin cells. Ferrostatin is a radical-trapping antioxidant that does not rely on mitochondria to inhibit oxytosis/ferroptosis, and as mentioned above it showed a potent anti-lipid autoxidation effect different from CBN in the cell-free, liposome-based assay (FIG. 10D). By contrast, MitoQ is a selective mitochondrially-targeted antioxidant. As shown in FIG. 10E, without FCCP pretreatment the three compounds effectively protected from RSL3-induced cell death. However, in the FCCP-mediated mitochondria-depleted cells, only ferrostatin rescued the cells from RSL3 treatment, whereas MitoQ showed no cytoprotection, similarly to CBN. Together, these results established that the protective effects of CBN against oxytosis/ferroptosis physically require functional mitochondria.

CBN is a minor phytocannabinoid derived from oxidative degradation of THC, mostly found in aged Cannabis. CBN is a weak agonist of CB1/CB2 receptors with 10 to 100-fold lower binding affinities compared to THC and the affinity is tissue- or cell-type specific. Thus, CBN is generally considered as a non-psychoactive phytocannabinoid and is not listed as a Schedule I controlled substance by the U.S. Drug Enforcement Administration (US-DEA) nor in the Schedules of the United Nations International Drug Control Conventions. In silico physicochemical prediction indicates that CBN has a relatively good profile of CNS drug-like properties that obeys the Lipinski's rule of five. Owing to the presence of an additional aromatic ring as compared to THC, CBN is metabolically more stable and its bioavailability after oral or inhaled administration has been reported in the range of 10 to 40%. Preclinical and limited clinical evaluations have also shown that CBN has good brain penetrance with excellent safety and pharmacokinetic profiles in animals and humans. However, pharmacological and mechanistic studies on CBN as a treatment for age-associated neurodegenerative diseases have not been reported. In the present study, CBN was demonstrated to effectively protect from both glutamate and RSL3-induced oxytosis/ferroptosis (FIG. 2) and it had a large safety margin and low cytotoxicity across different cell types.

Oxytosis/ferroptosis recapitulates several aspects of mitochondrial pathology that are relevant to neurodegenerative diseases [6, 41]. Furthermore, mitochondrial oxidative stress is a major pathological hallmark of neurodegenerative disorders including AD, PD, and HD. As shown in FIG. 4, CBN potently prevented both cellular and mitochondrial ROS production induced by oxytosis/ferroptosis. Although CBN effectively inhibited cellular lipid peroxidation, its action against cell-free lipid autoxidation was negligible. Given that lipid peroxidation is generally considered as an executive step leading to the disruption of cellular membrane integrity downstream in the oxytosis/ferroptosis pathway, the discrepancy between the cell-based and cell-free assays suggested that CBN does not directly inhibit RSL3-induced lipid peroxidation. This idea was supported by the results of the cell-free TEAC and iron binding assays that indicated that CBN, albeit its strong cytoprotection at relevant doses (5 to 10 μM), was neither an effective free radical scavenger nor an iron chelator. In addition, CBN showed no impact on the total cellular GSH levels regardless of RSL3 co-treatment. Because RSL3 directly inhibits GPX4, an enzyme that is dependent on GSH, it was likely that CBN acted on targets downstream of GSH synthesis but upstream of lipid peroxidation in the oxytosis/ferroptosis pathway.

Immunoblotting demonstrated that CBN was able to activate the antioxidant defense system via the upregulation of Nrf2, HO-1, SOD2 and GPX4 in HT22 cells. While no effect on cellular GSH synthesis was seen, CBN appears to promote a plethora of other endogenous antioxidant mediators that could reduce/neutralize ROS and lipid peroxides against oxidative injury. In parallel, a strong increase was found in the mitochondrial chaperone HSP60 by RSL3 in response to mitochondrial oxidative stress that was effectively prevented by CBN, suggesting that CBN might be specifically acting on mitochondria to prevent stress. To address this question, the effects of CBN were examined on central aspects of mitochondrial function both alone and in the presence of RSL3.

Numerous studies have shown that prolonged mitochondrial Ca²⁺ overload led to increased ROS generation, bioenergetic and metabolic disturbance, and induction of cell death associated with mitochondrial oxidative stress. RSL3 increased mitochondrial Ca²⁺ which was inhibited by CBN possibly, at least in part, via the downregulation of MCU (FIG. 4). Owing to the pathophysiological relevance of MCU in neurodegenerative diseases and its role in Ca²⁺ -dependent mitochondrial bioenergetics, the effects of CBN on Ca²⁺ regulation could be a beneficial feature for neurotherapies.

Furthermore, CBN was demonstrated to directly modulate mitochondrial bioenergetics against oxytosis/ferroptosis (FIG. 5). Consistent with recent reports on RSL3-mediated bioenergetic impairments in mouse embryonic fibroblasts (MEF) and hepatocellular carcinoma (HCC), the real-time mitochondrial metabolic analysis clearly illustrated that RSL3 caused mitochondrial dysfunction at least partly by decreasing electron flow through the ETC, resulting in a reduction in mitochondrial respiration and cellular ATP production in both cannabinoid receptor-deficient (i.e., HT22 cells) and cannabinoid receptor-expressing cells (i.e., embryonic cortical neurons). CBN maintained the bioenergetic phenotype against the damaging effects of RSL3 and showed comparable respiratory profiles in both cell types. These results supported the idea that at least part of the neuroprotective effects of CBN against oxytosis/ferroptosis could be due to the maintenance of mitochondrial bioenergetics regardless of the presence of cannabinoid receptors in the cells.

Since the generation of mtROS mainly takes place at the ETC during OXPHOS, particularly at complexes I, II and III, the upregulation of OXPHOS complexes induced by RSL3 was plausibly a compensatory response to the decrease in ETC activity, and this also correlated well with the significant accumulation of mtROS in the cells (FIG. 4). This aberrant mtROS production could then further impair the OXPHOS process for mitochondrial respiration providing a feed forward loop which promoted cell death. By contrast, CBN counteracted these effects on mitochondria. Intriguingly, short-term treatment with CBN depolarized ΔΨm in HT22 cells, whose effect was analogous to that of the mitochondrial uncoupler FCCP and was not affected by RSL3 treatment (FIG. 5E). Our data were consistent with previous reports which showed that transient or modest depolarization of mitochondrial membranes (e.g., using mitochondrial uncouplers) could diminish mtROS production and mitochondrial respiration. Thus, CBN showed a direct effect on mitochondria and might subtly modulate ΔΨm for neuroprotection through a similar mechanism. Moreover, because of its high lipophilicity (cLogP=7.4, TPSA=29) (FIG. 1), CBN could possibly diffuse or anchor onto mitochondrial membranes to alter the membrane fluidity which has been shown to affect the efficiency of mitochondrial respiration. Overall, the data support the idea that CBN can prevent the toxic effects of RSL3 through suppressing mtROS and Ca²⁺ uptake, depolarizing ΔΨm, downregulating the increase in the OXPHOS complexes, and eventually preserving mitochondrial bioenergetics in the cells.

Besides mitochondrial oxidative stress and bioenergetics, the impact of RSL3 on mitochondrial biogenesis and fusion/fission dynamics offers new insights into the effects of oxytosis/ferroptosis on mitochondrial function beyond the classical markers of redox biology. It was shown that RSL3 induces the dysregulation of mitochondrial biogenesis plausibly through interfering with the AMPK/SIRT1/PGC-1α pathway, whereas CBN conferred a beneficial effect by stimulating mitochondrial biogenesis against RSL3 (FIG. 6). Additionally, extending the phenotypic observations by Jelinek et al., this study found that mitochondrial fragmentation occurred in response to RSL3 treatment, indicating an imbalance in dynamics towards fission. CBN was able to restore this fusion/fission balance (FIG. 9). Changes in mitochondrial morphology have been implicated in the modulation of bioenergetics, OXPHOS efficiency, and quality control in mitochondria in response to cellular stresses, insults, or the aging process. Particularly, elongated mitochondria have been shown to restore mitochondrial respiration and ATP production as well as redistribute biomaterials to rescue defective mitochondria, all of which were indeed found to be modulated by CBN (FIG. 5). Because CBN promoted biogenesis to generate new and healthy mitochondria to fuel mitochondrial fusion, this could potentially stimulate the upregulation of mitochondrial fusion proteins like OPA1 and MFN2. On the other hand, CBN facilitated the fission process to remove damaged mitochondria possibly via intervening in the AMPK/MFF/DRP1 pathway. Overall, it appeared that CBN exerted its mitochondrially protective effects at least partly by promoting mitochondrial biogenesis, restoring mitochondrial fusion, and sustaining mitochondrial bioenergetics in the cells (FIG. 5-9).

Finally, the results obtained from an inducible, mitochondrially-depleted cell line (FIG. 8) along with the ΔΨm assay (FIG. 5E) reinforced the idea that CBN inhibits oxytosis/ferroptosis through directly targeting mitochondria and that its neuroprotective effects rely on functional mitochondria in the cells. These novel observations also indicate that while GPX4-dependent oxytosis/ferroptosis induced by RSL3 does not require mitochondria to initiate cell death, the maintenance of healthy mitochondria still provided cytoprotection. Therefore, these results suggested that targeting mitochondria in general against age-related insults, regardless of the direct/indirect impact of these insults on mitochondria, was a promising therapeutic strategy for age-associated neurodegenerative diseases.

In this study, key aspects of mitochondrial physiology were examined to determine the mechanism of action underlying the neuroprotective effects of CBN against oxytosis/ferroptosis. In this comprehensive investigation, the critical role of mitochondria in CBN-mediated protection was identified against the oxytosis/ferroptosis pathway by examining the interplay between oxidative stress, Ca²⁺ uptake, membrane potential, bioenergetics, biogenesis, and fusion/fission dynamics (FIG. 12). This was the first report using a mitochondrially-depleted cell line as a novel approach to demonstrate that functional mitochondria were pivotal for the protection against oxytosis/ferroptosis mediated by a small molecule. Additionally, the new findings uncovered that CBN maintains mitochondrial homeostasis against oxytosis/ferroptosis, even when the initiation of cell death appeared not to require mitochondria. It effectively reduced mitochondrial oxidative stress in terms of mtROS and lipid peroxidation and prevented mitochondrial Ca²⁺ dysregulation. These effects of CBN were at least in part involved in the activation of endogenous antioxidant defenses and the downregulation of MCU. Moreover, CBN directly targeted mitochondria to modulate ΔΨm and the OXPHOS complexes, resulting in the restoration of mitochondrial bioenergetics as well as the regulation of mitochondrial biogenesis and dynamics to improve mitochondrial health.

Given the biological importance and therapeutic relevance of mitochondrial dysfunction in many age-associated neurodegenerative diseases, an in-depth understanding of the root cause and mechanistic links between oxytosis/ferroptosis and mitochondrial pathophysiology in terms of signal transduction, metabolic pathways, and cellular phenotypes warrants further investigation. Most importantly, this study offered strong evidence that non-psychoactive phytocannabinoids such as CBN elicited neuroprotective actions independent of the canonical CB1/CB2 pathway and that they could serve as valuable CNS drug leads.

### Example 2: Cannabinol Purity

A study was conducted to determine the purity of cannabinol as provided herein and is shown in Table 3.

TABLE 3
Results of purity analysis of CBN

| | | | | % RPD (15% |
|---|---|---|---|---|
| | Averaged | Primary | Duplicate | Action Level) |
| Total THC | <LOQ(0.1577%) | <LOQ % | <LOQ % | 0% PASS |
| Total CBD | <LOQ(0.0431%) | <LOQ % | <LOQ % | 0% PASS |
| CBN | 98.9% | 98.16% | 99.64% | |
| Pesticides | PASS | PASS | PASS | |
| Solvents | PASS | PASS | PASS | |

LOQ: level of quantification

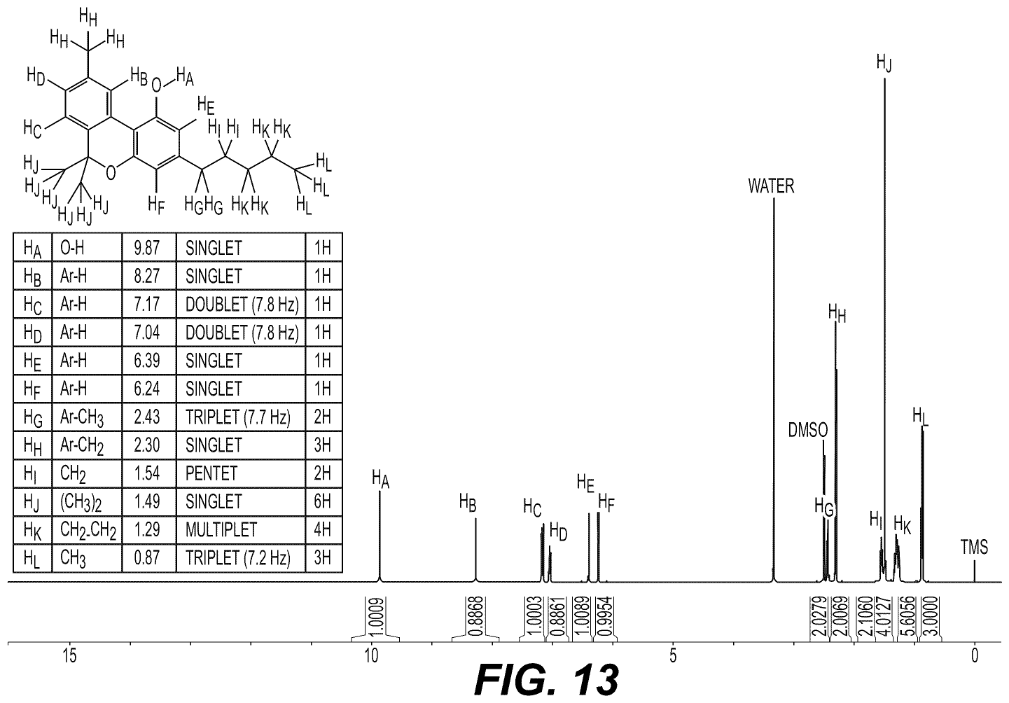

An NMR analysis of the cannabinol as provided herein is shown in FIG. 13.

### Example 3: Cannabinol (CBN), Dihydrocannabinodiol (H₂CNBD), and Acetylcannabinol (CBN—OAc) Gas Chromatography-Mass Spectrometry (GCMS) Study

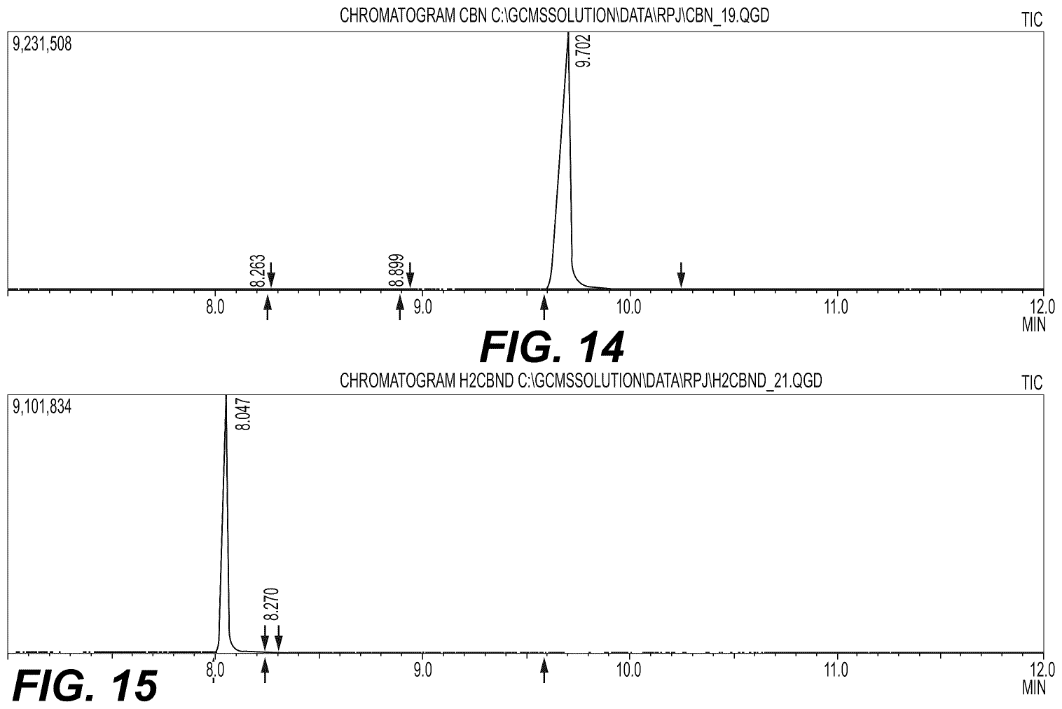

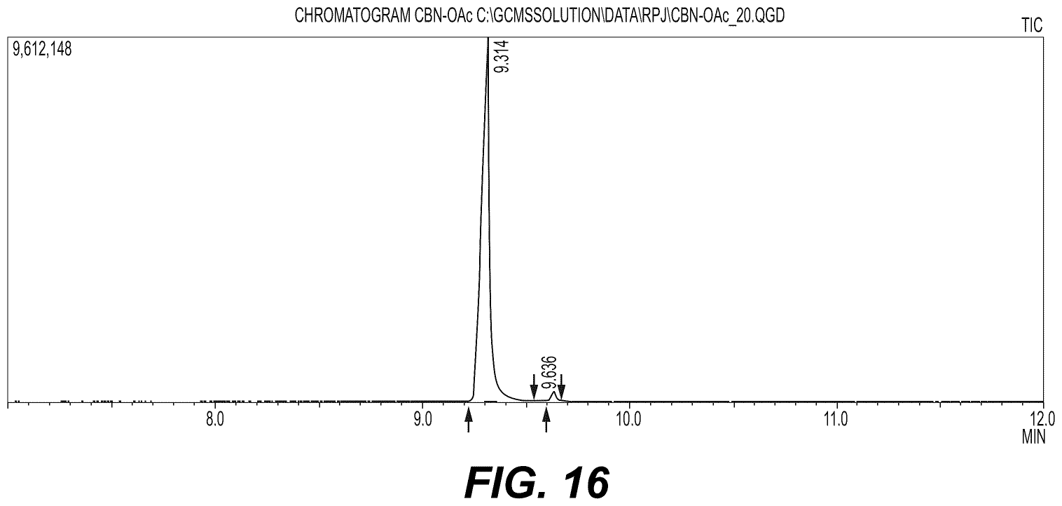

GCMS of CBN, H₂CNBD, and CBN—OAc were taken and are shown in FIGS. 14-16.

### Example 4: Cannabinol Mouse Study

### Materials and Methods

Study Design: A group of 9-months-old female senescence-accelerated prone 8 (SAMP8) mice are fed with vehicle diet (LabDiet 5015, TestDiet, Richmond, IN), and a second and third group of 9-months-old female SAMP8 mice are fed with cannabinol diets (LabDiet 5015+400 ppm cannabinol, LabDiet 5015+200 ppm cannabinol, TestDiet, respectively). Diet treatment lasts for four months until mice reached 13 months of age. At 9 months of age, SAMP8 mice already present a strong phenotype. A fourth group of 9-months-old female SAMP8 mice are used as the baseline control group.

The effect of the cannabinol is assessed in older SAMP8 mice after the four months of treatment and any age-related changes are defined by comparison to 9 months old SAMP8 mice. All mice are randomly assigned to experimental groups. The number of mice per group is determined based on sufficiency to attain statistical power. Behavioral testing is carried out one month prior to sacrifice and collection of biological material. Data is analyzed by blinded researchers when appropriate.

SAMP8 Mice. The SAMP8 line, a naturally occurring mouse line that was developed based on its phenotype of accelerated aging, is acquired from Harlan Laboratories (U.K.). Mouse body weights are measured regularly. All experiments are performed in accordance with the US Public Health Service Guide for Care and Use of Laboratory Animals.

Tissue Preparation: Mice are anesthetized and their blood collected by cardiac puncture. After perfusing with PBS, their brains are removed and dissected to collect cortex and hippocampus. Tissue are prepared for Western blotting, RNA extraction and metabolomic analysis.

Measurement of acetyl-CoA. Acetyl-CoA levels are determined in protein-free lysates of primary neurons or HT22 cells treated as indicated in the figure legends using a kit from Sigma (MAK039) according to the manufacturer's instructions. The levels are normalized to the protein in the solubilized pellet using the BCA assay.

Cell Lines. Mouse hippocampal HT22 cells are propagated as previously described (Davis J B, Maher P. 1994. Protein kinase C activation inhibits glutamate-induced cytotoxicity in a neuronal cell line. Brain Research 652:169-173). To prevent cell misidentification, large batches of each cell line are frozen that are regularly thawed to avoid using the wrong cell line. Cell lines are routinely tested for mycoplasma.

Primary Neurons. Primary cortical neurons are prepared from day 17 rat embryos and used at 7 days in vitro (7 DIV).

Behavioral Assay. Elevated plus maze: The maze consists of four arms (two open without walls and two enclosed by 15.25 cm high walls) 30 cm long and 5 cm wide in the shape of a plus. A video-tracking system (Noldus Etho Vision) is used to automatically collect behavioral data. The software is installed on a PC computer with a digital video camera mounted overhead on the ceiling, which automatically detects and records when mice enter the open or closed arms of the maze and the time spent in each. Mice are habituated to the room 24 hr before testing and are habituated to the maze for 1 min before testing by placing them in the center of the maze and blocking entry to the arms. Mice are then tested for a 5 min period and their behavior recorded. Disinhibition is measured by comparing the time spent on the open arms to time spent on the closed arms. Barnes maze: The maze consists of a flat circular surface (36′ diameter) with 20 equally spaced holes (2′ diameter) along the outer edge. One of the holes leads to a dark hide box while the other 19 led to false boxes that are too small to be entered. The latency to enter the hide box is recorded. The test is conducted in three phases. Phase 1 (Training): A hide box is placed under one of the holes. Animals are placed into an opaque cylinder in the center of the maze for 30 seconds to promote spatial disorientation at the start of the test. After 30 seconds, the cylinder is removed and the animal explores the maze until it finds and enters the hide box. The number of incorrect entries is scored. If the mouse fails to enter the box within 3 min, it was gently led into the box. The animal remains in the box for an additional 20 seconds before it is removed from the box and gently placed into the home cage. Training is repeated three times a day for four days. The location of the hide box remains the same during every trial but it is shifted between subjects to reduce the potential for unintended intra-maze cues. Phase 2 (Retention): This phase measures retention of spatial memory following a delay. After a two day break from training, each animal is re-tested for a one day, three-trial session using the same hide box location as before. Phase 3 (Reversal): This phase examines memory reversal. On the day following the retention phase, a new hide box location is established 180 degrees to the original location. The same method as before is used and trials were repeated three times a day over two consecutive days.

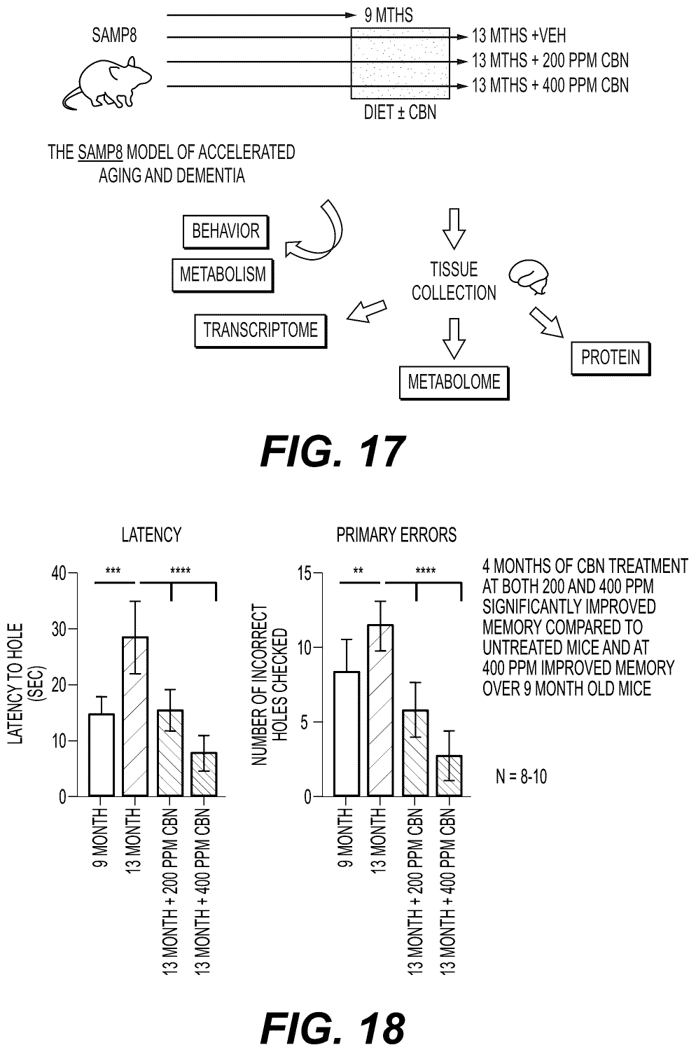

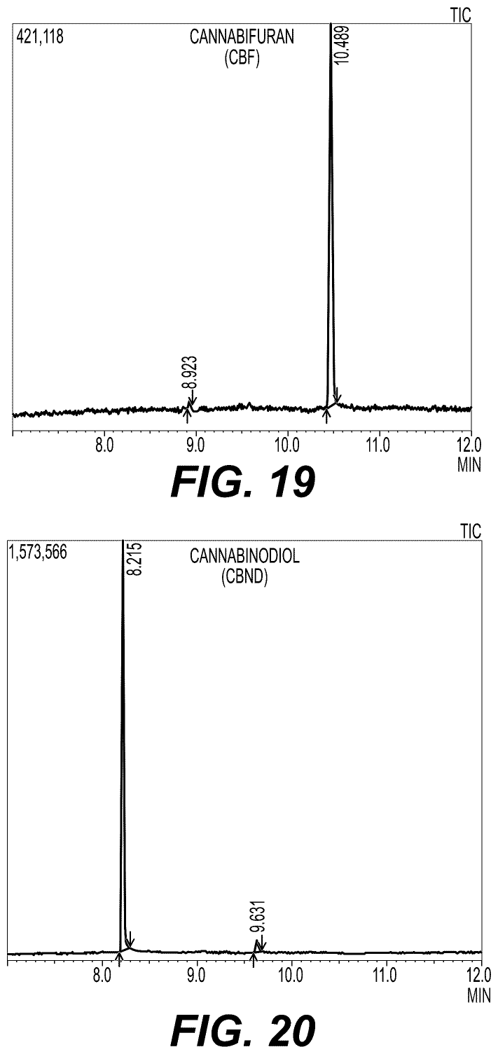

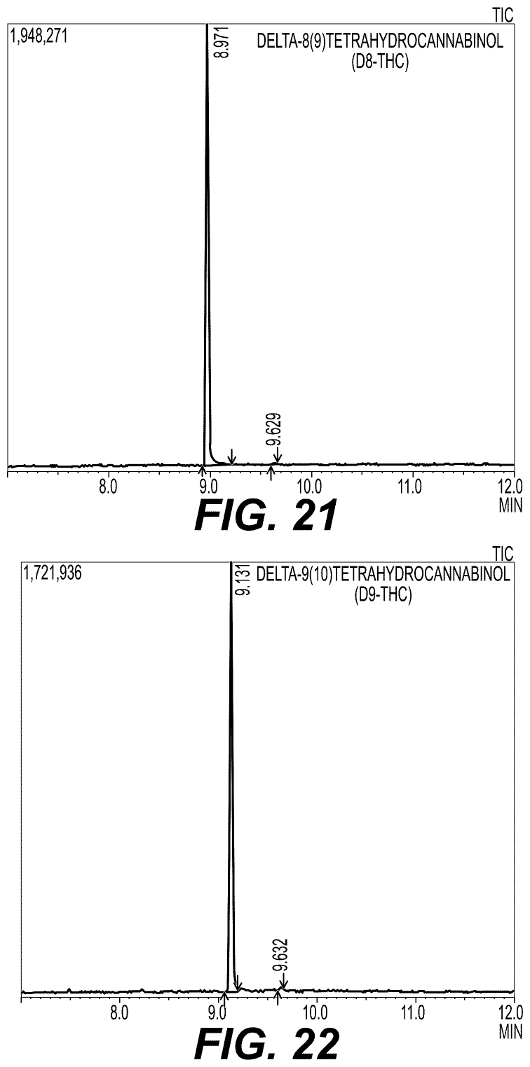

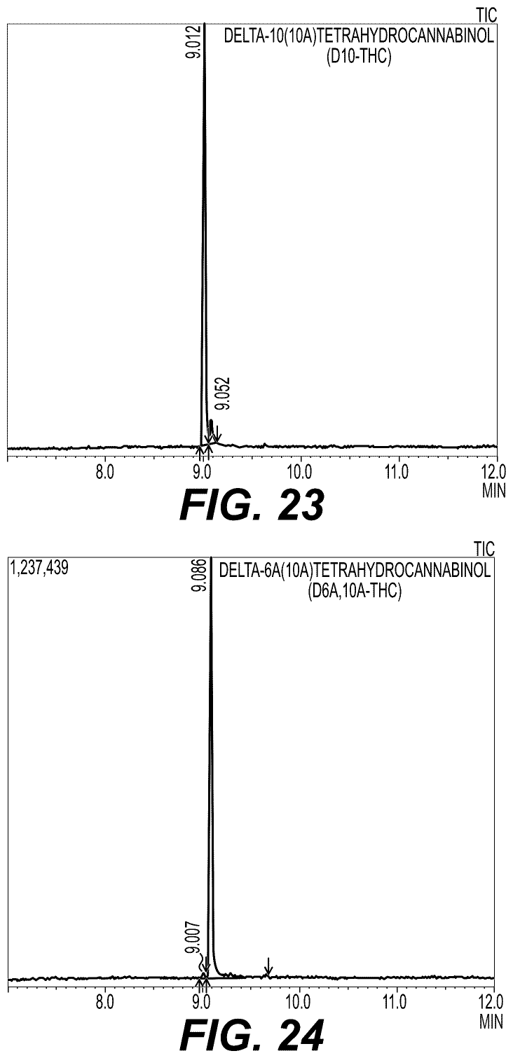

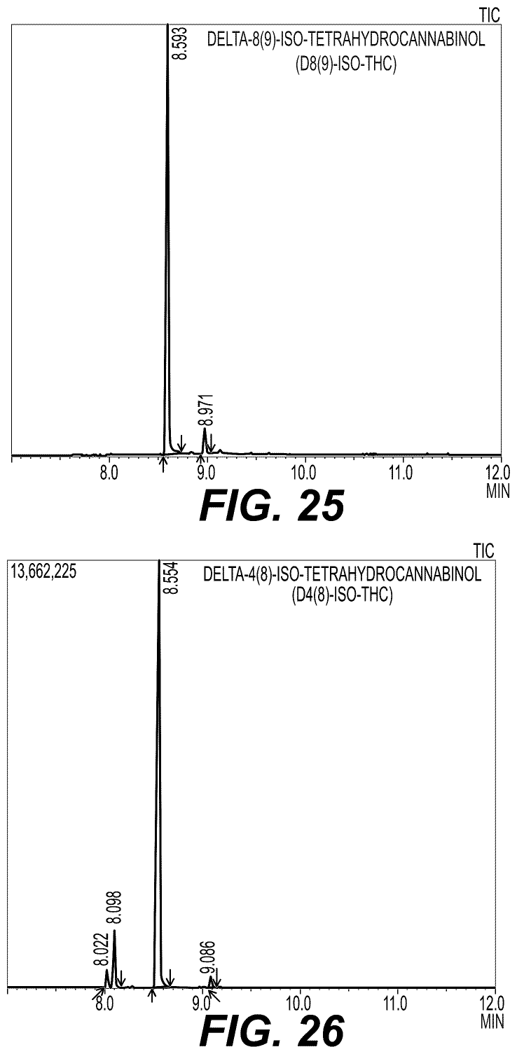

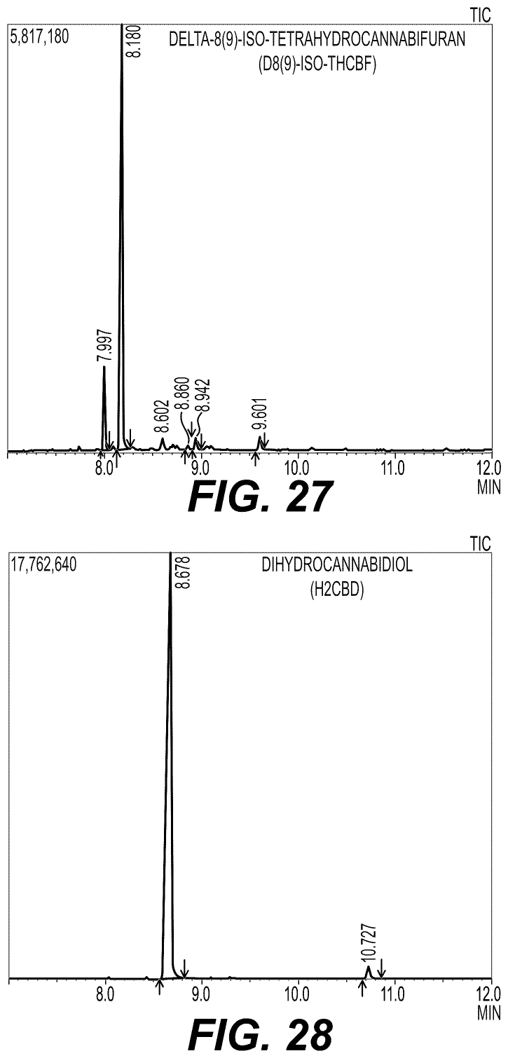

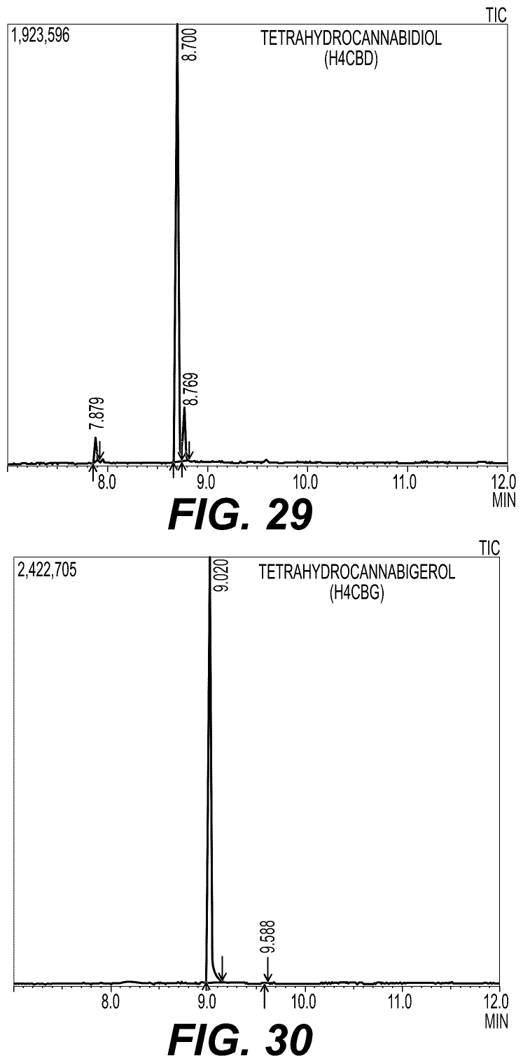

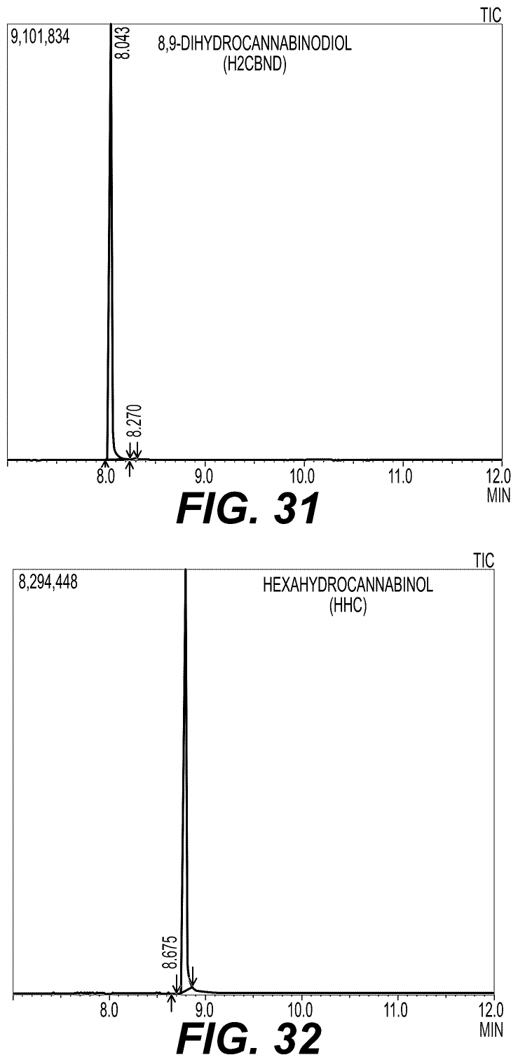

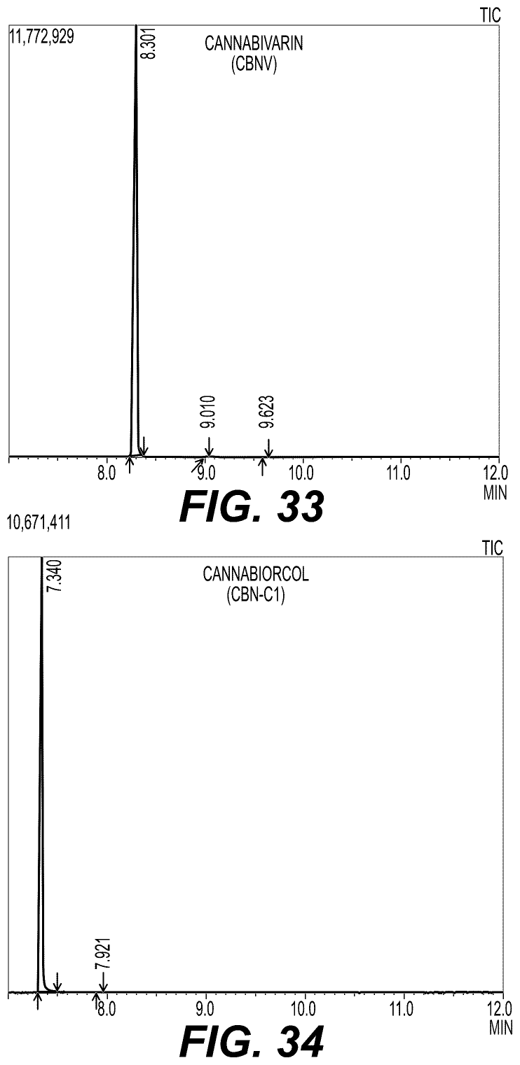

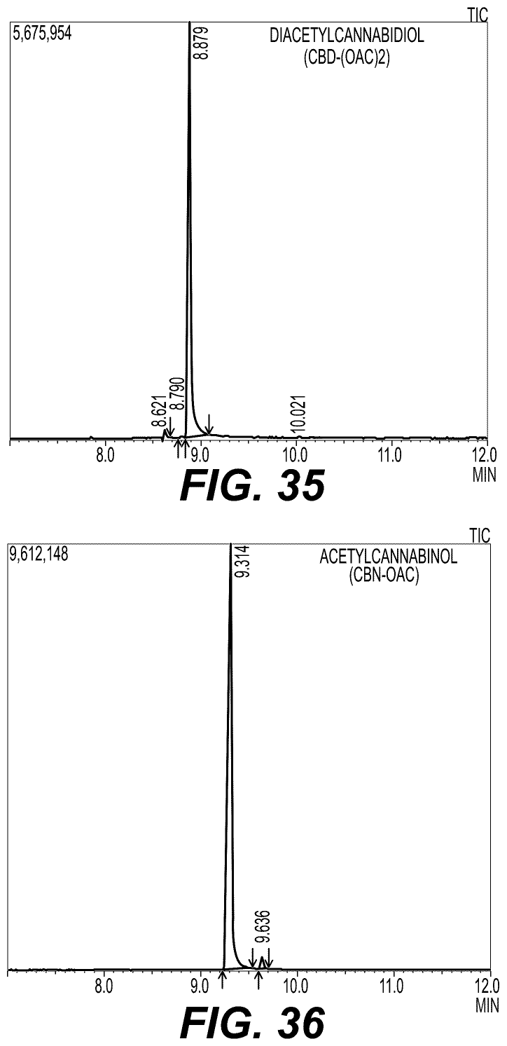

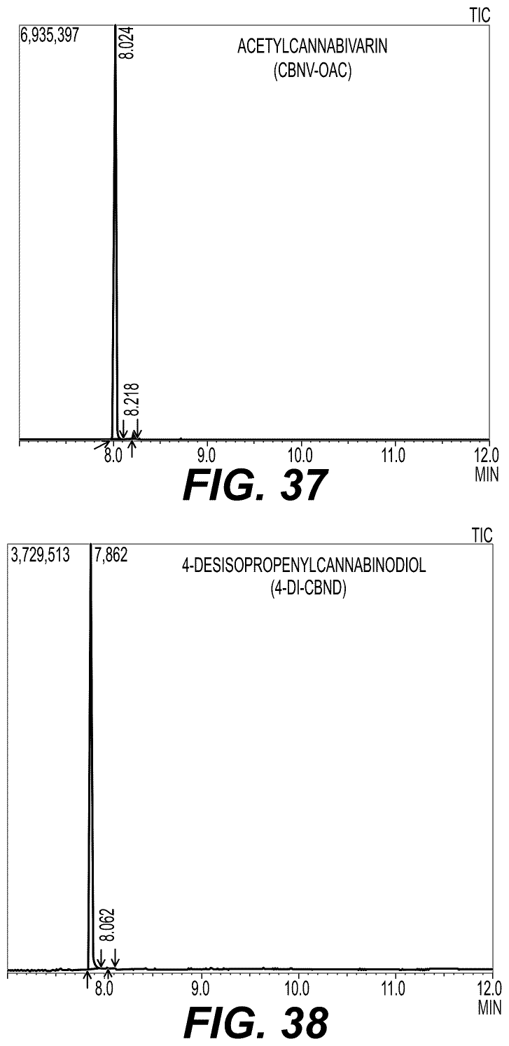

### Study (FIG. 17)

Cannabinol is fed to aged SAMP8 mice and a multiomics approach is used to identify modes of action. Cannabinol is investigated as to whether it reduces metabolic and gene transcription markers of aging in the SAMP8 model of aging and dementia when administered at a late stage of the aging process. It is then investigated whether cannabinol has a mechanism of action that maintains high levels of acetyl-coenzyme A (acetyl-CoA), at least in part, by the inhibition of acetyl-CoA carboxylase 1 (ACC1). Cannabinol is investigated to see whether it increases histone acetylation in cultured neurons and SAMP8 mice at a site on histone H₃ that is required for memory formation.

Testing Aging Associated with Changes in the Hippocampal Transcriptome

To identify age-dependent changes in brain metabolism that are causally associated with dementia, cannabinol is tested in SAMP8 mice. The SAMP8 mice are a model of accelerated aging that develop a progressive, age-associated decline in brain function as well as a number of brain pathologies similar to human dementia and AD patients. Treatment with the diets containing cannabinol start at 9 months of age and continued for four months (13 months of age); the median lifespan of these mice is 15 months. The entire transcriptome of brain hippocampal tissue is studied. Transcriptomic drift analysis, a method to characterize the aging process that measures changes in gene expression at a global level is done. The effects of cannabinol on the expression of individual genes is examined.

Determination of improved cognitive function in SAMP8 mice when administered at advanced stages of dementia. Assessment of spontaneous behavior in the open field assay is accomplished between the 9 and 13 months SAMP8 mice. To investigate the effects of cannabinol on age-associated cognitive decline, mice are tested using the elevated plus maze and the Barnes maze, described above. The elevated plus maze examines disinhibition behavior based on the aversion of normal mice to open spaces. Dementia is clinically associated with disinhibition and AD mouse models tend to exhibit increased disinhibition. The Barnes maze is used to analyze spatial learning and hippocampal-dependent memory. In this assay, mice use visual cues to locate a hidden box (FIG. 18).

### Embodiments

Certain embodiments of the present disclosure are provided as follows.

1. A method of treating a neurodegenerative disease or condition in a subject, comprising administering a therapeutically effective amount of a cannabinoid to the subject.

2. The method of embodiment 1, wherein the cannabinoid is an oxytosis/ferroptosis inhibitor.

3. The method of embodiment 1 or 2, wherein the cannabinoid is a neuroprotector.

4. The method of any one of embodiments 1-3, wherein the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

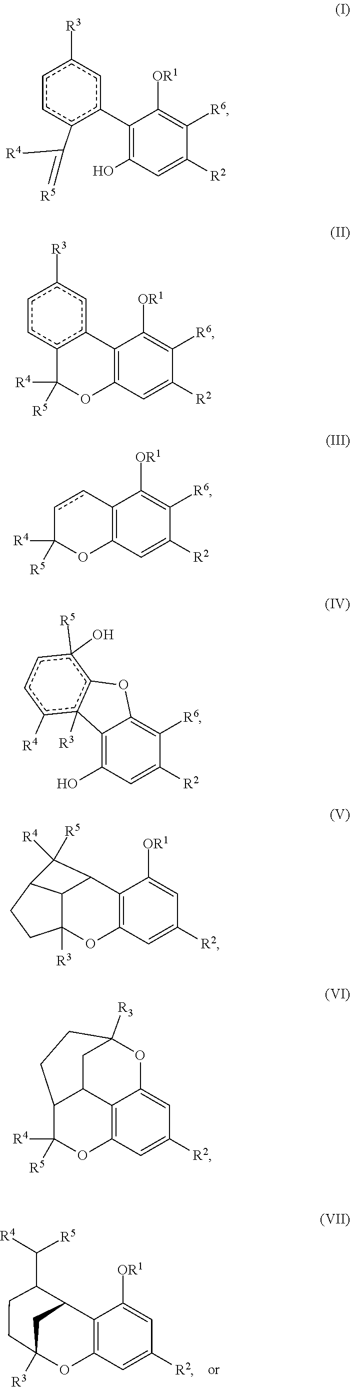

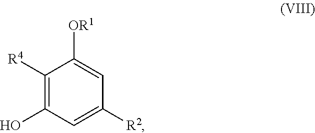

or a pharmaceutically acceptable salt thereof, wherein:
each [symbol] is independently a single bond or a double bond;
R¹ is hydrogen, C₁-C₁₀ alkyl, C(═O)—(C₁-C₁₀ alkyl), or Si(C₁-C₁₀ alkyl)₃;
R² is C₁-C₁₀ alkyl;
R³ is C₁-C₆ alkyl optionally substituted with OH;
R⁴ is C₁-C₁₀ alkyl or C₂-C₁₀ alkenyl;
R⁵ is absent, C₁-C₁₀ alkyl or C₂-C₁₀ alkenyl; and
R⁶ is hydrogen, C₁-C₆ alkyl, C(═O)(OH), or C(═O)—(C₁-C₆ alkyl).

5. The method of embodiment 4, wherein R¹ is hydrogen or C₁-C₃ alkyl.

6. The method of embodiment 4 or 5, wherein R¹ is hydrogen.

7. The method of embodiment 4, wherein R¹ is C(═O)—(C₁-C₃ alkyl).

8. The method of any one of embodiments 4-7, wherein R² is C₁-C₆ alkyl.

9. The method of any one of embodiments 4-8, wherein R² is C₅ alkyl.

10. The method of any one of embodiments 4-9, wherein R³ is C₁-C₃ alkyl optionally substituted with OH.

11. The method of any one of embodiments 4-10, wherein R³ is methyl.

12. The method of any one of embodiments 4-11, wherein R⁴ is C₁-C₆ alkyl.

13. The method of any one of embodiments 4-12, wherein R⁴ is methyl.

14. The method of any one of embodiments 4-13, wherein R⁵ is C₁-C₆ alkyl.

15. The method of any one of embodiments 4-14, wherein R⁵ is methyl.

16. The method of any one of embodiments 4-15, wherein R⁶ is H.

17. The method of any one of embodiments 1-4, wherein the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), iso-hexahydrocannabinol (iso-HHC), 11-hydroxycannabinol (11-OH—CBN), 4-desisopropenyl-cannabinodiol (4-DI-CBND), Abnormal Cannabidiol (Abn-CBD), Abnormal Cannabivarol (Abn-CBNV), Diacetylcannabidiol (CBD-(OAc)₂), Cannabiorcinol (CBN—C₁), Cannabinol ethyl(CBN—C₂), Cannabibutol (CBN—C₄), Cannabihexol (CBN—C₆), Cannabiphorol (CBN—C₇), Cannabivarinol (CBNV or CBN—C₃), Acetylcannabivarol (CBNV—OAc), Δ⁸⁽⁹⁾-iso-tetrahydrocannabifuran (Δ⁸⁽⁹⁾-iso-THCBF), Dihydrocannabielsoin (H₂CBE), Tetrahydrocannabigerol (H₄CBG), and combinations thereof.

18. The method of any one of embodiments 1-4 or 17, wherein the cannabinoid is selected from the group consisting of cannabinol, acetylcannabinol, dihydrocannabinodiol, cannabichromene, cannabigerol, cannabidiol, and combinations thereof.

19. The method of any one of embodiments 1-18, wherein the cannabinoid is selected from the group consisting of cannabinol, acetylcannabinol, dihydrocannabinodiol, and combinations thereof.

20. The method of any one of embodiments 1-19, wherein the cannabinoid has a purity of at least about 95%, at least about 96%, at least about 97%, at least about 98%, at least about 99%, at least about 99.5%, or at least about 99.9%.

21. The method of any one of embodiments 1-20, wherein the cannabinoid is cannabinol.

22. The method of any one of embodiments 1-20, wherein the cannabinoid is cannabifuran.

23. The method of any one of embodiments 1-20, wherein the cannabinoid is tetrahydrocannabidiol.

24. The method of any one of embodiments 1-20, wherein the cannabinoid is tetrahydrocannabigerol.

25. The method of any one of embodiments 1-24, wherein the cannabinoid comprises about 0.5 wt % THC or less.

26. The method of any one of embodiments 1-25, wherein the cannabinoid comprises about 0.3 wt % THC or less.

27. The method of any one of embodiments 1-26, wherein the cannabinoid is substantially free of THC.

28. The method of any one of embodiments 25-27, wherein the THC is Δ⁹⁽¹⁰⁾-THC.

29. The method of any one of embodiments 25-28, wherein the cannabinoid is cannabinol.

30. The method of any one of embodiments 25-28, wherein the cannabinoid is cannabifuran.

31. The method of any one of embodiments 25-28, wherein the cannabinoid is tetrahydrocannabidiol.

32. The method of any one of embodiments 25-28, wherein the cannabinoid is tetrahydrocannabigerol.

33. The method of any one of embodiments 1-32, wherein the subject exhibits one or more risk factors or symptoms associated with a neurodegenerative disease or condition or the development of a neurodegenerative disease or condition.

34. The method of any one of embodiments 1-33, wherein the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis.

35. The method of any one of embodiments 1-34, wherein the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

36. The method of any one of embodiments 1-35, wherein the neurodegenerative disease or condition is an age-associated neurodegenerative disease or condition.

37. The method of any one of embodiments 1-36, wherein the subject is a mammal.

38. The method of any one of embodiments 1-37, wherein the subject is a human.

39. The method of any one of embodiments 1-38, wherein the cannabinoid inhibits oxytosis/ferroptosis independently of cannabinoid receptors.

40. The method of any one of embodiments 1-39, wherein the treating the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics.

41. The method of any one of embodiments 1-40, wherein the cannabinoid is administered in an amount of about 1 mg/kg/day to about 50 mg/kg/day, or about 5 mg/kg/day to about 30 mg/kg/day, or about 10 mg/kg/day to 20 mg/kg/day.

42. A method of treating a disease or disorder associated with mitochondrial dysfunction associated with an aging brain in a subject, comprising administering a therapeutically effective amount of a cannabinoid to the subject.

43. The method of embodiment 42, wherein the cannabinoid is an oxytosis/ferroptosis inhibitor.

44. The method of embodiment 42 or 43, wherein the cannabinoid is a neuroprotector.

45. The method of any one of embodiments 42-44, wherein the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

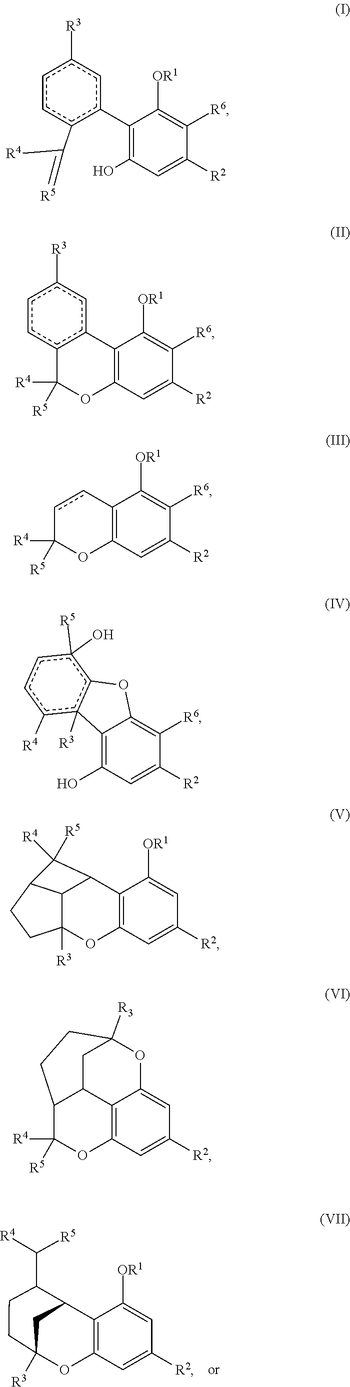

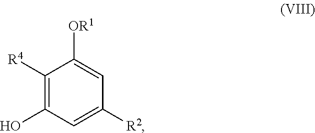

or a pharmaceutically acceptable salt thereof, wherein:
each [symbol] is independently a single bond or a double bond;
R¹ is hydrogen, C₁-C₁₀ alkyl, C(═O)—(C₁-C₁₀ alkyl), or Si(C₁-C₁₀ alkyl)₃;
R² is C₁-C₁₀ alkyl;
R³ is C₁-C₆ alkyl optionally substituted with OH;
R⁴ is C₁-C₁₀ alkyl or C₂-C₁₀ alkenyl;
R⁵ is absent, C₁-C₁₀ alkyl or C₂-C₁₀ alkenyl; and
R⁶ is hydrogen, C₁-C₆ alkyl, C(═O)(OH), or C(═O)—(C₁-C₆ alkyl).

46. The method of any one of embodiments 42-45, wherein the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof.

47. The method of any one of embodiments 42-46, wherein the cannabinoid is selected from the group consisting of cannabinol, acetylcannabinol, dihydrocannabinodiol, cannabichromene, cannabigerol, cannabidiol, and combinations thereof.

48. The method of any one of embodiments 42-47, wherein the cannabinoid is selected from the group consisting of cannabinol, acetylcannabinol, dihydrocannabinodiol, and combinations thereof.

49. The method of any one of embodiments 42-48, wherein the cannabinoid has a purity of at least about 95%, at least about 96%, at least about 97%, at least about 98%, at least about 99%, at least about 99.5%, or at least about 99.9%.

50. The method of any one of embodiments 42-49, wherein the cannabinoid is cannabinol.

51. The method of any one of embodiments 42-49, wherein the cannabinoid is cannabifuran.

52. The method of any one of embodiments 42-49, wherein the cannabinoid is tetrahydrocannabidiol.

53. The method of any one of embodiments 42-49, wherein the cannabinoid is tetrahydrocannabigerol.

54. The method of any one of embodiments 42-53, wherein the cannabinoid comprises about 0.5 wt % THC or less.

55. The method of any one of embodiments 42-54, wherein the cannabinoid comprises about 0.3 wt % THC or less.

56. The method of any one of embodiments 42-55, wherein the cannabinoid is substantially free of THC.

57. The method of any one of embodiments 54-56, wherein the THC is Δ⁹⁽¹⁰⁾-THC.

58. The method of any one of embodiments 54-57, wherein the cannabinoid is cannabinol.

59. The method of any one of embodiments 54-57, wherein the cannabinoid is cannabifuran.

60. The method of any one of embodiments 54-57, wherein the cannabinoid is tetrahydrocannabidiol.

61. The method of any one of embodiments 54-57, wherein the cannabinoid is tetrahydrocannabigerol.

62. The method of any one of embodiments 42-61, wherein the subject exhibits one or more risk factors or symptoms associated with a disease or disorder associated with mitochondrial dysfunction associated with an aging brain or the development of a disease or disorder associated with mitochondrial dysfunction associated with an aging brain.

63. The method of any one of embodiments 42-58, wherein the disease or disorder associated with mitochondrial dysfunction associated with an aging brain is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis.

64. The method of any one of embodiments 42-63, wherein the disease or disorder associated with mitochondrial dysfunction associated with an aging brain is selected from the group consisting of Parkinson's disease, Alzheimer's disease, prion disease, a motor neuron disease (MND), amyotrophic lateral sclerosis (ALS), and Huntington's disease (HD).

65. The method of any one of embodiments 42-64, wherein the subject is a mammal.

66. The method of any one of embodiments 42-65, wherein the subject is a human.

67. The method of any one of embodiments 42-66, wherein the cannabinoid inhibits oxytosis/ferroptosis independently of cannabinoid receptors.

68. The method of any one of embodiments 42-67, wherein the treating the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal β-amyloid in neuronal cells, and regulation of mitochondrial dynamics.

69. The method of any one of embodiments 42-68, wherein the cannabinoid is administered in an amount of about 1 mg/kg/day to about 50 mg/kg/day, or about 5 mg/kg/day to about 30 mg/kg/day, or about 10 mg/kg/day to 20 mg/kg/day.

70. A method of inhibiting oxytosis/ferroptosis in a subject, comprising administering a cannabinoid to the subject.

71. The method of embodiment 70, wherein the cannabinoid is a neuroprotector.

72. The method of embodiment 70 or 71, wherein the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

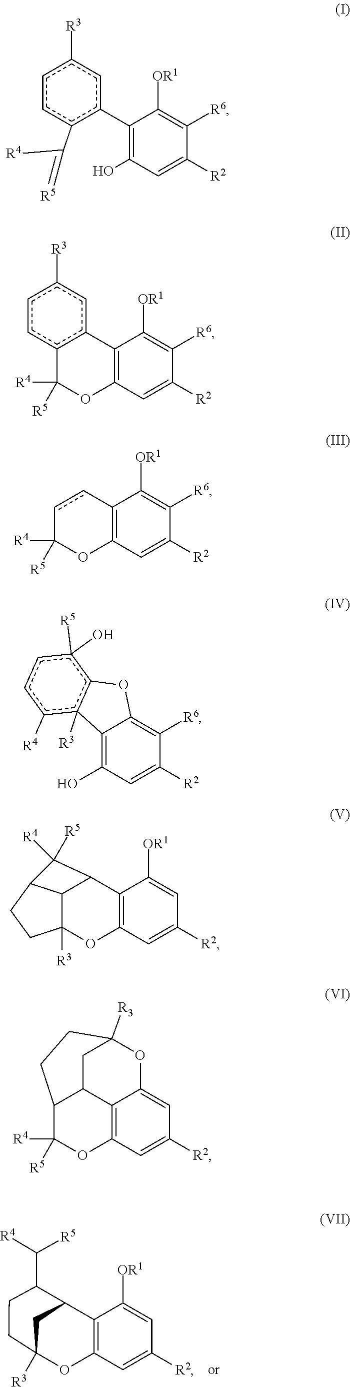

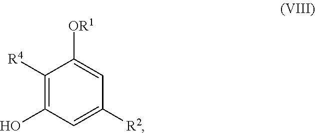

or a pharmaceutically acceptable salt thereof, wherein:
each [symbol] is independently a single bond or a double bond;
R¹ is hydrogen, C₁-C₁₀ alkyl, C(═O)—(C₁-C₁₀ alkyl), or Si(C₁-C₁₀ alkyl)₃;
R² is C₁-C₁₀ alkyl;
R³ is C₁-C₆ alkyl optionally substituted with OH;
R⁴ is C₁-C₁₀ alkyl or C₂-C₁₀ alkenyl;
R⁵ is absent, C₁-C₁₀ alkyl or C₂-C₁₀ alkenyl; and
R⁶ is hydrogen, C₁-C₆ alkyl, C(═O)(OH), or C(═O)—(C₁-C₆ alkyl).

73. The method of any one of embodiments 70-72, wherein the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof.

74. The method of any one of embodiments 70-73, wherein the cannabinoid is selected from the group consisting of cannabinol, acetylcannabinol, dihydrocannabinodiol, cannabichromene, cannabigerol, cannabidiol, and combinations thereof.

75. The method of any one of embodiments 70-74, wherein the cannabinoid is selected from the group consisting of cannabinol, acetylcannabinol, dihydrocannabinodiol, and combinations thereof.

76. The method of any one of embodiments 70-75, wherein the cannabinoid has a purity of at least about 95%, at least about 96%, at least about 97%, at least about 98%, at least about 99%, at least about 99.5%, or at least about 99.9%.

77. The method of any one of embodiments 70-76, wherein the cannabinoid is cannabinol.

78. The method of any one of embodiments 70-76, wherein the cannabinoid is cannabifuran.

79. The method of any one of embodiments 70-76, wherein the cannabinoid is tetrahydrocannabidiol.

80. The method of any one of embodiments 70-76, wherein the cannabinoid is tetrahydrocannabigerol.

81. The method of any one of embodiments 70-80, wherein the cannabinoid comprises about 0.5 wt % THC or less.

82. The method of any one of embodiments 70-81, wherein the cannabinoid comprises about 0.3 wt % THC or less.

83. The method of any one of embodiments 70-82, wherein the cannabinoid is substantially free of THC.

84. The method of any one of embodiments 81-83, wherein the THC is Δ⁹⁽¹⁰⁾-THC.

85. The method of any one of embodiments 81-84, wherein the cannabinoid is cannabinol.

86. The method of any one of embodiments 81-84, wherein the cannabinoid is cannabifuran.

87. The method of any one of embodiments 81-84, wherein the cannabinoid is tetrahydrocannabidiol.

88. The method of any one of embodiments 81-84, wherein the cannabinoid is tetrahydrocannabigerol.

89. The method of any one of embodiments 70-88, wherein the subject has a neurodegenerative disease or condition.

90. The method of any one of embodiments 70-89, wherein the subject exhibits one or more risk factors or symptoms associated with a neurodegenerative disease or condition or the development of a neurodegenerative disease or condition.

91. The method of embodiment 89 or 90, wherein the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis.

92. The method of any one of embodiments 89-91, wherein the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

93. The method of any one of embodiments 70-92, wherein the subject is a mammal.

94. The method of any one of embodiments 70-93, wherein the subject is a human.

95. The method of any one of embodiments 70-94, wherein the cannabinoid inhibits oxytosis/ferroptosis independently of cannabinoid receptors.

96. The method of any one of embodiments 70-95, wherein inhibiting oxytosis/ferroptosis in the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal ß-amyloid in neuronal cells, and regulation of mitochondrial dynamics.

97. The method of any one of embodiments 70-96, wherein the cannabinoid is administered in an amount of about 1 mg/kg/day to about 50 mg/kg/day, or about 5 mg/kg/day to about 30 mg/kg/day, or about 10 mg/kg/day to 20 mg/kg/day.

98. A method of protecting nerve cells from oxytosis/ferroptosis in a subject, comprising administering a cannabinoid to the subject.

99. The method of embodiment 98, wherein the cannabinoid is an oxytosis/ferroptosis inhibitor.

100. The method of embodiment 98 or 99, wherein the cannabinoid is a neuroprotector.

101. The method of any one of embodiments 98-100, wherein the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII):

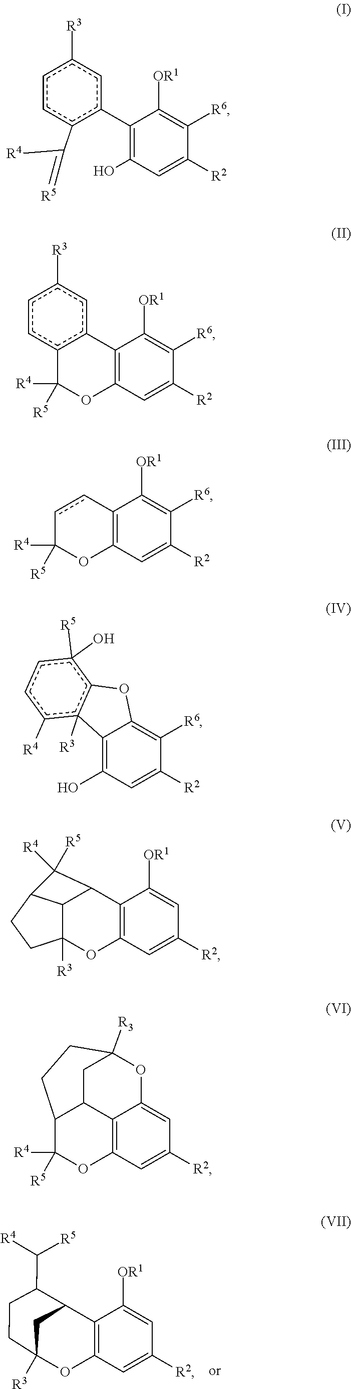

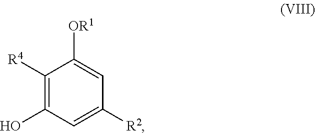

or a pharmaceutically acceptable salt thereof, wherein:
each [symbol] is independently a single bond or a double bond;
R¹ is hydrogen, C₁-C₁₀ alkyl, C(═O)—(C₁-C₁₀ alkyl), or Si(C₁-C₁₀ alkyl)₃;
R² is C₁-C₁₀ alkyl;
R³ is C₁-C₆ alkyl optionally substituted with OH;
R⁴ is C₁-C₁₀ alkyl or C₂-C₁₀ alkenyl;
R⁵ is absent, C₁-C₁₀ alkyl or C₂-C₁₀ alkenyl; and
R⁶ is hydrogen, C₁-C₆ alkyl, C(═O)(OH), or C(═O)—(C₁-C₆ alkyl).

102. The method of any one of embodiments 98-101, wherein the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof.

103. The method of any one of embodiments 98-102, wherein the cannabinoid is selected from the group consisting of cannabinol, acetylcannabinol, dihydrocannabinodiol, cannabichromene, cannabigerol, cannabidiol, and combinations thereof.

104. The method of any one of embodiments 98-103, wherein the cannabinoid is selected from the group consisting of cannabinol, acetylcannabinol, dihydrocannabinodiol, and combinations thereof.

105. The method of any one of embodiments 98-104, wherein the cannabinoid has a purity of at least about 95%, at least about 96%, at least about 97%, at least about 98%, at least about 99%, at least about 99.5%, or at least about 99.9%.

106. The method of any one of embodiments 98-105, wherein the cannabinoid is cannabinol.

107. The method of any one of embodiments 98-105, wherein the cannabinoid is cannabifuran.

108. The method of any one of embodiments 98-105, wherein the cannabinoid is tetrahydrocannabidiol.

109. The method of any one of embodiments 98-105, wherein the cannabinoid is tetrahydrocannabigerol.

110. The method of any one of embodiments 98-109, wherein the cannabinoid comprises about 0.5 wt % THC or less.

111. The method of any one of embodiments 98-110, wherein the cannabinoid comprises about 0.3 wt % THC or less.

112. The method of any one of embodiments 98-111, wherein the cannabinoid is substantially free of THC.

113. The method of any one of embodiments 98-112, wherein the THC is Δ⁹⁽¹⁰⁾-THC.

114. The method of any one of embodiments 110-113, wherein the cannabinoid is cannabinol.

115. The method of any one of embodiments 110-113, wherein the cannabinoid is cannabifuran.

116. The method of any one of embodiments 110-113, wherein the cannabinoid is tetrahydrocannabidiol.

116. The method of any one of embodiments 110-113, wherein the cannabinoid is tetrahydrocannabigerol.

117. The method of any one of embodiments 98-116, wherein administration of the cannabinoid preserves mitochondrial function.

118. The method of any one of embodiments 98-117, wherein the subject has a neurodegenerative disease or condition.

119. The method of any one of embodiments 98-118, wherein the subject exhibits one or more risk factors or symptoms associated with a neurodegenerative disease or condition or the development of a neurodegenerative disease or condition.

120. The method of embodiment 118 or 119, wherein the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis.

121. The method of any one of embodiments 118-120, wherein the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

122. The method of any one of embodiments 98-121, wherein the subject is a mammal.

123. The method of any one of embodiments 98-122, wherein the subject is a human.

124. The method of any one of embodiments 98-123, wherein the cannabinoid inhibits oxytosis/ferroptosis independently of cannabinoid receptors.

125. The method of any one of embodiments 98-124, wherein inhibiting oxytosis/ferroptosis in the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal ß-amyloid in neuronal cells, and regulation of mitochondrial dynamics.

126. The method of any one of embodiments 98-125, wherein the cannabinoid is administered in an amount of about 1 mg/kg/day to about 50 mg/kg/day, or about 5 mg/kg/day to about 30 mg/kg/day, or about 10 mg/kg/day to 20 mg/kg/day.

127. A method of treating a neurodegenerative disease or condition in a subject, comprising administering a therapeutically effective amount of a cannabinoid to the subject, wherein the cannabinoid inhibits oxytosis/ferroptosis.

128. The method of embodiment 127, wherein the cannabinoid inhibits oxytosis/ferroptosis independently of cannabinoid receptors.

129. The method of embodiment 127 or 128, wherein the cannabinoid has a purity of at least about 95%, at least about 96%, at least about 97%, at least about 98%, at least about 99%, at least about 99.5%, or at least about 99.9%.

130. The method of any one of embodiments 127-129, wherein the cannabinoid is cannabinol.

131. The method of any one of embodiments 127-129, wherein the cannabinoid is cannabifuran.

132. The method of any one of embodiments 127-129, wherein the cannabinoid is tetrahydrocannabidiol.

133. The method of any one of embodiments 127-129, wherein the cannabinoid is tetrahydrocannabigerol.

134. The method of any one of embodiments 127-129, wherein the cannabinoid comprises about 0.5 wt % THC or less.

135. The method of any one of embodiments 127-129, wherein the cannabinoid comprises about 0.3 wt % THC or less.

136. The method of any one of embodiments 127-129, wherein the cannabinoid is substantially free of THC.

137. The method of any one of embodiments 127-129, wherein the THC is Δ⁹⁽¹⁰⁾-THC.

138. The method of any one of embodiments 134-137, wherein the cannabinoid is cannabinol.

139. The method of any one of embodiments 134-137, wherein the cannabinoid is cannabifuran.

140. The method of any one of embodiments 134-137, wherein the cannabinoid is tetrahydrocannabidiol.

141. The method of any one of embodiments 134-137, wherein the cannabinoid is tetrahydrocannabigerol.

142. The method of any one of embodiments 127-141, wherein the subject exhibits one or more risk factors or symptoms associated with a neurodegenerative disease or condition or the development of a neurodegenerative disease or condition.

143. The method of any one of embodiments 127-142, wherein the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy,

Kennedy's disease, Alexander's disease, Cockayne syndrome, and Icelandic hereditary cerebral hemorrhage with amyloidosis.

144. The method of any one of embodiments 127-143, wherein the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

145. The method of any one of embodiments 127-144, wherein the neurodegenerative disease or condition is an age-associated neurodegenerative disease or condition.

146. A method of treating a disease or disorder associated with mitochondrial dysfunction associated with an aging brain in a subject, comprising administering a therapeutically effective amount of a cannabinoid to the subject.

147. The method of embodiment 146, wherein the cannabinoid has a purity of at least about 95%, at least about 96%, at least about 97%, at least about 98%, at least about 99%, at least about 99.5%, or at least about 99.9%.

148. The method of embodiment 146 or 147, wherein the cannabinoid is cannabinol.

149. The method of embodiment 146 or 147, wherein the cannabinoid is cannabifuran.

150. The method of embodiment 146 or 147, wherein the cannabinoid is tetrahydrocannabidiol.

151. The method of embodiment 146 or 147, wherein the cannabinoid is tetrahydrocannabigerol.

152. The method of any one of embodiments 146-151, wherein the cannabinoid comprises about 0.5 wt % THC or less.

153. The method of any one of embodiments 146-152, wherein the cannabinoid comprises about 0.3 wt % THC or less.

154. The method of any one of embodiments 146-153, wherein the cannabinoid is substantially free of THC.

155. The method of any one of embodiments 146-154, wherein the THC is Δ⁹⁽¹⁰⁾-THC.

156. The method of any one of embodiments 152-155, wherein the cannabinoid is cannabinol.

157. The method of any one of embodiments 152-155, wherein the cannabinoid is cannabifuran.

158. The method of any one of embodiments 152-155, wherein the cannabinoid is tetrahydrocannabidiol.

159. The method of any one of embodiments 152-155, wherein the cannabinoid is tetrahydrocannabigerol.

160. The method of any one of embodiments 146-159, wherein the subject exhibits one or more risk factors or symptoms associated with a disease or disorder associated with mitochondrial dysfunction associated with an aging brain or the development of a disease or disorder associated with mitochondrial dysfunction associated with an aging brain.

161. The method of any one of embodiments 146-160, wherein the disease or disorder associated with mitochondrial dysfunction associated with an aging brain is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis.

162. The method of any one of embodiments 146-161, wherein the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

163. The method of embodiment 148 or 156, wherein the cannabinol is administered in an amount of about 1 mg/kg/day to about 50 mg/kg/day, or about 5 mg/kg/day to about 30 mg/kg/day, or about 10 mg/kg/day to 20 mg/kg/day.

164. The method of embodiment 149 or 157, wherein the cannabifuran is administered in an amount of about 1 mg/kg/day to about 50 mg/kg/day, or about 5 mg/kg/day to about 30 mg/kg/day, or about 10 mg/kg/day to 20 mg/kg/day.

165. The method of embodiment 150 or 158, wherein the tetrahydrocannabidiol is administered in an amount of about 1 mg/kg/day to about 50 mg/kg/day, or about 5 mg/kg/day to about 30 mg/kg/day, or about 10 mg/kg/day to 20 mg/kg/day.

166. The method of embodiment 151 or 159, wherein the tetrahydrocannabigerol is administered in an amount of about 1 mg/kg/day to about 50 mg/kg/day, or about 5 mg/kg/day to about 30 mg/kg/day, or about 10 mg/kg/day to 20 mg/kg/day.

167. A method of protecting nerve cells from oxytosis/ferroptosis in a subject, comprising administering a cannabinoid to the subject.

168. The method of embodiment 167, wherein the cannabinoid has a purity of at least about 95%, at least about 96%, at least about 97%, at least about 98%, at least about 99%, at least about 99.5%, or at least about 99.9%.

169. The method of embodiment 167 or 168, wherein the cannabinoid is cannabinol.

170. The method of embodiment 167 or 168, wherein the cannabinoid is cannabifuran.

171. The method of embodiment 167 or 168, wherein the cannabinoid is tetrahydrocannabidiol.

172. The method of embodiment 167 or 168, wherein the cannabinoid is tetrahydrocannabigerol.

173. The method of any one of embodiments 167-172, wherein the cannabinoid comprises about 0.5 wt % THC or less.

174. The method of any one of embodiments 167-173, wherein the cannabinoid comprises about 0.3 wt % THC or less.

175. The method of any one of embodiments 167-174, wherein the cannabinoid is substantially free of THC.

176. The method of any one of embodiments 173-175, wherein the THC is Δ⁹⁽¹⁰⁾-THC.

177. The method of any one of embodiments 173-175, wherein the cannabinoid is cannabinol.

178. The method of any one of embodiments 173-175, wherein the cannabinoid is cannabifuran.

179. The method of any one of embodiments 173-175, wherein the cannabinoid is tetrahydrocannabidiol.

180. The method of any one of embodiments 173-175, wherein the cannabinoid is tetrahydrocannabigerol.

181. The method of any one of embodiments 167-180, wherein the subject exhibits one or more risk factors or symptoms associated with a neurodegenerative disease or condition or the development of a neurodegenerative disease or condition.

182. The method of embodiment 181, wherein the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis.

183. The method of embodiment 181 or 182, wherein the neurodegenerative disease or condition is selected from the group consisting of Parkinson's disease, Alzheimer's disease, prion disease, a motor neuron disease (MND), amyotrophic lateral sclerosis (ALS), and Huntington's disease (HD).

184. The method of any one of embodiments 181-183, wherein the neurodegenerative disease or condition is an age-associated neurodegenerative disease or condition.

185. The method of embodiments 169 or 177, wherein the cannabinol is administered in an amount of about 1 mg/kg/day to about 50 mg/kg/day, or about 5 mg/kg/day to about 30 mg/kg/day, or about 10 mg/kg/day to 20 mg/kg/day.

186. The method of embodiments 170 or 178, wherein the cannabifuran is administered in an amount of about 1 mg/kg/day to about 50 mg/kg/day, or about 5 mg/kg/day to about 30 mg/kg/day, or about 10 mg/kg/day to 20 mg/kg/day.

187. The method of embodiments 171 or 179, wherein the tetrahydrocannabidiol is administered in an amount of about 1 mg/kg/day to about 50 mg/kg/day, or about 5 mg/kg/day to about 30 mg/kg/day, or about 10 mg/kg/day to 20 mg/kg/day.

188. The method of embodiments 172 or 180, wherein the tetrahydrocannabigerol is administered in an amount of about 1 mg/kg/day to about 50 mg/kg/day, or about 5 mg/kg/day to about 30 mg/kg/day, or about 10 mg/kg/day to 20 mg/kg/day.

1. A method of treating a neurodegenerative disease or condition in a subject, comprising administering a therapeutically effective amount of a cannabinoid to the subject.

2. The method of claim 1, wherein the cannabinoid is an oxytosis/ferroptosis inhibitor.

3. The method of claim 1, wherein the cannabinoid is a neuroprotector.

4. The method of claim 1, wherein the cannabinoid is a compound of Formula (I), a compound of Formula (II), a compound of Formula (III), a compound of Formula (IV), a compound of Formula (V), a compound of Formula (VI), a compound of Formula (VII), or a compound of Formula (VIII): 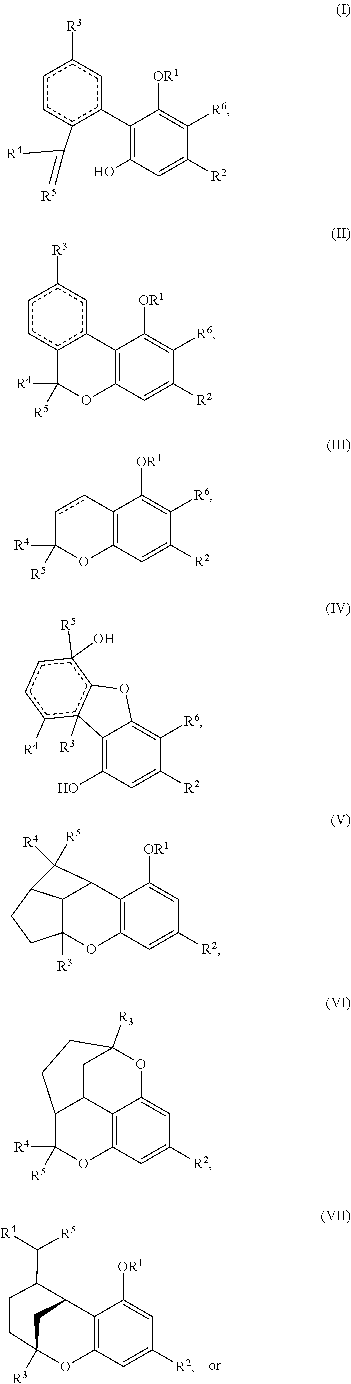 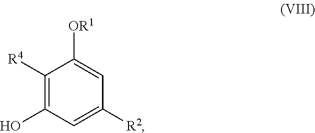
or a pharmaceutically acceptable salt thereof, wherein:
each [symbol] is independently a single bond or a double bond;
R¹ is hydrogen, C₁-C₁₀ alkyl, C(═O)—(C₁-C₁₀ alkyl), or Si(C₁-C₁₀ alkyl)₃;
R² is C₁-C₁₀ alkyl;
R³ is C₁-C₆ alkyl optionally substituted with OH;
R⁴ is C₁-C₁₀ alkyl or C₂-C₁₀ alkenyl;
R⁵ is absent, C₁-C₁₀ alkyl or C₂-C₁₀ alkenyl; and
R⁶ is hydrogen, C₁-C₆ alkyl, C(═O)(OH), or C(═O)—(C₁-C₆ alkyl).

5. The method of claim 4, wherein R¹ is hydrogen or C₁-C₃ alkyl.

6. (canceled)

7. The method of claim 4, wherein R¹ is C(═O)—(C₁-C₃ alkyl).

8. The method of claim 4, wherein R² is C₁-C₆ alkyl.

9. The method of claim 4, wherein R² is C₅ alkyl.

10. The method of claim 4, wherein R³ is C₁-C₃ alkyl optionally substituted with OH.

11. The method of claim 4, wherein R³ is methyl or CH₂OH.

12. The method of claim 4, wherein R⁴ is C₁-C₆ alkyl.

13. The method of claim 4, wherein R⁴ is methyl.

14. The method of claim 4, wherein R⁵ is C₁-C₆ alkyl.

15. The method of claim 4, wherein R⁵ is methyl.

16. The method of claim 4, wherein R⁶ is H.

17. The method of claim 1, wherein the cannabinoid is selected from the group consisting of cannabichromene (CBC), cannabichromenic acid (CBCA), cannabichromenevarin (CBCV), cannabidiol (CBD), cannabidiolic acid (CBDA), cannabidivarol (CBDV), cannabielsoin (CBE), cannabifuran (CBF), cannabigerol (CBG), cannabigerolic acid (CBGA), cannabigerovarinol (CBGV), cannabicyclol (CBL), cannabinol (CBN), cannabinolic acid (CBNA), cannabivarol (CBNV), acetylcannabinol (CBN—OAc), cannabinodiol (CBND), methoxycannabinol (CBN—OMe), cannabicitran (CBT), dehydrocannabifuran (DHCBF), dihydrocannabinodiol (H₂CBND), dihydrocannabidiol (H₂CBD), tetrahydrocannabidiol (H₄CBD), Δ⁸⁽⁹⁾-iso-tetrahydrocannabinol (Δ⁸⁽⁹⁾-iso-THC), Δ⁴⁽⁸⁾-iso-tetrahydrocannabinol (Δ⁴⁽⁸⁾-iso-THC), Δ⁴⁽⁵⁾-iso-tetrahydrocannabinol (Δ⁴⁽⁵⁾-iso-THC), hexahydrocannabinol (HHC), Δ⁸⁽⁹⁾-tetrahydrocannabinol (Δ⁸⁽⁹⁾-THC), Δ⁹⁽¹⁰⁾-tetrahydrocannabinol (Δ⁹⁽¹⁰⁾-THC), Δ⁹⁽¹⁰⁾-tetrahydrocannabinolic acid (Δ⁹⁽¹⁰⁾-THCA), Δ⁹⁽¹⁰⁾-tetrahydrocannabinovarol (Δ⁹⁽¹⁰⁾-THCV), Δ^{10(10a)}-tetrahydrocannabinol (Δ^{10(10a)}-THC), Δ^{6a(10a)}-tetrahydrocannabinol (Δ^{6a(10a)}-THC), iso-hexahydrocannabinol (iso-HHC), 11-hydroxycannabinol (11-OH—CBN), 4-desisopropenyl-cannabinodiol (4-DI-CBND), abnormal cannabidiol (Abn-CBD), abnormal cannabivarol (Abn-CBNV), diacetylcannabidiol (CBD-(OAc)₂), cannabiorcinol (CBN—C₁), cannabinol ethyl(CBN—C₂), cannabibutol (CBN—C₄), cannabihexol (CBN—C₆), cannabiphorol (CBN—C₇), cannabivarinol (CBNV or CBN—C₃), acetylcannabivarol (CBNV—OAc), Δ⁸⁽⁹⁾-iso-tetrahydrocannabifuran (Δ⁸⁽⁹⁾-iso-THCBF), dihydrocannabielsoin (H₂CBE), tetrahydrocannabigerol (H₄CBG), and combinations thereof.

18-19. (canceled)

20. The method of claim 1, wherein the cannabinoid has a purity of at least about 95%, at least about 96%, at least about 97%, at least about 98%, at least about 99%, at least about 99.5%, or at least about 99.9%.

21. The method of claim 1, wherein the cannabinoid is cannabinol.

22. The method of claim 1, wherein the cannabinoid is cannabifuran.

23. The method of claim 1, wherein the cannabinoid is tetrahydrocannabidiol.

24. The method of claim 1, wherein the cannabinoid is tetrahydrocannabigerol.

25. The method of claim 1, wherein the cannabinoid comprises about 0.5 wt % THC or less.

26-32. (canceled)

33. The method of claim 1, wherein the subject exhibits one or more risk factors or symptoms associated with a neurodegenerative disease or condition or the development of a neurodegenerative disease or condition.

34. The method of claim 1, wherein the neurodegenerative disease or condition is selected from the group consisting of Parkinson's disease, Alzheimer's disease, prion disease, a motor neuron disease (MND), amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE), a synucleinopathy, a tauopathy, a spongiform encephalopathy, familial amyloidotic polyneuropathy, Dutch hereditary cerebral hemorrhage with amyloidosis, congophilic angiopathy, corticobasal degeneration, Pick's disease, progressive supranuclear palsy, Creutzfeldt-Jacob disease, Gerstmann-Sträussler-Schneiker syndrome, fatal familial insomnia, kuru, bovine spongiform encephalopathy, scrapie, chronic wasting disease, Lewy body variant of Alzheimer's disease, diffuse Lewy body disease, dementia with Lewy bodies, multiple system atrophy, neurodegeneration with brain iron accumulation type L diffuse Lewy body disease, frontotemporal lobar degeneration, hereditary dentatorubral-pallidoluysian atrophy, Kennedy's disease, Alexander's disease, Cockayne syndrome, and Icelandic hereditary cerebral hemorrhage with amyloidosis.

35. (canceled)

36. The method of claim 1, wherein the neurodegenerative disease or condition is an age-associated neurodegenerative disease or condition.

37-38. (canceled)

39. The method of claim 2, wherein the cannabinoid inhibits oxytosis/ferroptosis independently of cannabinoid receptors.

40. The method of claim 1, wherein the treating the subject results in one or more of suppression of mitochondrial oxidative stress in oxytosis/ferroptosis, maintenance of mitochondrial calcium homeostasis in oxytosis/ferroptosis, modulation of oxidative phosphorylation system, restoration of mitochondrial bioenergetics, promotion of mitochondrial biogenesis, reduction of intraneuronal ß-amyloid in neuronal cells, and regulation of mitochondrial dynamics.

41. The method of claim 1, wherein the cannabinoid is administered in an amount of about 1 mg/kg/day to about 50 mg/kg/day, or about 5 mg/kg/day to about 30 mg/kg/day, or about 10 mg/kg/day to 20 mg/kg/day.

42. A method of treating a disease or disorder associated with mitochondrial dysfunction associated with an aging brain in a subject, comprising administering a therapeutically effective amount of a cannabinoid to the subject.

43-69. (canceled)

70. A method of inhibiting oxytosis/ferroptosis in a subject, comprising administering a cannabinoid to the subject.

71-97. (canceled)

98. A method of protecting nerve cells from oxytosis/ferroptosis in a subject, comprising administering a cannabinoid to the subject.

99-126. (canceled)

127. A method of treating a neurodegenerative disease or condition in a subject, comprising administering a therapeutically effective amount of a cannabinoid to the subject, wherein the cannabinoid inhibits oxytosis/ferroptosis.

128-145. (canceled)

146. A method of treating a disease or disorder associated with mitochondrial dysfunction associated with an aging brain in a subject, comprising administering a therapeutically effective amount of a cannabinoid to the subject.

147-166. (canceled)

167. A method of protecting nerve cells from oxytosis/ferroptosis in a subject, comprising administering a cannabinoid to the subject.

168-188. (canceled)
